SEQUENCE LISTING (21 sequences)

SEQ ID NO: 1 - protein, 585 aa, Homo sapiens [sequence omitted]

SEQ ID NO: 2 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 3 - protein, 19 aa, Homo sapiens
MFSMRIVCLVLSVVGTAWT

SEQ ID NO: 4 - protein, 16 aa, synthetic construct
  REGION 1..16: note = Synthetic
GGGGSGGGGSGGGGLC

SEQ ID NO: 5 - protein, 16 aa, synthetic construct
  REGION 1..16: note = Synthetic
GGGGSGGGGSGGGLGC

SEQ ID NO: 6 - protein, 26 aa, synthetic construct
  REGION 1..26: note = Synthetic
AAASAEAAAKEAAAKEAAAKAAAGSG

SEQ ID NO: 7 - protein, 15 aa, synthetic construct
  REGION 1..15: note = Synthetic
GGGGSGGGGSGGGLG

SEQ ID NO: 8 - protein, 16 aa, synthetic construct
  REGION 1..16: note = Synthetic
GGGGSGGGGSGGGGSC

SEQ ID NO: 9 - protein, 6 aa, synthetic construct
  REGION 1..6: note = Synthetic
GGGGSC

SEQ ID NO: 10 - protein, 28 aa, synthetic construct
  REGION 1..28: note = Synthetic
AAASAEAAAKEAAAKEAAAKAAAGSGLC

SEQ ID NO: 11 - protein, 6 aa, synthetic construct
  REGION 1..6: note = Synthetic
GGGLGC

SEQ ID NO: 12 - protein, 15 aa, synthetic construct
  REGION 1..15: note = Synthetic
GGGGSGGGGSGGGGS

SEQ ID NO: 13 - protein, 5 aa, synthetic construct
  REGION 1..5: note = Synthetic
GGGGS

SEQ ID NO: 14 - protein, 27 aa, synthetic construct
  REGION 1..27: note = Synthetic
AAASAEAAAKEAAAKEAAAKAAAGSGL

SEQ ID NO: 15 - protein, 5 aa, synthetic construct
  REGION 1..5: note = Synthetic
GGGLG

SEQ ID NO: 16 - protein, 9 aa, synthetic construct
  REGION 1..9: note = Synthetic
RKKRRQRRR

SEQ ID NO: 17 - protein, 24 aa, Homo sapiens
MKWVTFISLLFLFSSAYSRGVFRR

SEQ ID NO: 18 - protein, 30 aa, Homo sapiens
MKRMVSWSFHKLKTMKHLLLLLLCVFLVKS

SEQ ID NO: 19 - protein, 461 aa, Homo sapiens
QGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 20 - protein, 26 aa, Homo sapiens
MSWSLHPRNLILYFYALLFLSSTCVA

SEQ ID NO: 21 - protein, 427 aa, Homo sapiens
YVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

(19) United States
(12) Patent Application Publication
(10) Pub. No.: US 2024/0350648 A1
(43) Pub. Date: Oct. 24, 2024

(54) INTRACELLULAR DELIVERY COMPOSITIONS

(71) Applicant: Biond Biologics Ltd., Misgav Industrial Park (IL)

(72) Inventors: Orit SHILOVITZKY, Haifa (IL); Lilach CHEN ZELTSBURG, Nahariya (IL); Rozi Ravit UZAN GUETA, Haifa (IL); Ayala LEWKOWICZ, Kibbutz Kfar Szold (IL); Yossi GOTTFRIED, Kiryat Tivon (IL); Rotem FAYGENBOIM-ORNAI, Tal-El (IL); Bat-Hen EYLON, Haifa (IL); Shay FREILICH, Kiryat Motzkin (IL); Motti HAKIM, Kibbutz Gazit (IL); Yair SAPIR, Manof (IL); Ilana MANDEL, Manof (IL); Tehila BEN-MOSHE, Tel Aviv (IL); Avidor SHULMAN, Rakefet (IL)

(21) Appl. No.: 18/653,316

(22) Filed: May 2, 2024

Related U.S. Application Data
(63) Continuation of application No. PCT/IL2022/051164, filed on Nov. 3, 2022.
(60) Provisional application No. 63/275,049, filed on Nov. 3, 2021.
(60) Provisional application No. 63/348,114, filed on Jun. 2, 2022.

(51) Int. Cl. A61K 47/64; A61K 47/54; A61K 47/59; A61K 47/60; A61K 47/68; A61K 49/00; A61P 35/00; C07K 1/107

(52) U.S. Cl. CPC A61K 47/643; A61K 47/542; A61K 47/59; A61K 47/60; A61K 47/64; A61K 47/6835; A61K 47/6889; A61K 49/0002; A61P 35/00; C07K 1/1077

(57) ABSTRACT

Protein conjugates comprising a protein carrier comprising a plurality of amine groups, a biological payload that interacts with an intracellular target and a linker linking them, wherein at least a portion of the amine groups are bound to a protecting group are provided. Pharmaceutical compositions comprising the protein conjugates as well as methods of using and producing the protein conjugates are also provided.

The contents of the electronic sequence listing (BDB-P-013-PCT2 SQL.xml; Size: 26,146 bytes; and Date of Creation: May 1, 2024) is herein incorporated by reference in its entirety.

### CROSS REFERENCE TO RELATED APPLICATIONS

This application is a Bypass Continuation of PCT Patent Application No. PCT/IL2022/051164 having International filing date of Nov. 3, 2022, which claims the benefit of priority of U.S. Provisional Patent Application No. 63/275,049, filed on Nov. 3, 2021, and U.S. Provisional Patent Application No. 63/348,114, filed on Jun. 2, 2022, the contents of which are hereby incorporated by reference in their entirety.

### FIELD OF INVENTION

The present invention is in the field of protein intracellular delivery.

### BACKGROUND OF THE INVENTION

In the search of new and potent therapeutic targets, especially in the field of oncology, it is very difficult to target intracellular targets, in particular targets that are deemed “undruggable”. Such targets cannot be targeted by small-molecule drugs, mainly because the required therapeutic intervention involves interfering with protein-protein interactions, where small molecule drugs have not been able to exhibit efficacy and/or sufficient selectivity. While such targets and biological processes are classically and efficiently addressed by biologic agents, e.g., monoclonal antibodies, receptors, interleukins, and their derivatives and combinations, such agents cannot easily enter living cells, especially cellular compartments that are clinically relevant, such as the cytoplasm, nucleus, endoplasmic reticulum (ER), etc. This is because the outer cell membrane is impermeable to protein-based molecules.

In cases where protein-based molecules are internalized to living cells by a natural endocytosis-based mechanism, this protein-based molecule will enter the endosomal pathway, which leads it to lysosomal degradation. Therefore, this route is naturally only relevant to the treatment of lysosomal disorders or to antibody-drug conjugates (ADC). In the former, the therapeutic protein-based agent is aimed to exert its activity in endosomes or lysosomes and is generally designed or is naturally suited to withstand the conditions of the endosome/lysosome. In the latter, the antibody is used purely for targeting/delivery of a small-molecule drug to specific cells, and lysosomal degradation of the antibody carrier releases the small-molecule drug, usually a cellular toxin.

However, if the protein is itself the therapeutic and not merely a targeting moiety, the technology of the invention should further enable what is known as “endosomal escape”. This crucial step frees the therapeutic biologic from the vesicles of the different stages of the endosomal pathway, i.e., early or late endosomes and lysosomes, in order to avoid catabolism of the therapeutic agent by this cellular machinery.

Following a successful escape from the endosomes, the therapeutic agent is released to the cell's cytoplasm. However, the cytoplasm is a crowded environment and not hospitable to most biologic therapeutic agents that are employed currently, e.g., monoclonal antibodies and their derivatives. The therapeutic biologic must be efficiently dispersed in the cytoplasm in order to locate and engage its therapeutic target or, alternatively, reach a target intracellular organelle, such as the nucleus, ER, mitochondria, etc.

One of the classical and most researched intracellular delivery techniques involves the use of enhanced positive charge. The most classical approach evolved from the understanding of intracellular uptake mechanisms employed by viruses. The latter employ positively charged peptides, rich with Arginine and Lysine amino acids, such as the famous HIV-derived TAT peptide (RKKRRQRRR (SEQ ID NO: 16)). This approach yielded numerous cell-penetrating peptides (CPPs) with different charges, amino acid sequences, additional modifications and structures (linear, cyclic, etc.) that were fused or chemically conjugated to a variety of payloads or used to decorate various nanoparticles. While CPPs exhibit the ability to internalize into cells, their endosomal escape efficiency is still debated, and their overall efficiency seems to be insufficient to be used in real pharmaceutical applications. An alternative method to make use of charge-based cell penetrance is to modify a biologic or a carrier with a highly positively charged polymer. One such polymer is polyethyleneimine (PEI).

Adequate PK and biodistribution profiles are crucial to ensure efficacy for any drug, especially a biologic agent. However, chemical modification with PEI or similar molecules characterized by a strong positive charge, may have a dramatic interfering effect on PK and biodistribution. As the majority of the circulatory proteins, as well as the lining of the blood vessels, are negatively charged, any positively charged protein introduced to the blood stream will adhere to those blood components, interfering with its PK and biodistribution. Furthermore, such adherence can also lead to “trapping” of the administered positively charged protein in the injection site. Utilizing the lowest level of modification may minimize the effects of the positive charge on PK and injection site “trapping” but further solutions are still required. As such, there is an unmet need to develop suitable charge masking groups which are sufficiently stable in the blood or in healthy tissues and undergo rapid and selective demasking/deprotection in the target tissue allowing cell penetration and intracellular delivery of biologic therapeutic agents.

### SUMMARY OF THE INVENTION

The present invention provides protein conjugates comprising a protein carrier comprising a plurality of amine groups, a biological payload that interacts with an intracellular target and a linker linking them, wherein at least a portion of the amine groups are bound to a protecting group. Pharmaceutical compositions comprising the protein conjugates as well as methods of using and producing the protein conjugates are also provided.

According to a first aspect, there is provided a protein conjugate comprising a biological payload that interacts with an intracellular target, wherein the biological payload is covalently bound to a cell penetrating moiety comprising a plurality of amine groups, at least a portion of the amine groups is bound to a protecting group and the protecting group is capable of undergoing cleavage at a pH value of less than 7; and wherein the protein conjugate is characterized by a negative zeta potential.

According to another aspect, there is provided a protein conjugate, comprising:
a. a protein carrier covalently bound to a cell penetrating moiety comprising a plurality of amine groups;
b. a biological payload that interacts with an intracellular target; and
c. a linker between the protein carrier and the biological payload; wherein:
at least a portion of the amine groups is bound to a protecting group;
the protecting group is capable of undergoing cleavage at a pH value of less than 7; and
the protein conjugate is characterized by a negative zeta potential.

According to some embodiments, the protein conjugate is characterized by an increased blood stability compared to an analogous protein conjugate devoid of the protecting group.

According to some embodiments, the protein conjugate is characterized by an increased accumulation within a biological tissue having a pH value of less than 7, compared to an analogous protein conjugate devoid of the protecting group.

According to some embodiments, the plurality of amine groups comprises a primary amine, a secondary amine, or both; and at least 50% of the plurality of amine groups are bound to the protecting group.

According to some embodiments, the linker is linked to the carrier, the payload or both by a covalent bond.

According to some embodiments, the protecting group comprises a moiety being negatively charged at a pH between 6 and 8.

According to some embodiments, the moiety comprises a carboxy group.

According to some embodiments, the protecting group is represented by Formula 1:

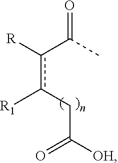

wherein n is an integer ranging from 0 to 5; ------ represents an attachment point to the amine group, and [symbol] represents a single bond or a double bond; R and R1 each independently represent a substituent selected from H, optionally substituted alkyl, optionally substituted cycloalkyl, optionally substituted aryl or heteroaryl, and carboxyalkyl), or any combination thereof; or R and R1 are bound together so as to form a cyclic ring.

According to some embodiments, one of R and R1 is H and another one of R and R1 comprises an alkyl or a carboxyalkyl.

According to some embodiments, the protecting group is

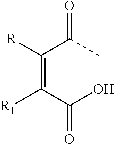

including any salt thereof, wherein R and R1 are selected from H and methyl, and wherein R or R1 is methyl.

According to some embodiments, the cell penetrating moiety comprises an alkyl amine, a cationic polymer, or a combination thereof.

According to some embodiments, the cationic polymer is selected from a polyamine and polyethyleneimine (PEI).

According to some embodiments, the PEI is a linear PEI or a branched PEI having a molecular weight of less than 2000 Daltons.

According to some embodiments, the PEI comprises a molecular weight of between 100 and 1000 Daltons.

According to some embodiments, the biological payload is an antigen binding molecule that binds the intracellular target.

According to some embodiments, the biological payload is devoid of a disulfide bond that when cleaved diminishes interaction with the intracellular target.

According to some embodiments, the antigen binding molecule is selected from a single chain antibody, a single domain antibody, a variable heavy homodimer (VHH), a nanobody, an immunoglobulin novel antigen receptor (IgNAR), a designed ankyrin repeat protein (DARPin) and an antibody mimetic protein.

According to some embodiments, the antigen binding molecule is selected from a VHH and a DARPin.

According to some embodiments, the protein carrier or biological payload comprises a plurality of PEI molecules. According to some embodiments, the protein carrier comprises between 2 and 30 PEI molecules.

According to some embodiments, the protein carrier is human serum albumin (HSA).

According to some embodiments, the HSA comprises between 3 and 10 PEI molecules.

According to some embodiments, the linker comprises a biocompatible polymer, a biodegradable polymer or both.

According to some embodiments, the biocompatible polymer comprises polyethylene glycol (PEG). According to some embodiments, the biodegradable polymer comprises a polyamino acid.

According to some embodiments, the linker further comprises a spacer covalently bound to (i) the biocompatible polymer or the biodegradable polymer and to (ii) the protein carrier. According to some embodiments, covalently bound is via a click reaction product.

According to some embodiments, the linker comprises a bio cleavable bond.

According to some embodiments, the bio cleavable bond comprises a disulfide bond.

According to some embodiments, the linker is substantially stable in blood for at least 24 hours.

According to some embodiments, the linker is a peptide linker.

According to some embodiments, stable comprises less than 25% cleavage in blood after 24 hours.

According to some embodiments, the bio cleavable bond is sterically hindered.

According to some embodiments, the HSA comprises the amino acid sequence of SEQ ID NO: 1, or a fragment or homolog thereof comprising cysteine 34 (C34).

According to some embodiments, the linker is bound to the HSA via a disulfide bond.

According to some embodiments, the linker is bound to the C34 of HSA.

According to some embodiments, the disulfide bond is proximal to the C34.

According to some embodiments, the proximal is at a distance from the C34 ranging from 5 to 15 angstroms.

According to some embodiments, the protein carrier is devoid of DNA.

According to some embodiments, the biological payload does not bind a cell surface protein.

According to some embodiments, the protein conjugate is characterized by a negative zeta potential of less than −1 mV.

According to some embodiments, the protein conjugate further comprises a detectable tag. According to some embodiments, the tag is conjugated to the biological payload.

According to some embodiments, the protein conjugate is a cell-penetrating conjugate.

According to some embodiments, the protecting group is citraconic anhydride.

According to some embodiments, the protecting group is derived from citraconic anhydride.

According to some embodiments, the click reaction product is succinimide-thioether.

According to some embodiments, the protein conjugate further comprises a targeting moiety that binds to a protein expressed on the surface of a target cell.

According to some embodiments, the targeting moiety is selected from a single chain antibody, a single domain antibody, a variable heavy homodimer (VHH), a nanobody, an immunoglobulin novel antigen receptor (IgNAR), a designed ankyrin repeat protein (DARPin) and an antibody mimetic protein.

According to some embodiments, the targeting moiety is conjugated to the protein carrier via a linker.

According to some embodiments, the targeting moiety and the biological payload are comprised in a single polypeptide. According to some embodiments, the targeting moiety and the biological payload are separated by a linker.

According to some embodiments, the targeting moiety is N-terminal to the biological payload or the biological payload is N-terminal to the targeting moiety.

According to another aspect, there is provided a method of producing a charge masked protein conjugate capable of binding an intracellular target, the method comprising:
a. providing a biological payload that binds the intracellular target, wherein the biological payload is covalently bound to a cell penetrating moiety comprising a plurality of amine groups; and
b. providing the biological payload under conditions sufficient for protecting at least a portion of the amine groups by a protecting group capable of undergoing cleavage at a pH value of less than 7, to obtain the charge masked protein conjugate comprising protected amine groups;
thereby producing a charge masked protein conjugate capable of binding an intracellular target.

According to another aspect, there is provided a method of producing a charge masked protein conjugate capable of binding an intracellular target, the method comprising:
a. providing a biological payload that binds the intracellular target;
b. providing a protein carrier covalently bound to a cell penetrating moiety comprising a plurality of amine groups;
c. providing the biological payload and the protein carrier under conditions sufficient for covalently binding the biological payload to the protein carrier via a linker to produce a protein conjugate; and
d. providing the protein conjugate under conditions sufficient for protecting at least a portion of the amine groups by a protecting group capable of undergoing cleavage at a pH value of less than 7, to obtain the charge masked protein conjugate comprising protected amine groups;
thereby producing the charge masked protein conjugate capable of binding an intracellular target.

According to some embodiments, the method further comprises determining stability of the linker in human blood, plasma or serum and in cytoplasmic conditions; and selecting a charge masked protein conjugate comprising a linker that is stable in the human blood, plasma or serum and unstable in the cytoplasmic conditions.

According to some embodiments, the method further comprises determining stability of the protected amine groups at neutral or basic pH and at acidic pH and selecting a charge masked protein conjugate comprising protected amine groups that are stable at neutral or basic pH and unstable at acidic pH.

According to some embodiments, the providing the protein carrier under conditions sufficient for protecting occurs before the binding the biological payload to the protein carrier.

According to some embodiments, the providing the protein carrier under conditions sufficient for protecting occurs after the binding the biological payload to the protein carrier.

According to some embodiments, the determining is performed before formation of the protein conjugate or after formation of the charge masked protein conjugate.

According to some embodiments, the protein carrier comprises HSA.

According to some embodiments, the cell penetrating moiety comprises at least one PEI.

According to some embodiments, the charge masked protein conjugate is characterized by a negative zeta potential.

According to some embodiments, the plurality of amine groups comprises a primary amine, a secondary amine, or both; and at least 80% of the plurality of amine groups are protected amine groups.

According to some embodiments, the protecting group comprises a moiety being negatively charged at a pH between 6 and 8.

According to some embodiments, the moiety comprises a carboxy group.

According to some embodiments, the protein carrier or biological payload is covalently bound to at least 2 molecules of PEI. According to some embodiments, the protein carrier is covalently bound to at least 8 molecules of PEI.

According to some embodiments, the biological payload is devoid of a disulfide bond that when cleaved diminishes binding to the intracellular target.

According to some embodiments, the method further comprises contacting the charged masked protein conjugate with a cell and confirming the biological payload enters a cytoplasm of the cell.

According to some embodiments, stable comprises less than 25% cleavage in blood after 24 hours and unstable comprises at least 50% cleavage in the cytoplasmic conditions after 24 hours.

According to some embodiments, the linker comprises a biocompatible polymer.

According to some embodiments, the covalently linking is via a click reaction.

According to some embodiments, the biological payload is covalently bound to a linker comprising a first reactive group; and wherein the protein carrier is covalently bound to a linker comprising a second reactive group having reactivity to the first reactive group; and wherein the conditions sufficient for covalently binding the biological payload to the protein carrier comprises reacting the first reactive group with the second reactive group, thereby covalently linking the biological agent and the protein carrier.

According to some embodiments, the linker comprises a bio cleavable bond.

According to some embodiments, the covalently linking comprises disulfide bond formation.

According to some embodiments, (i) the biological payload is covalently bound to a linker capable of generating a disulfide bond with a cysteine of the protein carrier; or (ii) the protein carrier is covalently bound to a linker capable of generating a disulfide bond with a cysteine of the biological payload. According to some embodiments, bound is via a disulfide bond.

According to some embodiments, the method further comprises selecting a targeting moiety that binds to a protein expressed on the surface of a target cell and conjugating the targeting moiety to the biological payload, the protein carrier or the protein conjugate.

According to some embodiments, the targeting moiety is selected from a single chain antibody, a single domain antibody, a variable heavy homodimer (VHH), a nanobody, an immunoglobulin novel antigen receptor (IgNAR), a designed ankyrin repeat protein (DARPin) and an antibody mimetic protein.

According to some embodiments, the targeting moiety and the biological payload are comprised in a single polypeptide. According to some embodiments, the targeting moiety and the biological payload are separated by a linker.

According to some embodiments, the targeting moiety is N-terminal to the biological payload or the biological payload is N-terminal to the targeting moiety.

According to some embodiments, the charge masked protein conjugate is the protein conjugate of the invention.

According to another aspect, there is provided a protein conjugate produced by a method of the invention.

According to another aspect, there is provided a pharmaceutical composition, comprising the protein conjugate of the invention and a pharmaceutically acceptable carrier, excipient or adjuvant.

According to some embodiments, the pharmaceutical composition is formulated for systemic administration.

According to another aspect, there is provided a method of binding an intracellular target, the method comprising contacting a cell expressing the intracellular target with the protein conjugate of the invention or the pharmaceutical composition of the invention, wherein the biological payload binds the intracellular target, thereby binding the intracellular target.

According to some embodiments, the method is a method of detecting an intracellular target and the protein conjugate comprises a detectable tag, and wherein the method further comprises detecting the detectable tag.

According to some embodiments, the method is a method of modulating the intracellular target and wherein the biological payload is an agonist or antagonist of the intracellular target.

According to some embodiments, the cell is in a subject and wherein the contacting comprises administering the protein conjugate the invention or a pharmaceutical composition of the invention to the subject.

According to some embodiments, the cell expresses a target surface protein and the protein conjugate comprises a targeting moiety that binds to the target surface protein.

According to some embodiments, the method is a method of treating a condition in a subject in need thereof, wherein the condition is treatable by modulation of the intracellular target.

According to some embodiments, the condition comprises cancer or inflammation.

According to some embodiments, the condition is cancer, the intracellular target is oncogenic and the biological payload is an antagonist.

According to some embodiments, the cancer comprises a target surface protein that is a cancer specific antigen.

According to some embodiments, the contacting is not in the presence of an agent other than the carrier protein designed to induce penetration of the protein conjugate to the cell.

According to some embodiments, the method is for delivering biological payload to a specific tissue within the subject, wherein the specific tissue is characterized by a pH value of below 7. According to some embodiments, the specific tissue is a tumor.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: MALDI-ToF spectra of non-modified and PEI-modified mouse IgG at various modification levels.

FIG. 2: Micrographs of internalization of PEI-modified or unmodified mouse IgG into A375 cells (Blue—nuclei, Green—stained mouse IgG). Native IgG is not modified, and all other panels show ascending levels of modification with PEI. The numbers in the corner of each panel denote the average number of PEI molecules per IgG molecule.

FIGS. 3A-3B: Micrographs of internalization of PEI-modified GFP (average of 6.5 PEIs per GFP molecule) into (3A) HEK cells (60 ug/mL) and (3B) A375 cells (5 ug/mL).

FIG. 4: Bar chart of cellular uptake efficiency of PEI-modified IgG (initial media concentration of 2 μg/mL) at various levels of modification, as measured by a specific ELISA of IgG levels in the media.

FIG. 5: Histograms showing inhibition of Caveolin-mediated endocytic internalization of PEI-modified IgG (4.5 PEIs). NC—non-modified IgG.

FIG. 6: Micrographs of staining of HEK293 cells, expressing an endosomal marker (upper panels) or a lysosomal marker (lower panels) following their incubation (5 hours) with 5 μg/mL PEI-modified IgG (3×PEI, left panels; 4.5×PEI, middle panels and 7×PEI, right panels). Endosomal and lysosomal markers appear in Green, PEI-modified IgG in Red and nuclei are stained in Blue.

FIG. 7: Micrograph of endosomal escape monitoring using live imaging confocal microscopy of PEI-modified IgGs (4.5×PEI) labeled with a pH sensitive dye (5(6)-carboxynaphthofluorescein, Red) following overnight incubation of the PEI-modified IgG at 10 μg/mL with 2×10⁴ HeLa cells. Green—Tubulin staining, Blue—nuclei staining.

FIG. 8: Bar chart of CD3 cells activation as reflected by dose dependent increase in IFNγ secretion following internalization of anti-CD247 PEI-modified mAb (4.5×PEI). No changes in IFNγ levels were observed for internalized PEI-modified mouse IgG at the evaluated concentration range.

FIG. 9: A schematic representation of the carrier and payload methodology in which a PEI-cationized carrier protein is chemically linked to a therapeutic agent, an antibody in this example, where the linker is conjugated to the Fc region of the antibody, away from its CDRs, and the linker includes a disulfide bond that can be reduced and cleaved in the cytoplasm, releasing the therapeutic payload from the cationized carrier enabling free cytosolic trafficking.

FIG. 10: Micrographs showing the intracellular profile of GFP following the incubation of A375 cells with GFP conjugated by PEG-based disulfide containing linker to PEI-modified HSA (7×PEI). Cells exhibit a dispersed GFP profile at 24 (left) and 48 (right) hours post incubation as observed by confocal microscopy (Green—GFP; Blue—nuclei).

FIG. 11: Micrographs of anti-TNFα antibody conjugated to PEI-modified HSA (11×PEI) via a disulfide-bond containing PEG linker following overnight incubation with A375 cells and as detected by confocal microscopy using a fluorescent labeled (AlexaFluor 647) anti-human IgG antibody.

FIG. 12: SDS-PAGE Western blot analysis of full mouse IgG in PBS 7.4 (lanes 8, 9), after 4 hours in PBS containing 10 mM of GSH (lanes 5-7) and overnight after 4 hours (lanes 1-4). The gel was treated with anti-mouse light chain antibody.

FIG. 13: Exemplary results of the production, purification and characterization of the VHH-PEI-modified HSA construct generated by modifying HSA with PEI followed by modification of the HSA with NHS-PEG₄-SPDP. The SPDP modified HSA-PEI is further reacted with a VHH with a C-terminal free Cysteine. The latter can be optionally pre-treated with a reducing agent (TCEP for example) to make sure all Cysteine is free. Lane 1—HSA-8×PEI, lane 2—HSA-8×PEI—S-S-VHH (arrows point to the different reaction products), lane 3—Reaction products following reduction with DTT (suggesting the bond between the HSA and VHH was a labile disulfide bond), lanes 7-9-fractions of the VHH-HSA conjugate following purification on a Protein A resin (removal of excess HSA-8×PEI). MS spectra show the profile of the products of VHH-HSA-0×PEI (upper), VHH-HSA-4×PEI (middle) and VHH-HSA-8×PEI (bottom), all showing the major product to comprise an HSA attached to a single VHH.

FIG. 14: Bar chart of media levels of anti-Vimentin VHH conjugated to PEI modified HSA with an average of 3.5 (blue) or 8 (red) PEI molecules per HSA in the presence of A375 cells (dark shades) or in their absence (light shades).

FIGS. 15A-15C: Micrographs of confocal microscopy imaging of A375 cells stained for (15A) VHH, (15B) Vimentin and (15C) a merge of both following 24 hours of incubation with anti-Vimentin VHH reversibly conjugated to PEI-modified HSA. Nuclei are stained in blue.

FIGS. 16A-16B: Micrographs of confocal microscopy imaging of A375 cells stained for VHH (Green) following 24 hours of incubation with (16A) anti-Vimentin VHH reversibly conjugated to PEI-modified HSA or (16B) unconjugated anti-Vimentin VHH. Nuclei are stained in blue.

FIG. 17: Bar chart HeLa proliferation index. HeLa (HPV+) cells were incubated for 48 hours in medium (Black bar) or medium supplemented with anti-E7 VHH (Grey bar) or anti-E7 VHH conjugated to PEI-modified HSA (average of 3.5 PEIs per HSA) at various concentrations (Green bars). HeLa cells proliferation following incubation was measured by a standard MTT assay.

FIG. 18: Line graph showing the percent of HPV-positive HeLa FUCCI cells in the S, G2 and M cell cycle stages following Thymidine cell cycle synchronization. Cells were treated with anti-E7 VHH conjugated to HSA modified with an average of 3.5 PEIs, and various control treatments. The controls were: no treatment (wo), the unmodified VHH anti-E7, anti-E7 VHH conjugated to unmodified HSA, modified HSA conjugated to an irrelevant VHH (anti-Vimentin), the modified HSA carrier alone, and a cell cycle inhibitor (DP, CDK4/6 inhibitor).

FIG. 19: Micrographs of the effect of different treatments on HPV-positive HeLa FUCCI cells. Cells treated with an irrelevant VHH (anti-Vimentin) conjugated to HSA modified with an average of 3.5 PEIs show the same profile as untreated cells (left images) while an anti-E7 VHH conjugated to the same HSA carrier caused dramatic cell death, similar to that observed following treatment of the CDK4/6 inhibitor (right images).

FIG. 20: Representative micrograph of anti-K-RAS His-tagged DARPin K27 protein conjugated to a PEI-modified HSA carrier internalized into lung adenocarcinoma cells. The DARPin localizes to K-RAS sites (inner-side of cell membrane) following staining with anti-His tag antibody.

FIG. 21: Line graph of apoptosis. Anti-KRAS DARPin K27, conjugated to the PEI-modified HSA carrier was internalized to SU8686 cells and their apoptotic state was evaluated using Annexin V using the continuous Incucyte system. Cells exposed to the DARPin conjugated to a carrier modified with 8 PEI molecules (Carrier-I) exhibit a high level of apoptosis while cells exposed to the DARPin conjugated to the carrier with 3.5 PEI molecules (Carrier II) also exhibit clear apoptosis but to a lesser degree. Untreated cells, cells exposed to the unmodified DARPin and cell exposed to the HSA carrier with 8 PEIs all exhibit baseline apoptosis.

FIGS. 22A-22B: Line graphs showing the effect of anti-KRAS DARPin K27 conjugated to PEI-modified HSA carrier on (22A) proliferation and (22B) apoptosis of HeLa cells constitutively expressing GFP in their nuclei. A pan-RAS inhibitor was used as a positive control and no treatment was used as a negative control. Cells were also treated with the unmodified DARPin K27, the PEI-modified HSA carrier alone, DARPin K27 conjugated to an unmodified HSA carrier and an anti-vimentin VHH conjugated to PEI-modified HSA carrier.

FIG. 23: Micrographs showing the effect of anti-KRAS DARPin K27 conjugated to PEI-modified HSA carrier on apoptosis of HeLa cells constitutively expressing GFP in their nuclei.

FIG. 24: Mass spectrometry (MALDI-ToF) spectra of HSA modified with various levels of PEI (600 Da) using a constant molar excess of the PEI in the reaction and controlling the level of modification by adjusting the levels of the carbodiimide coupling agent, EDC. The average level of PEI molecules on the HSA is provided in black.

FIG. 25: Confocal microscopy images of anti-Vimentin VHH inside A375 cells following 48 hours of incubation of the VHH conjugated to the HSA carrier with various levels of PEI modification. The average number of PEI molecules on the HSA carrier is denoted in each box. Intracellular VHH is visualized using an anti-VHH antibody.

FIG. 26: Bar graph of residual levels of VHH-carrier conjugates with either 8 (Orange) or 3.5 (Blue) PEI molecules per HSA remaining in the media as measured by specific ELISA in the media of A375 cells incubated with the conjugates.

FIGS. 27A-27B: IVIS images of (27A) whole mice and (27B) harvested organs from mice that received one of two doses of unmasked carrier or masked carrier. Relative signal intensity was calculated as radiant efficiency (Emission light [photons/sec/cm2/str]/Excitation light [μW/cm2]×109) per pixel of the region of interest (27A—all animal live imaging; 27B—respective organs ex vivo imaging). Relative signal intensity is presented as a color scale. Colors represent injected item localization and concentration, with yellow color indicating increased fluorescence intensity and dark red color indicating reduced fluorescence intensity.

FIGS. 28A-28F: Confocal microscopy images of Hela GFP cells following 24 hr. incubation with: (28A-28C) anti-E7-HSA-PEI×3.5, or (28D-28F) α-E7-masked-HSA-PEI×3.5. Immunofluorescence staining using anti-VHH antibodies—Red; Nuclear staining (GFP)—green. (28A, D)—nuclear staining; (28B, E)—VHH staining; (28C, F)—Merge. Intracellular VHH staining is observed only with the unmasked conjugate. Intracellular VHH staining is observed predominantly in the cytoplasm.

FIG. 29A-29F: Confocal microscopy images of B16 cells following 24 hr. incubation with: (2A) HSA-PEI×8; (2B) HSA-PEI×8-CA or (2C) HSA-PEI×8-MSA, or (2D) HSA-PEI×8; (2E) HSA-PEI×8-CA or (2F) HSA-PEI×8-MSA that were pretreated at acidic conditions for masking removal before incubation with the B16 cells. Carrier staining—Red; Nuclear staining (DAPI)—Blue.

FIGS. 30A-30B: (30A) Percentage of living MEL-526 cells following treatment with either 1C5-non-masked HSA-PEI×3.5 or 1C5-masked HSA-PEI×3.5 before and after 8 hours of masking removal. Calculation was done in comparison to cells without treatment. Cells were challenged with the respected agents at 6 μM for six days. (30B) Percentage of living SK-MEL-28 cells following treatment with either 1C5-masked HSA-PEI×3.5, 1C5-masked HSA-PEI×3.5 after removal (1 hour removal process), 1C5-HSA-PEI×3.5, or without treatment. Cells were challenged with the respected agent at 10 μM for six days.

FIGS. 31A-31B: (31A) Line graph showing pharmacokinetics of modified HSA (8/3.5 PEI units) with and without masking. Lines represent mean±SEM, of log HSA plasma concentration [μg/mL], (n=3). Two tailed Student's t-test yields significant differences between Masked HSA-PEI×8 vs. HSA-PEI×8 and HSA-PEI×3.5 *p<0.01** (31B) Bar graph representing the plasma exposure of HSA-PEI derivatives in vivo.

FIGS. 32A-32B: (32A) Bar graph of exposure level in various organs of HSA-PEI derivatives in vivo. (32B) Bar graph of the biodistribution of Masked HSA-PEI×8, at 1440 min after IV injection. Bars represent mean±SEM, of HSA concentration [μg/mL], (n=3). One way ANOVA test yields a significant difference in the biodistribution between organs. Post-hoc Dunnett indicated a significance of p<0.0001 (****) between tumor HSA concentration as compared to each of the organs tests. Similar results were obtained for other time points.

FIG. 33: Plot of HSA-PEI derivatives found in urine.

FIG. 34: Line graph showing pharmacokinetics of modified IgG (4 PEI units) with and without masking.

FIG. 35: Line graph showing pharmacokinetics of directly modified VHH (PEI 1800) with and without masking.

FIGS. 36A-36D: (36A) Line graphs of average tumor volume in mice inoculated with HeLa cells and treated with 350 nmol/Kg of the αE7-VHH-S-Mal-PEG₁₁-Mal-S-HSA×3.5 conjugate or αE7-VHH-S-Mal-PEG₁₁-Mal-S-HSA or just vehicle (PBS), every day for 15 days. (36B-36C) Line graphs of tumor volume in mice inoculated with HeLa cells and treated with (36B) 250 nmol/Kg of the αE7-VHH-S-Mal-PEG₁₁-Mal-S-HSA×3.5 citraconic anhydride masked conjugate, the masked carrier alone or PBS every other day for 5 days followed by 5 daily injections or (36C) 350 nmol/Kg of the αE7-VHH-S-Mal-PEG₁₁-Mal-S-HSA×3.5 masked conjugate, the masked carrier alone or PBS every day for 15 days. (36D) Bar graph of percent tumor inhibition by the masked conjugate as compared to the masked carrier control over the course of the experiment.

FIGS. 37A-37B: Micrographs of immunohistochemical detection of (37A) the VHH payload and (37B) HSA modified PEI carrier in tumor sections from mice treated with PBS (left), citraconic anhydride masked PEI modified carrier (middle), and masked conjugate of the invention (right).

FIGS. 38A-38B: Line graphs of tumor volume in mice inoculated with (38A) B16 cells or (38B) MEL-526 cells and treated with 350 nmol/Kg of the 1C5-VHH-S-Mal-PEG₁₁-Mal-S-HSA-PEI×3.5 masked with citraconic anhydride conjugate (αBRAF (1C5)-Masked Carrier 3.5) or the masked carrier alone for 15 days of daily IV injections. * p-value <0.05.

FIG. 39: Line graph of the pharmacokinetic profiles of different conjugates of anti-E7 VHH conjugated to masked carriers, differing in the level of PEI and the reversibility of the payload-carrier bond or the reversibility of the masking.

FIG. 40: Bar graph of the biodistribution of the different masked conjugates in various organs, including tumor, presented as organs exposure levels (AUC).

FIGS. 41A-41E: (41A-41B) Confocal microscopy images (X63) of tumor tissue from athymic nude Foxn1nu mice bearing cervical tumors (HeLa-GFP), 6 hours after injection of (41A) Atto 542-HSA^{PEI×8} CA and (41B) Atto 542-HSA^{PEI×8} MSA. Carrier signal (Red); Nuclear staining (Blue). (41C-41E) Confocal microscopy images (X63) of tumor tissue from C57BL mice bearing B16 tumors 6 hours after injection of (41C) Atto 542-HSA^{PEI×8} CA, (41D) Atto 542-HSA^{PEI×8} MSA and (41E) Atto 542-unmasked HSA^{PEI×8}. Carrier signal (Red); Nuclear staining (Blue).

FIGS. 42A-42D: (42A-42B) Confocal microscopy images (X40) of tumor tissue from athymic nude Foxn1nu mice bearing cervical tumors (HeLa-GFP), 6 hours after injection of Atto 542-HSA-PEI×8 MSA. Carrier staining (Red); Nuclear staining (Blue); Nuclear staining from green fluorescent protein in the tumor (Green). (42A) Staining of the carrier (Red) and tumor cells nuclei (Green). (42B) Staining of the carrier (Red) and general cell nuclei staining (Blue). (42C-42D) Confocal microscopy images (X63) of liver tissue from C57BL mice bearing B16 tumors, 6 hours after injection of (42C) Atto 542-HSA^{PEI×8} CA and (42D) Atto 542-HSA^{PEI×8} MSA. Carrier signal (Red); Nuclear staining (Blue).

FIGS. 43A-43B: (43A) Bar graph of BRAF binding by masked anti-BRAF VHH, 1C5, before and after masking removal. (43B) Bar chart of the percentage of living MEL-526 cells following treatment with either 1C5-Hel-non-masked HSA-PEI×8 or fully masked 1C5-Hel-HSA-PEI×8 before and after 8 hours of masking removal. Calculation was done in comparison to cell without treatment. Cells were challenged with the respected agent at 6 μM for six days.

FIG. 44: Bar graph representing the percent of internalization of HSA-PEI×3.5 at different masking levels compared to the level of internalization of the HSA-PEI×3.5 without masking.

FIGS. 45A-45B: Bar graph summarizing FACS data of binding to PSMA positive and negative cells of tandem agents containing an anti-PSMA targeting moiety (45A) without and (45B) with the masked carrier.

FIG. 46: Line graph of binding of agents containing anti-PSMA targeting moiety to BRAF.

FIG. 47: Bar graph of cytotoxic effect of anti-BRAF VHH alone or expressed in tandem with anti-PSMA VHH on cells following conjugation to a carrier (3.5 PEIs) as measured by the Cell Titer Glo viability assay.

### DETAILED DESCRIPTION OF THE INVENTION

The present invention, in some embodiments, provides a protein carrier covalently bound to a cell penetrating moiety; wherein the cell penetrating moiety comprises a plurality of amine groups; at least a portion of the amine groups is bound to a protecting group; the protecting group is stable at a pH value of above 7, and is capable of undergoing disassociation from the portion of the amine groups at a pH value of less than 7. In some embodiments, the protected protein carrier is characterized by a negative zeta potential.

The invention is based on the discovery of a transient masking technology suitable for targeted intracellular delivery. This technology enables the masking of positive charges of a therapeutic agent or a carrier for the minutes or, preferably, hours post injection allowing “injection site escape” as well as enough time for the carrier and its payload to circulate in the blood and to reach target sites. The charge masking is based on covalent masking. This approach is similar to pro-drugs, in which a “problematic” group on a drug molecule is covalently substituted so the nature of the original group is changed, i.e., its polarity, solubility or charge. The substitution is designed to be unstable under general physiological conditions or under specific conditions, such as specific pH or in the presence of a specific enzyme. The unstable substitution thus gets removed in the target conditions leaving the positively charged molecule to be internalized and the payload delivered to the cytoplasm.

In a first aspect, there is provided a protein conjugate, comprising a protein carrier covalently bound to a biological payload that interacts with an intracellular target. In some embodiments, the protein conjugate of the invention is a charge masked conjugate.

In another aspect, there is provided a biological payload that interacts with an intracellular target bound to a cell penetrating moiety and a protecting group. In some embodiments, the biological payload, cell penetrating moiety and protecting group are a protein conjugate. In some embodiments, the biological payload, cell penetrating moiety and protecting group are comprised in a composition.

In another aspect, there is provided a protein conjugate, comprising a protein carrier covalently bound to a cell penetrating moiety; a biological payload that interacts with an intracellular target; and a linker between the protein carrier and the biological payload; wherein: the biological payload is devoid of a disulfide bond that when cleaved diminishes interaction with the intracellular target; the linker comprises a bio cleavable bond; the cell penetrating moiety comprises a plurality of amine groups, and at least a portion of the amine groups is bound to a protecting group; and wherein the protecting group is stable at a pH value of above 7, and is capable of undergoing disassociation from portion of the amine groups at a pH value of less than 7. In some embodiments, at least a portion of the amine groups are bound to a protecting group so as to result in protected amines, wherein a molar ratio of the protected amines to unprotected amines is so that the protein conjugate of the invention is characterized by a negative zeta potential of at least −0.1 mV, at least −0.5 mV, at least −1 mV, at least −2 mV, at least −3 mV, at least −5 mV, between −0.1 and −50 mV, between −0.5 and −50 mV, or between −0.5 and −30 mV, including any range between. In some embodiments, a molar ratio of the protected amines to unprotected amines in the protein conjugate of the invention is at least about 7:10, at least about 8:10, at least about 9:10, at least about 1:1, or between about 1:1 and 100:1, including any range between.

In another aspect, there is provided a protein conjugate, comprising a protein carrier covalently bound to a cell penetrating moiety; a biological payload that interacts with an intracellular target; and a linker between the protein carrier and the biological payload; wherein: the cell penetrating moiety comprises a plurality of amine groups; wherein: (i) at least about 30%, at least about 40%, at least about 50%, between about 40 and about 95%, between about 40 and about 100%, between about 40 and about 70%, between about 40 and about 80%, between about 40 and about 90%, between about 40 and about 95%, or between about 40 and about 99%, of the amine groups is bound to a protecting group, including any range between, (ii) the biological payload is bound to one or more protecting groups, or both (i) and (ii); and wherein the protecting group is stable at a pH value of above 7 (e.g. between 7.0 and 10, or between 7.2 and 10), and is capable of undergoing cleavage at a pH value of less than 7 (e.g., between about 5 and about 6.8, between about 3 and about 6.8, between about 5 and 7.0, between about 3 and 7.0, including any range between), and wherein the protein conjugate is characterized by a negative zeta potential.

In another aspect, there is provided a protein conjugate, comprising a protein carrier covalently bound to a cell penetrating moiety; a biological payload that interacts with an intracellular target; and a linker between the protein carrier and the biological payload; wherein: the cell penetrating moiety comprises a plurality of amine groups; wherein: (i) at least about 40%, or at least about 50% of the amine groups is bound to a protecting group, (ii) the biological payload is bound to one or more protecting groups, or both (i) and (ii); and wherein each protecting group is independent represented by Formula 2; and wherein the protein conjugate is characterized by a negative zeta potential of at least −0.1 mV, at least −0.5 mV, at least −1 mV, at least −2 mV, at least −3 mV, at least −5 mV, between −0.1 and −50 mV, between −0.5 and −50 mV, between −0.5 and −30 mV, including any range between.

In another aspect, there is provided a protein conjugate, comprising a protein carrier covalently bound to a cell penetrating moiety; a biological payload that interacts with an intracellular target; and a linker between the protein carrier and the biological payload; wherein: the cell penetrating moiety is or comprises one or more PEI molecules (e.g. between 3 and 10 PEI molecules per single protein carrier); wherein: (i) the one or more PEI molecules is bound to one or more protecting groups, so that the protein conjugate is characterized by a negative zeta potential, (ii) the biological payload is bound to one or more protecting groups, or both (i) and (ii); and wherein the protecting group is derived from citraconic anhydride.

In another aspect, there is provided a protein conjugate, comprising a protein carrier covalently bound to a cell penetrating moiety; a biological payload that interacts with an intracellular target; and a linker between the protein carrier and the biological payload; wherein: the cell penetrating moiety is or comprises one or more PEI molecules (e.g. between 3 and 10, or between 4 and 10 PEI molecules per single protein carrier); wherein: (i) at least about 40%, at least about 50%, between about 40 and about 95%, between about 40 and about 100%, between about 40 and about 70%, between about 40 and about 80%, between about 40 and about 90%, between about 40 and about 95%, or between about 40 and about 99% of amine groups of the one or more PEI molecules are covalently bound to a protecting group, so that the protein conjugate is characterized by a negative zeta potential, (ii) the biological payload is covalently bound to one or more protecting groups, or both (i) and (ii); and wherein the protecting group is derived from citraconic anhydride.

By another aspect, there is provided a protein conjugate, comprising a protein carrier covalently bound to a cell penetrating moiety; a biological payload that interacts with an intracellular target; and a linker between the protein carrier and the biological payload; wherein the protecting group undergoes cleavage at a pH of less than 7; and wherein the protein conjugate is characterized by a negative zeta potential.

In some embodiments, the linker is bound to the protein carrier and the biological payload. In some embodiments, the linker is linked to the carrier by a covalent bond. In some embodiments, the linker is bound to the carrier by a covalent bond. In some embodiments, the linker is linked to the payload by a covalent bond. In some embodiments, the linker is bound to the payload by a covalent bond. In some embodiments, the linker comprises a bond. In some embodiments, the linker is a bond. In some embodiments, the linker is a flexible linker. In some embodiments, the linker is a rigid linker. In some embodiments, the linker is of sufficient length to not cause steric hinderance between the payload and the carrier. In some embodiments, the linker is of sufficient length to allow access to the bio-cleavable bond. In some embodiments, access is access by the agent that cleaves the bio-cleavable bond. In some embodiments, the agent is an enzyme. In some embodiments, the agent is a reactive species. In some embodiments, the agent is a reducing agent.

In some embodiments, the linker comprises a length of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 atomic bonds. Each possibility represents a separate embodiment of the invention. As used herein, the term “atomic bond” refers to carbon-carbon (C—C) bond length, e.g., a single C—C bond length. In some embodiments, the linker comprises a length of at least 2, 4, 5, 6, 8, 10, 15, 20, 25, 30, 35, 40, 45, 50, 55, 60, 65, 70, 75, 80, 85, 90, 95 or 100 angstroms (Å). Each possibility represents a separate embodiment of the invention.

In some embodiments, the linker comprises a bio-cleavable bond. In some embodiments, the covalent bond is a bio-cleavable bond. In some embodiments, the linker is a bio-cleavable bond. In some embodiments, the linker is bound to the payload by a bio-cleavable bond. In some embodiments, the linker is bound to the payload by a bio-cleavable bond. In some embodiments, the protein conjugate is characterized by a negative zeta potential. In some embodiments, the protein conjugate is configured to release the biological payload within a cell. In some embodiments, the protein conjugate is configured to release the biological payload in the cytosol.

In some embodiments, the linker is sufficiently long such that the carrier does not interfere with the function of the payload. In some embodiments, the linker is sufficiently long such that the carrier does not interfere with payload binding. In some embodiments, binding is binding to an intracellular target. In some embodiments, the linker is sufficiently long such that the cell penetrating moiety does not interfere. In some embodiments, the linker is sufficiently long such that the carrier does not create steric hindrance to the payload.

In some embodiments, the protein carrier is a protein with a long serum half-life. In some embodiments, the protein carrier is a protein found in blood. In some embodiments, the carrier protein comprises a molecular weight of at least 60 kDa. In some embodiments, the carrier protein comprises a molecular weight of at least 65 kDa. In some embodiments, the carrier protein comprises a molecular weight of at least 70 kDa. In some embodiments, the carrier protein comprises a molecular weight of less than 200, 190, 180, 170, 160, 150, 140, 130, 120, 110, 100, 95, 90, 85, 80, 75, or 70 kDa. Each possibility represents a separate embodiment of the invention. In some embodiments, the carrier protein comprises an isoelectric point of at most 7. In some embodiments, the protein carrier is a human protein. In some embodiments, the protein carrier is albumin. In some embodiments, the albumin is serum albumin. In some embodiments, the serum albumin is human serum albumin (HSA). In some embodiments, the protein carrier is human serum albumin (HSA).

In some embodiments, the protein carrier is covalently bound to one or more cell penetrating moieties. In some embodiments, the protein carrier is covalently bound to a single cell penetrating moiety. In some embodiments, the protein carrier is covalently bound to a plurality of cell penetrating moieties. In some embodiments, the biological payload is covalently bound to one or more cell penetrating moieties. In some embodiments, the biological payload is covalently bound to a single cell penetrating moiety. In some embodiments, the biological payload is covalently bound to a plurality of cell penetrating moieties.

In some embodiments, the cell penetrating moiety comprises a cell-internalizing molecule. In some embodiments, the cell penetrating moiety is configured to internalize the protein conjugate of the invention into the cell. In some embodiments, the cell penetrating moiety is configured to induce or enhance cellular internalization of the protein conjugate of the invention. In some embodiments, the cell penetrating moiety is configured to enhance cell penetration or internalization of the protein conjugate of the invention, compared to a control (e.g., protein conjugate without the cell penetrating moiety). In some embodiments, cell-internalization comprises plasma membrane crossing. In some embodiments, cell-internalization comprises delivery to the cytosol. In some embodiments, cell-internalization comprises delivery to the cytoplasm. In some embodiments, cell-internalization comprises endosomal escape.

In some embodiments, enhance is by at least 20%, at least 50%, at least 100%, at least 1000%, at least 10000%, at least 100000%, including any range between, compared to a control. Each possibility represents a separate embodiment of the invention.

In some embodiments, dissociation is unbinding. In some embodiments, dissociation is cleavage of the PG. In some embodiments, the protecting group is capable of undergoing cleavage at a pH value of less than about 7. In some embodiments, the protecting group is cleaved at a pH value of less than about 7 (e.g., between 0 and about 7, between 3 and about 7, between about 5 and about 7, between about 5 and about 6.8, between about 3 and about 7, including any range between). In some embodiments, at least 50%, at least 70%, at least 80%, at least 90%, at least 95% of the protecting groups undergo cleavage at a pH value of less than about 7 (e.g. between 0 and about 7, between about 3 and about 7, between about 5 and about 7, between about 5 and about 7, including any range between), within a time period of up to 0.1 h, up to 0.5 h, up to 1 h, including any range between). A skilled artisan will appreciate that some cleavage of the protecting groups may also occur at higher pH values (with low reaction kinetics, thus requiring long time periods to achieve an efficient cleavage). Furthermore, it should be apparent that the reduction of pH (below 7) accelerates cleavage of the protecting groups.

In some embodiments, cleavage is accelerated at a pH value of less than 7. In some embodiments, the protecting group is not substantially cleaved at pH value above 7. In some embodiments, dissociation is deprotecting the plurality of amine groups. It will be understood by a skilled artisan that the protecting group protects the plurality of amine groups at neutral and basic pH, but at a pH of less than 7 the plurality of amine groups become deprotected. The deprotection is due to dissociation of the PG from the amines. In some embodiments, the dissociation is induced by cleavage of the PG. In some embodiments, dissociation produces an unmasked conjugate. In some embodiments, an unmasked conjugate is characterized by a positive zeta potential. In some embodiments, an unmasked conjugate comprises a positive zeta potential.

In some embodiments, the cell penetrating moiety of the invention is a charge masked moiety. In some embodiments, the protecting group is the masking. In some embodiments, the cell penetrating moiety of the invention is a charge masked moiety comprising an alkyl amine, a cationic polymer, including any a derivative or any a combination thereof, wherein the derivative comprises an alkyl amine and/or a cationic polymer bound to an amine protecting group. In some embodiments, the charge masked moiety comprises a cationic polymer bound to the protecting group of the invention (PG) wherein the PG is an amine protecting group capable of undergoing cleavage at a pH value of less than 7. In some embodiments, the charge masked moiety comprises an alkyl amine protected by the PG, and wherein the protected amine is capable of undergoing deprotection at a pH value of less than 7.0, less than 6.9, less than 6.8, less than 6.7, less than 6.5, less than 6.3, less than 6.0, less than 5.5, less than 5, less than 3, including any range between. In some embodiments, the protected amine comprises an amine salt (e.g., deprotonated amine) covalently bound to the PG.

In some embodiments, the cationic polymer (e.g., unprotected cationic polymer) comprises a plurality of amine groups. In some embodiments, the cationic polymer comprises a primary amine group, a secondary amine group, a tertiary amine group, or any combination thereof. In some embodiments, the cationic polymer is capable of undergoing ionization (positive ionization) within a solution having a pH value below the pKa value of the amine group of the cationic polymer. In some embodiments, the cationic polymer is capable of undergoing protonation within a solution having a pH value below the pKa value of the amine group of the cationic polymer (e.g., at a pH of less than 9, or less than 8).

In some embodiments, at least 50%, at least 70%, at least 80%, at least 90%, at least 95%, at least 99% by weight of the cationic polymer (e.g., unprotected cationic polymer) is positively charged (or protonated) within a solution having a pH value below the pKa value of the amine group of the cationic polymer, such as a pH of less than 9, less than 8, less than 7.5, less than 7, less than 6, less than 5, including any range between. In some embodiments, the cationic polymer (e.g., unprotected cationic polymer) undergoes multiple protonation within a solution, resulting in a plurality of positive surface charges, wherein the solution is as described herein.

In some embodiments, the cationic polymer comprises a polyamine. In some embodiments, the cationic polymer comprises polyethyleneimine (PEI). In some embodiments, the polyamine comprises primary amines. In some embodiments, the polyamine comprises secondary amines. In some embodiments, the polyamine comprises tertiary amines. In some embodiments, the polyamine comprises primary, secondary and tertiary amines. Polyamines are well known in the art and include for example polyethyleneimine and polypropyleneimine to name but a few.

In some embodiments, the cationic polymer (e.g., unprotected cationic polymer) is or comprises polyethyleneimine (PEI).

In some embodiments, the PEI comprises a linear PEI. In some embodiments, the PEI comprises a branched PEI. In some embodiments, the PEI (e.g., branched or linear PEI) is characterized by a number average molar mass (Mn) of less than 5000 Da, less than 4000 Da, less than 3000 Da, less than 2000 Da, less than 1500 Da, less than 1000 Da, less than 800 Da, including any range between.

In some embodiments, the cell penetrating moiety of the invention is characterized by MW (e.g., an average molecular weight) of between 100 and 2000 Da, between 200 and 5000 Da, between 200 and 3000 Da, between 500 and 5000 Da, between 500 and 2000 Da, between 500 and 3000 Da, between 100 and 300 Da, between 300 and 400 Da, between 400 and 500 Da, between 500 and 600 Da, between 600 and 700 Da, between 700 and 1000 Da, including any range between.

In some embodiments, the PEI is characterized by Mn of between 100 and 2000 Da, between 200 and 5000 Da, between 200 and 3000 Da, between 500 and 5000 Da, between 500 and 2000 Da, between 500 and 3000 Da between 100 and 300 Da, between 300 and 400 Da, between 400 and 500 Da, between 500 and 600 Da, between 600 and 700 Da, between 700 and 1000 Da, including any range between. In some embodiments, the cell penetrating moiety of the invention comprises a branched PEI characterized by Mn of between 500 and 700 Da, or between 500 and 2000 Da.

In some embodiments, the cell penetrating moiety of the invention comprises a plurality of PEIs, wherein the plurality of PEIs comprises between 3 and 10, between 4 and 10, between 2 and 10, between 4 and 20, between 4 and 50, between 4 and 100, between 2 and 100, between 2 and 50, between 2 and 20, between 4 and 8, between 6 and 10, between 6 and 9, between 6 and 8 PEI molecules covalently bound to a single protein carrier of the invention (e.g. HSA), including any range between.

In some embodiments, the cell penetrating moiety of the invention is a charge masked moiety comprising at least one protected amine. In some embodiments, the amine groups (e.g., primary and/or secondary amines) of the cell penetrating moiety (e.g. PEI) are substantially protected (e.g., at least about 40%, at least about 50%, between about 40 and about 95%, between about 40 and about 100%, between about 40 and about 70%, between about 40 and about 80%, between about 40 and about 90%, between about 40 and about 95%, or between about 40 and about 99% of amine groups are covalently bound to the protecting group), wherein the protecting group is represented by Formula 2 (optionally wherein the protecting group is derived from citraconic anhydride). In some embodiments, the cell penetrating moiety of the invention is covalently bound to a plurality of PGs, wherein each of the plurality of PGs has the same chemical structure. In some embodiments, the cell penetrating moiety of the invention is covalently bound to a plurality of PGs, wherein the plurality of PGs are or comprises chemically distinct PG species.

In some embodiments, the charge masked moiety comprises the payload of the invention bound to the PG. In some embodiments, the payload is bound to one or more PGs.

In some embodiments, the payload is bound to a plurality of PGs, wherein the PGs are chemically identical or chemically distinct species. In some embodiments, each payload molecule within the conjugate of the invention is covalently bound to one or more PGs, such as 1, 2, 3, 4, 5, 6, 7, 8 between 2 and 10, between 2 and 5, between 1 and 10, between 1 and 5, between 1 and 8, between 2 and 8 PGs, including any range between.

In some embodiments, the charge masked moiety is substantially devoid of protonation and/or positive charge (e.g., in an aqueous solution) at a pH ranging between 7 and 10. In some embodiments, the charge masked moiety and/or the protected plurality of amine groups is substantially uncharged or negatively charged (e.g., in an aqueous) solution at a pH ranging between 7 and 10, and is positively charged at a pH ranging below 7, below 6.8, below 6.5, below 6, including any range between. In some embodiments, the plurality of amine groups of the cell penetrating moiety are substantially uncharged or negatively charged (e.g., in an aqueous solution) at a pH ranging between 7 and 10 and are positively charged at a pH ranging below 7, below 6.8, below 6.5, below 6, including any range between. In some embodiments, the charge masked moiety is substantially devoid of amines (e.g., primary and/or secondary amines) capable of undergoing protonation at a pH ranging between 7 and 10. In some embodiments, the charge masked moiety has substantially reduced capability undergoing protonation at a pH ranging between 7 and 10, compared to unmasked cell penetrating moiety (devoid of PG).

In some embodiments, the charge masked moiety of the invention comprises one or more protected PEI (wherein at least a portion of the amines of PEI is bound to the PG). In some embodiments, the protected PEI comprises one or more amines (e.g. deprotonated amines) of PEI bound to the protecting group of the invention. In some embodiments, the cell penetrating moiety of the invention comprises a linear or branched PEI, at least 1%, at least 5%, wherein at least 10%, at least 20%, at least 30%, at least 40%, at least 50%, at least 70%, at least 80%, at least 90%, at least 95%, or between 50 and 99%, between 50 and 97%, between 50 and 95%, between 50 and 90%, between 50 and 85%, between 50 and 80%, of the amine groups (e.g., primary amines and/or secondary amines) are covalently bound to a protecting group of the invention, including any range between.

In some embodiments, any of: the charge masked moiety, the protein conjugate of the invention, and the protein carrier of the invention is negatively charged at a pH between 7 and 10, between 7.2 and 10, or at between 7.5 and 10, including any range between, or at a pH greater than 7. In some embodiments, any of: the charge masked moiety, the protein conjugate of the invention, and the protein carrier of the invention is negatively charged at a pH of about 7.4.

In some embodiments, any of: the cell penetrating moiety, the protein conjugate of the invention, the payload, and the protein carrier of the invention is characterized by a zeta potential of less than −0.1, less than −0.5, less than −1, less than −5, less than −10, less than −12, less than −14, less than −20 mV including any range between. In some embodiments, a negative zeta potential is a zeta potential below 0. In some embodiments, a negative zeta potential is a zeta potential −1 mV or below. In some embodiments, a negative zeta potential is a zeta potential −2 mV or below. In some embodiments, a negative zeta potential is a zeta potential −3 mV or below. In some embodiments, a negative zeta potential is a zeta potential −4 mV or below. In some embodiments, a negative zeta potential is a zeta potential −5 mV or below.

In some embodiments, a molar ratio between the PG and the cationic polymer (e.g., PEI) within the charge masked moiety is between 100,000:1, and 0.8:1, 50,000:1 and 1:1, 25,000:1 and 1:1, 10,000:1 and 1:1, 8,000:1 and 1:1, 5,000:1 and 1:1, 3,000:1 and 1:1, 2,000:1 and 1:1, 1,000:1 and 1:1, 900:1 and 1:1, 800:1, and 1:1, 700:1 and 1:1, 600:1 and 1:1, 500:1 and 1:1, 400:1 and 1:1, 300:1 and 0.8:1, 250:1 and 1:1, 200:1 and 0.8:1, 150:1 and 1:1, 125:1 and 1:1, 100:1 and 0.8:1, between 100:1 and 80:1, between 80:1 and 50:1, between 50:1 and 30:1, between 30:1 and 10:1, between 10:1 and 5:1, between 10:1 and 0.8:1, between 5:1 and 1:1, between 1:1 and 0.8:1, including any range between.

In some embodiments, a molar ratio between the PG and the carrier (e.g., HSA) is between 100,000:1, and 1:1, 50,00:1 and 1:1, 25,000:1 and 1:1, 10,000:1 and 1:1, 8,000:1 and 1:1, 5,000:1 and 1:1, 3,000:1 and 1:1, 2,000:1 and 1:1, 1,000:1 and 1:1, 900:1 and 1:1, 800:1, and 1:1, 700:1 and 1:1, 600:1 and 1:1, 500:1 and 1:1, 400:1 and 1:1, 300:1 and 1:1, 250:1 and 1:1, 200:1 and 1:1, 150:1 and 1:1, 125:1 and 1:1, 100:1 and 1:1, between 100:1 and 80:1, between 80:1 and 50:1, between 50:1 and 30:1, between 30:1 and 10:1, between 10:1 and 5:1, between 5:1 and 1:1, including any range between.

In some embodiments, the protected amine of the charge masked moiety is substantially stable (devoid of deprotection) at a neutral and/or basic pH. In some embodiments, at least 50%, at least 60%, at least 70%, at least 80%, at least 90%, at least 95 (mole %) of the protected amines of the charge masked moiety, including any range between, remain stable at a neutral and/or basic pH for a time period as described hereinbelow.

In some embodiments, the protected amine is substantially stable at a pH ranging between 7.0 and 14, between 7.0 and 7.2, between 7.2 and 7.5, between 7.5 and 8, between 8 and 9, between 9 and 12, between 12 and 14, including any range between. In some embodiments, the protected amine is substantially stable at a pH of about 7.4. In some embodiments, the protected amine is substantially stable at a pH value ranging between 7.0 and 14, for at least 1 h, at least 2 h, at least 10 h, at least 24 h, at least 48 h, or at least 72 h, including any range between.

In some embodiments, the protected amine is capable of undergoing deprotection (or degradation via cleavage of the PG therefrom), to result in a deprotected amine (e.g., uncharged or positively charged protonated amine). In some embodiments, the protected amine substantially undergoes deprotection at a pH ranging between 0 and 6.9, between 0 and 6.8, between 6 and 6.8, between 5 and 6, between 0 and 3, between 3 and 5, between 5 and 6.8, including any range between. In some embodiments, the protected amine substantially undergoes deprotection at a pH of about 6.8. In some embodiments, the protected amine substantially undergoes deprotection in a cancer microenvironment. In some embodiments, a cancer microenvironment is a tumor microenvironment (TME). In some embodiments, at least 10%, at least 20%, at least 30%, at least 40%, at least 50%, at least 60%, at least 70%, at least 80%, at least 90%, at least 95 (mole %) of the protected amines undergo deprotection at a pH below 7 (e.g., between 0 and 6.9), including any range between.

In some embodiments, the protected amine substantially undergoes deprotection at a pH below 7 (e.g., between 0 and 6.9, between 0 and 6.8, between 6 and 6.8, between 5 and 6, between 0 and 3, between 3 and 5, between 5 and 6.8, including any range between) within a time period ranging between 1 second (s) and 1 hour (h), between 1 and 30 s, between 30 and 60 s, between 60 s and 2 minutes (m), between 2 and 10 m, between 1 m and 1 h, between 1 m and 24 h, between 1 m and 12 h, between 1 m and 8 h, between 1 m and 6 h, between 1 m and 4 h, between 1 m and 3 h, between 1 m and 2 h, between is and 24 h, between is and 12 h, between 1 s and 8 h, between 1 s and 6 h, between 1 s and 4 h, between 1 s and 3 h, between 1 s and 2 h, including any range between. In some embodiments, a pH below 7 is about 6.8.

### Protecting Group

In some embodiments, the protected amine comprises PG of the invention covalently bound to an amine. In some embodiments, the protected amine is obtained by reacting a PG precursor with an amine. In some embodiments, the PG precursor has a reactivity towards an amine (e.g., a primary amine, a secondary amine or both). In some embodiments, the PG precursor is or comprises a cyclic anhydride (e.g., 5-6 membered optionally unsaturated cyclic anhydride), optionally substituted (e.g., with R and R1, as described hereinbelow). In some embodiments, the PG precursor is capable of reacting with an amine so as to form a stable protected amine. In some embodiments, the protected amine (e.g., within the charge masked moiety) is stable under neutral and/or basic pH conditions. In some embodiments, the PG precursor is capable of reacting with an amine group (primary and/or secondary amine), thereby converting the amine group into a protected amine (such as an amide). In some embodiments, the PG precursor is a cyclic anhydride of Formula:

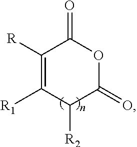

wherein n, R, R1 and R2 are as described hereinabove.

In some embodiments, the PG of the invention is covalently bound to an amine, wherein the amine is selected from: (i) an amine group of the payload, (ii) an amine group of the cell penetrating moiety, or both (i) and (ii).

In some embodiments, the PG (protecting group bound to an amine or a deprotonated amine) comprises one or more moieties (e.g., 1, 2, 3, or 4 moieties) having a negative charge at a pH above 5. In some embodiments, the PG is negatively charged at a pH between 4 and 8, between 4 and 5, between 5 and 6, between 6 and 7, between 7 and 8 or more, including any range between. In some embodiments, the PG is substantially negatively charged within a tissue or within a biological fluid of a subject, wherein the tissue and/or the biological fluid is characterized by a pH of between 4 and 8, or between 5 and 9, including nay range between.

In some embodiments, at least 50%, at least 60%, at least 70%, at least 80%, at least 90%, at least 95 (mole %) of the PGs are negatively charged at a pH above a pKa value of the moiety. In some embodiments, the moiety is or comprises carboxy or a salt thereof. In some embodiments, the PG comprises carboxy and/or a salt thereof, and wherein the PG negatively charged at a pH of between 4 and 8, or of between 5 and 8 or at a pH greater than 8.

In some embodiments, the PG is represented by Formula 1:

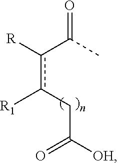

wherein n is an integer ranging from 0 to 5; ----represents an attachment point to the amine (e.g., a nitrogen atom of the protected amine), and [symbol] represents a single bond or a double bond; R and R1 each independently represent one or more substituent selected from H, optionally substituted alkyl (e.g., C1-C10 alkyl, or C1-C5 alkyl), halo, optionally substituted cycloalkyl, optionally substituted aryl or heteroaryl, and carboxyalkyl (e.g., C1-C10 carboxyalkyl, or C1-C5 carboxyalkyl), or any combination thereof; or R and R1 are bound together so as to form a cyclic ring.

In some embodiments, R and R1 each independently represent one or more substituents selected from H, C₁-C₁₀ alkyl, C₁-C₁₀ alkenyl, —NO₂, —CN, —OH, —NH₂, carbonyl, —CONH₂, —CONR′₂, —CNNR′₂, —CSNR′₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, —NH(C₁-C₆ alkyl), —N(C₁-C₁₀ alkyl)₂, C₁-C₁₀ alkoxy, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —OC(═O)OR′, —OC(═O)NR′, —OC(═S)OR′, —OC(═S)NR′, a heteroatom, an optionally substituted cycloalkyl, an optionally substituted heterocyclyl, or any combination thereof; wherein each R′ independently comprises hydrogen, optionally substituted C1-C₆ alkyl, optionally substituted cycloalkyl, optionally substituted heterocyclyl, optionally substituted phenyl, optionally substituted benzyl or a combination thereof.

In some embodiments, n is between 0 and 5, between 0 and 1, between 1 and 5, between 1 and 3, between 3 and 5, including any range between. In some embodiments, each R and/or R1 represents one or more substituents. In some embodiments, R and R1 represent the same or different substituent(s). In some embodiments, carboxyalkyl comprises -alkyl-COOH. In some embodiments, carboxyalkyl comprises —(C1-C10)alkyl-COOH, or —(C1-C5)alkyl-COOH, including any range between, wherein alkyl is optionally substituted.

In some embodiments, the PG is represented by Formula 2:

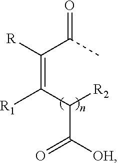

wherein n, R and R1 are as described herein, and wherein each R2 independently comprises one or more substituents (e.g. 1 or 2 substituents) each independently selected from H, C₁-C₁₀ alkyl, C₁-C₁₀ alkenyl, —NO₂, —CN, —OH, —NH₂, carbonyl, —CONH₂, —CONR′₂, —CNNR′₂, —CSNR′₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, —NH(C₁-C₆ alkyl), —N(C₁-C₁₀ alkyl)₂, C₁-C₁₀ alkoxy, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —OC(═O)OR′, —OC(═O)NR′, —OC(═S)OR′, —OC(═S)NR′, a heteroatom, an optionally substituted cycloalkyl, an optionally substituted heterocyclyl, or any combination thereof; wherein each R′ independently comprises hydrogen, optionally substituted C1-C₆ alkyl, optionally substituted cycloalkyl, optionally substituted heterocyclyl, optionally substituted phenyl, optionally substituted benzyl or a combination thereof.

In some embodiments, one of R and R1 is H, and another one of R and R1 comprises an alkyl or a carboxyalkyl. In some embodiments, each R and R1 independently comprises a C₁-C₁₀ alkyl (a branched or a linear), or a carboxyalkyl.

In some embodiments, the PG is represented by Formula 2, wherein n is 0; R is selected from CH₂COOH, methyl, and ethyl; and R1 is selected from H, and CH₂CH₂COOH.

In some embodiments, the PG is represented by Formula 2A:

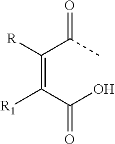

wherein R and R1 are selected from H and methyl, and wherein R or R1 is methyl.

In some embodiments, the PG is represented by any of the Formulae disclosed herein, including any salt (e.g., carboxylate salt), any derivative, any tautomer, any isotope, or any structural isomer thereof. In some embodiments, the PG is a protective group disclosed hereinbelow. In some embodiments, the PG is a maleic anhydride derivative. In some embodiments, the PG is derived from citraconic anhydride. In some embodiments, the PG is maleic anhydride. In some embodiments, the PG is derived from cis aconitic anhydride. In some embodiments, the PG is derived from dimethyl maleic anhydride.

As used herein, the term “derived from” encompasses a molecule obtained via nucleophilic substitution of the PG precursor (e.g., a cyclic anhydride) with an amine group. In some embodiments, the PG is derived from citraconic anhydride, wherein the protecting group is represented by Formula 2A, and wherein R or R1 is methyl. In some embodiments, the PG is or comprises citraconic anhydride. In some embodiments, the PG is derived from citraconic anhydride and has the form of Formula 2A.

In some embodiments, the protein carrier comprises a plurality of PEI molecules covalently bound thereto. In some embodiments, the protein carrier comprises between 3 and 10, or between 4 and 10, between 2 and 100, between 3 and 100, between 3 and 90, between 4 and 100, between 4 and 90, between 4 and 10, between 4 and 40, between, 4 and 20, between 20 and 100, between 20 and 40, between 40 and 60, between 60 and 100, between 6 and 100, between 6 and 20 between 6 and 40, between 6 and 50, between 4 and 15, between 3 and 15, between 3 and 10, between 3 and 8, between 4 and 8, between 6 and 10 PEI molecules covalently bound thereto, including any range between.

In some embodiments, the protein carrier comprises between 3 and 10 PEI molecules, between 3 and 5 PEI molecules, between 5 and 8 PEI molecules, between 8 and 10 PEI molecules, between 10 and 15 PEI molecules, between 15 and 20 PEI molecules covalently bound thereto, including any range between.

In some embodiments, the protein carrier comprises a size of at least 20, 25, 30, 35, 40, 45, 50, 55, 60, 65, 70, 75, or 80 KDa. Each possibility represents a separate embodiment of the invention. In some embodiments, the protein carrier comprises a size of at least 50 KDa. In some embodiments, the protein carrier comprises a size of at least 60 KDa. In some embodiments, the protein carrier comprises a size of at least 65 KDa.

In some embodiments, the protein carrier is a blood endogenous protein. In some embodiments, the protein is naturally found in blood. In some embodiments, blood is plasma. In some embodiments, the blood is mammalian blood. In some embodiments, the mammal is humans. In some embodiments, the blood endogenous protein is an albumin. In some embodiments, the blood endogenous protein is a globulin. In some embodiments, the blood endogenous protein is a fibrinogen. In some embodiments, the globulin is an immunoglobulin (Ig). In some embodiments, the Ig is IgG. In some embodiments, the Ig is IgA. In some embodiments, the Ig is IgM. In some embodiments, the blood endogenous protein is selected from HSA, fibrinogen, and IgG. In some embodiments, the blood endogenous protein is not a clotting protein. Blood endogenous proteins are well known in the art and include, for example, Prealbumin (transthyretin), Alpha 1 antitrypsin, Alpha-i-acid glycoprotein, Alpha-i-fetoprotein, alpha2-macroglobulin, Gamma globulins, Beta-2 microglobulin, Haptoglobin, Ceruloplasmin, Complement component 3, Complement component 4, C-reactive protein (CRP), Lipoproteins, (chylomicrons, VLDL, LDL, HDL), Transferrin, Prothrombin, and maltose binding protein (MBP) to name but a few. In some embodiments, the protein carrier is selected from human serum albumin (HSA), fibrinogen, IgG, a fluorescent protein (GFP) and a designed ankyrin repeat protein (DARPin). In some embodiments, the fluorescent protein is selected from green (GFP), red (RFP), blue (BFP) and yellow (YFP). In some embodiments, the fluorescent protein is GFP. In some embodiments, the protein carrier is HSA. In some embodiments, the protein carrier is fibrinogen. In some embodiments, the protein carrier is IgG.

In some embodiments, the protein carrier is or comprises HSA. In some embodiments, the protein carrier comprises a PEI modified HSA. In some embodiments, the protein conjugate comprises a payload bound to the protein carrier via a linker, wherein the linker is as described herein, and wherein the protein carrier is or comprises HSA covalently bound to (or modified by) between 3 and 20, between 3.5 and 20, between 3 and 10, between 3.5 and 10, between 3 and 8, between 3.5 and 8, between 3 and 5, between 5 and 8, between 8 and 10, between 10 and 15, between 3 and 10, between 3 and 15, between 4 and 20, between 4 and 10, between 3.5 and 6, between 3.5 and 15, between 3.5 and 8, between 3.5 and 12, between 3 and 12, between 3 and 17, between 3.5 and 17, between 3.5 and 15, between 4 and 8, between 6 and 10 PEI molecules, including any range between. In some embodiments, the number of PEI molecules described herein represents an average value. In some embodiments, the PEI molecules are characterized by an average MW of between 100 and 2000 Da, between 200 and 5000 Da, between 200 and 3000 Da, between 500 and 5000 Da, between 500 and 2000 Da, between 500 and 3000 Da, between 100 and 300 Da, between 300 and 400 Da, between 400 and 500 Da, between 500 and 600 Da, between 600 and 700 Da, between 700 and 1000 Da, including any range between. In some embodiments, the protein conjugate comprises PEI and is masked by between 15-50, 15-45, 15-35, 15-30, 15-25, 20-50, 20-45, 20-40, 20-35, 20-25, 25-50, 25-45, 25-40, 25-35, 25-30, 30-50, 30-45, 30-40, 30-35, 35-50, 35-45, 35-40, 40-50, 40-45, or 45-50 molecules of PG, including any range between.

In some embodiments, the protein carrier is covalently bound to at least 2 molecules of PEI. In some embodiments, the protein carrier is covalently bound to at least 8 molecules of PEI. In some embodiments, the biological payload is covalently bound to 1 molecule of PEI. In some embodiments, the protein conjugate is covalently bound to at least 3 molecules of PEI. In some embodiments, the protein conjugate is covalently bound to at least 8 molecules of PEI. In some embodiments, the protein conjugate is covalently bound to at least 98 molecules of PEI.

In some embodiments, the conjugate comprises a payload. As used herein, the term “payload” refers to any molecule to be delivered into the cytoplasm of a target cell. In some embodiments, the payload binds an intracellular target. In some embodiments, the payload interacts with an intracellular target. In some embodiments, the payload modulates an intracellular target. In some embodiments, intracellular is cytoplasmic. In some embodiments, the payload is devoid of a disulfide bond that when cleaved diminishes interaction with the intracellular target. In some embodiments, the payload is devoid of a disulfide bond. In some embodiments, the payload is a molecule. In some embodiments, the payload is a biological payload. In some embodiments, the payload is a biological molecule. In some embodiments, the payload is organic. In some embodiments, the payload is a therapeutic molecule. In some embodiments, the payload is a detectable molecule. In some embodiments, the payload is a molecule capable of binding to a target. In some embodiments, the payload is a biologic. In some embodiments, the payload is a drug. In some embodiments, the payload is a protein or peptide. In some embodiments, the peptide or protein is an isolated protein or peptide. In some embodiments, the peptide or protein is a peptide or protein moiety. It will be understood that the protein need not be a complete protein but may be a portion or fragment of a protein. In some embodiments, the payload comprises or consists of amino acids. In some embodiments, the payload is a single amino acid chain. In some embodiments, the payload is a plurality of amino acid chains. In some embodiments, the payload is a bioactive molecule. In some embodiments, a bioactive molecule is a bioactive agent.

In some embodiments, the payload is a nucleic acid molecule. In some embodiments, the payload is DNA. In some embodiments, the payload is RNA. In some embodiments, the nucleic acid molecule is an oligonucleotide. In some embodiments, the payload is an aptamer. In some embodiments, the payload is a primer. In some embodiments, the payload is an antisense oligonucleotide. In some embodiments, the payload is a regulatory RNA. In some embodiments, the payload is plasmid. In some embodiments, the payload is an expression vector. In some embodiments, the vector is configured to expresses in a target cell. In some embodiments, the payload is gene therapy. In some embodiments, the nucleic acid molecule comprises an open reading frame. In some embodiments, the open reading frame encodes a therapeutic protein. Methods of conjugating nucleic acid molecules to chemical and amino acid linkers are well known in the art and any such method may be employed. In some embodiments, the nucleic acid molecule comprises a nuclear localization signal (NLS). In some embodiments, the payload is selected from a protein and a nucleic acid molecule.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

As used herein, the term “oligonucleotide” refers to a short (e.g., no more than 100 bases), chemically synthesized single-stranded DNA or RNA molecule. In some embodiments, oligonucleotides are attached to the 5′ or 3′ end of a nucleic acid molecule, such as by means of ligation reaction.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art and herein its delivery may be performed by a method of the invention or using a composition of the invention. In some embodiments, the gene is in an expression vector such as plasmid or viral vector. The vector may be a viral vector. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene or open reading frame is operably linked to a promoter or other regulatory element. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell by a method of the invention). In some embodiments, the regulatory element or promoter is active in a target cell.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

The term “bioactive” refers to a molecule or agent that exerts an effect on a cell or tissue. Representative examples of types of bioactive agents include therapeutics, vitamins, electrolytes, amino acids, peptides, polypeptides, proteins, enzymes, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, neurotransmitters, prostaglandins, immunoglobulins, cytokines, and antigens. Various combinations of these molecules can be used. Examples of cytokines include macrophage derived chemokines, macrophage inflammatory proteins, interleukins, tumor necrosis factors. Examples of proteins include fibrous proteins (e.g., collagen, elastin) and adhesion proteins (e.g., actin, fibrin, fibrinogen, fibronectin, vitronectin, laminin, cadherins, selectins, intracellular adhesion molecules, and integrins). In various cases, the bioactive agent may be selected from fibronectin, laminin, thrombospondin, tenascin C, leptin, leukemia inhibitory factors, RGD peptides, anti-TNFs, endostatin, angiostatin, thrombospondin, osteogenic protein-1, bone morphogenic proteins, osteonectin, somatomedin-like peptide, osteocalcin, interferons, and interleukins. In some embodiments, the bioactive agent includes a growth factor, differentiation factor, or a combination thereof.

As used herein, the term “isolated peptide” refers to a peptide that is essentially free from contaminating cellular components, such as carbohydrate, lipid, or other proteinaceous impurities associated with the peptide in nature. Typically, a preparation of isolated peptide contains the peptide in a highly purified form, i.e., at least about 80% pure, at least about 90% pure, at least about 95% pure, greater than 95% pure, or greater than 99% pure.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the payload binds a cytoplasmic target. In some embodiments, the payload is specific to the cytoplasmic target. In some embodiments, specific comprises not significantly binding to any other target. In some embodiments, the payload is a binding molecule. In some embodiments, the payload hybridizes to its target. In some embodiments, the payload is complementary to its target. In some embodiments, the payload comprises complementarity determining regions (CDRs) that bind the target. In some embodiments, the payload is an antibody or antigen binding fragment thereof. The structure of antibodies is well known and though a skilled artisan may not know to what target an antibody binds merely by its CDR sequences, the general structure of an antibody and its antigen binding region can be recognized by a skilled artisan.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and scFv-scFv-Fc fusions.

Immunoglobulin molecules can be of any type (e.g., IgG, IgE, IgM, IgD, IgA and IgY), class (e.g., IgGl, IgG2, IgG3, IgG4, IgA1 and IgA2) or subclass.

In some embodiments, the antibody is a single chain antibody (ScFv). In some embodiments, the antibody is a single domain antibody. In some embodiments, the antibody is a camelid antibody. In some embodiments, the antibody is a shark antibody. In some embodiments, the antibody is a VHH. In some embodiments, the antibody comprises a heavy chain and a light chain. In some embodiments, the antibody is a heavy chain only antibody. In some embodiments, the antibody is an antibody mimetic. In some embodiments, the binding molecule or antibody mimetic is a DARPin. Regardless of the CDRs present antibodies, antibody fragments, ScFvs, nanobodies, VHHs, single domain antibodies, DARPins and the like can be structurally recognized by their non-variable regions. Thus, without being limited to a specific target a composition of the invention can be known to comprise these molecules as payload.

In some embodiments, the payload comprises a C-terminal cysteine amino acid. In some embodiments, the payload comprises a cysteine amino acid proximal to the C-terminus of the payload. In some embodiments, proximal is within 10, 9, 8, 7, 6, 5, 4, 3, 2, or 1 amino acid of the terminus. Each possibility represents a separate embodiment of the invention.

In some embodiments, the payload comprises more than one molecule. In some embodiments, the payload comprises more than one bioactive molecule. In some embodiments, the payload is bispecific. As used herein, the term “bispecific” refers to having a function against two different targets. In some embodiments, the payload comprises 1, 2, 3, 4, 5, 6, 7, 8, 9 or 10 molecules. Each possibility represents a separate embodiment of the invention. In some embodiments, the payload comprises at least 2 molecules against two intracellular targets. In some embodiments, at least 2 is 2. In some embodiments, the two targets are the same targets. In some embodiments, the two targets are different targets. In some embodiments, the payload comprises at least two VHHs. In some embodiments, the at least two VHHs are specific to different intracellular proteins.

In some embodiments, the at least two molecules are separated by a linker. In some embodiments, the linker is a cleavable linker. In some embodiments, the linker is cleavable in the cytoplasm. In some embodiments, the linker is flexible. In some embodiments, cleavage of the linker allows each molecule to reach its target. In some embodiments, the linker is not cleavable. In some embodiments, the two molecules bring two target intracellular proteins together.

In some embodiments, the protein conjugate comprises a payload bound to the protein carrier via a linker. In some embodiments, the protein conjugate is not a fusion protein. It will be understood by the skilled artisan that the payload and carrier are conjugated to each other by a separate linker. The linker is not part of the carrier and is also not a part of the payload, but rather is attached (conjugated) to each one and thereby links them.

In some embodiments, the linker of the invention is substantially stable within a biological fluid (e.g., human blood, plasma or serum) for at least 2, at least 10, at least 24, at least 48 hours, including any range between. Each possibility represents a separate embodiment of the invention. In some embodiments, the linker of the invention is substantially stable within blood. In some embodiments, blood is human blood. In some embodiments, the blood is murine blood. In some embodiments, the blood is rodent blood. In some embodiments, the rodent is a rat. In some embodiments, the rodent is a mouse. In some embodiments, the linker of the invention is substantially stable within human blood for at least 1, 2, 3, 4, 5, 6, 7, 8, 9, 10, 11, 12, 18, 24, 36, 48 or 72 hours. Each possibility represents a separate embodiment of the invention.

In some embodiments, the linker of the invention and/or the charge masked moiety is labile under exposure to cytoplasmic conditions. In some embodiments, the linker of the invention is cleavable under exposure to cytoplasmic conditions, so as to release the biological payload to a cytosol.

In some embodiments, the charge masked moiety undergoes cleavage or deprotection under exposure to conditions comprising a pH between 6 and 7, so as to result in a protein carrier (e.g., HSA) comprising deprotected cell penetrating moieties (e.g., deprotected PEI molecules). In some embodiments, a pH between 6 and 7 is about 6.8.

In some embodiments, the protein carrier comprising deprotected cell penetrating moieties (e.g., deprotected PEI molecules) is characterized by a positive zeta potential value of of at least 5 mV, at least 6, at least 7, at least 8, at least 8.5, at least 9, at least 9.5, at least 10, at least 12 mV, or between 8 and 40, between 8.5 and 40, between 8 and 20, between 8.5 and 20, between 10 and 40, between 10 and 20, between 10 and 30 mV, between 5 and 50 mV, between 6 and 50 mV, between 7 and 50 mV, between 8 and 50 mV, between 8 and 40 mV, between 8 and 30 mV, between 8 and 20 mV, between 10 and 50 mV, between 10 and 40 mV, between 10 and 30 mV, between 10 and 20 mV, between 20 and 50 mV, between 30 and 50 mV, including any range between. A skilled artisan will appreciate that the exact zeta potential value of the conjugate may vary, depending on the MW and/or size of the protein carrier, of the payload or both.

In some embodiments, the protein conjugate comprising deprotected cell penetrating moieties (e.g., deprotected PEI molecules) is characterized by a positive zeta potential value of at least 6, at least 7, at least 8, at least 8.5, at least 9, at least 9.5, at least 10, at least 12 mV, or between 8 and 40, at least 8 mV, between 8.5 and 40, between 8 and 20, between 8.5 and 20, between 10 and 40, between 10 and 20, between 10 and 30 mV, including any range between. Each possibility represents a separate embodiment of the invention.

Zeta potential may be measured by any method known in the art. Herein the following protocol is used and can be considered the standard for determining if a molecule comprises a zeta potential in the herein recited range. Zeta potential measurements were preformed using Zeta Sizer Ultra (Malvern Instruments). Samples' buffers were exchanged to 1 mM NaCl at 1 mg/mL protein concentration. 20 μL from each sample was loaded in zeta cells (DTS1070), five repeats for each sample were measured and the mean zeta potential in mV was obtained for each repeat. The average for the five measurements is reported with the standard deviation. The measurements were performed under the following conditions: temperature 25° C.; run numbers for each repeat 10-40; equilibration time: 60 seconds, without pause after sub runs; 60 seconds pause between repeats; voltage was selected automatic and monomodal analysis method was used in the data processing. In some embodiments, the zeta potential is measured in about 1 mM salt. In some embodiments, the salt is NaCl. In some embodiments, the zeta potential is measured at a protein concentration of about 1 mg/mL.

In some embodiments, the protein conjugate of the invention comprises the protein carrier covalently bound to the payload via a linker. In some embodiments, the payload is covalently bonded to the linker. In some embodiments, the carrier is covalently bonded to the linker. In some embodiments, the covalent bond is not a peptide bond. In some embodiments, the at least one of the bond between the linker and the payload and the bond between the linker and the carrier is not a peptide bond. In some embodiments, the carrier, linker and payload are not comprised in a single amino acid chain. In some embodiments, the protein conjugate of the invention comprises the protein carrier covalently bound to the payload via a linker, wherein the linker is a synthetic linker comprising at least one cleavable bond. In some embodiments, the protein conjugate of the invention comprises the protein carrier covalently bound to the payload via a linker, wherein the linker is a synthetic linker devoid of a cleavable bond. In some embodiments, the carrier and payload are not from the same protein. In some embodiments, the linker and the carrier are not from the same protein. In some embodiments, the linker is a peptide linker and comprises a sequence not present in the amino acid sequence of the protein from which the carrier is based. In some embodiments, the linker is a peptide linker and comprises a sequence not present in the amino acid sequence of the protein from which the payload is based. In some embodiments, the linker and the payload are not from the same protein. In some embodiment, the payload is not a naturally occurring molecule. In some embodiments, the payload is manmade. In some embodiments, the linker is not naturally occurring. In some embodiments, the linker is manmade. In some embodiments, the carrier is a naturally occurring protein or fragment thereof.

In some embodiments, the linker is a protein linker. In some embodiments, the linker is a peptide linker. In some embodiments, the linker is an amino acid linker. In some embodiments, the linker is a rigid linker. In some embodiments, the linker is a flexible linker. In some embodiments, the rigid linker is an alpha-helical peptide. In some embodiments, the linker is a flexible linker. In some embodiments, the flexible linker is a GGGGS linker. In some embodiments, the linker comprises a C-terminal cysteine amino acid. In some embodiments, the linker comprises a cysteine amino acid proximal to the C-terminus of the linker. In some embodiments, proximal is within 10, 9, 8, 7, 6, 5, 4, 3, 2, or 1 amino acid of the terminus. Each possibility represents a separate embodiment of the invention. In some embodiments, the linker comprises an N-terminal cysteine and the payload comprises a C-terminal cysteine. It will be understood by a skilled artisan that the side chain of the cysteine comprises a sulfur atom that can be used to generate a cleavable disulfide bond. For example, the cysteine can form a disulfide bond with cysteine 34 of HSA.

In some embodiments, the alpha-helical peptide comprises or consists of AAASAEAAAKEAAAKEAAAKAAAGSG (SEQ ID NO: 6). In some embodiments, the alpha-helical peptide comprises or consists of AAASAEAAAKEAAAKEAAAKAAAGSGLC (SEQ ID NO: 10). In some embodiments, the alpha-helical peptide comprises or consists of AAASAEAAAKEAAAKEAAAKAAAGSGL (SEQ ID NO: 14). In some embodiments, the flexible linker is a GGGGS linker. In some embodiments, the flexible linker comprises between 1-5 GGGGS repeats. In some embodiments, 1-5 is 1-3. In some embodiments, 1-5 is 1. In some embodiments, 1-5 is 2. In some embodiments, 1-5 is 3. In some embodiments, the linker comprises or consists of GGGGSGGGGSGGGGLC (SEQ ID NO: 4). In some embodiments, the linker comprises or consists of GGGGSGGGGSGGGLGC (SEQ ID NO: 5). In some embodiments, the linker comprises or consists of GGGGSGGGGSGGGLG (SEQ ID NO: 7). In some embodiments, the linker comprises or consists of GGGGSGGGGSGGGGSC (SEQ ID NO: 8). In some embodiments, the linker comprises or consists of GGGGSGGGGSGGGGS (SEQ ID NO: 12). In some embodiments, the linker comprises or consists of GGGGSC (SEQ ID NO: 9). In some embodiments, the linker comprises or consists of GGGGS (SEQ ID NO: 13). In some embodiments, the linker comprises or consists of GGGLGC (SEQ ID NO: 11). In some embodiments, the linker comprises or consists of GGGLG (SEQ ID NO: 15). In some embodiments, the linker comprises or consists of an amino acid sequence selected from SEQ ID NO: 4-15.

In some embodiments, the protein conjugate of the invention is substantially devoid of a biocleavable bond (e.g., a bond cleavable under exposure of the protein conjugate to cytoplasmic conditions). In some embodiments, the linker is substantially devoid of a biocleavable bond. In some embodiments, the linker is attached to the protein carrier and/or to the payload via a non-biocleavable bond (e.g., an amide bond, a click reaction product, a thioether bond, etc.).

In some embodiments, the linker of the invention comprises a bio cleavable bond. In some embodiments, the bio cleavable bond is substantially stable within a biological fluid (e.g., human blood, plasma or serum) for at least 2 h, at least 10 h, at least 24 h, at least 48 h, including any range between. In some embodiments, cleavable is cleavable in the cytoplasm. In some embodiments, cleavable is not cleavable in serum or blood. In some embodiments, not cleavable is not substantially cleavable. In some embodiment, bio cleavable in the cytoplasm is significantly more cleaved in the cytoplasm than in blood.

In some embodiments, the bio cleavable bond is cleavable under exposure to cytoplasmic conditions. In some embodiments, the bio cleavable bond is reducible under exposure to cytoplasmic conditions (e.g., intracellular compartment, comprising inter alia acidic pH conditions and/or reducing agents such as glutathione).

Bio cleavable bonds are well-known in the art and refer to bonds which are selectively cleaved after entering the cell (intracellular cleavage). The preferred linkages for release of drugs within the cell are cleavable in acidic conditions like those found in lysosomes. One example is a disulfide bond. It is postulated, that the disulfide bond is cleaved upon entering the cell by glutathione. In some embodiments, a bio cleavable bond is a bond cleaved intracellularly. In some embodiments, cleaved intracellularly is cleaved inside a cell. In some embodiments, inside a cell is in a cytoplasm of a cell. In some embodiments, inside a cell is in a vesicle of a cell. In some embodiments, the vesicle is an endosome. In some embodiments, the vesicle is a lysosome. In some embodiments, the vesicle is a vesicle of the Golgi. In some embodiments, the cleavage is selective cleavage.

In some embodiments, selective is as compared to cleavage extracellularly. In some embodiments, extracellularly is outside the cell. In some embodiments, outside the cell is in a biological fluid.

In some embodiments, the bio cleavable bond is or comprises a disulfide bond. In some embodiments, the bio cleavable bond comprises a plurality of disulfide bonds. In some embodiments, the bio cleavable bond is sterically hindered. In some embodiments, the bio cleavable bond is or comprises a sterically hindered disulfide bond.

In some embodiments, a biological fluid is a bodily fluid. In some embodiments, the biological fluid is selected from at least one of: blood, serum, plasma, gastric fluid, intestinal fluid, saliva, bile, tumor fluid, breast milk, urine, interstitial fluid, cerebral spinal fluid and stool. In some embodiments, the biological fluid is blood. In some embodiments, the biological fluid is serum. In some embodiments, the biological fluid is plasma.

In some embodiments, the sterically hindered disulfide bond comprises a side group or a bulky moiety adjacent thereto. In some embodiments, the side group or the bulky moiety is located in close proximity to at least one sulfur atom of the disulfide bond. In some embodiments, adjacent or in close proximity comprises a distance ranging between 0 and 10, between 0 and 2, between 2 and 5, between 5 and 10 atomic bonds, including any range between. In some embodiments, the term “atomic bond” as used herein, refers to carbon-carbon (C—C) bond length, e.g., a single C—C bond length.

In some embodiments, the sterically hindered disulfide bond comprises a side group or a bulky moiety adjacent thereto (e.g., positioned at a distance ranging from 1 to 15 Å, from 1 to 3 Å, from 3 to 5 Å, from 5 to 10 Å, from 10 to 15 Å from a sulfur atom of the disulfide bond, including any range between).

In some embodiments, the side group or a bulky moiety comprises an alkyl (e.g., a primary, a secondary or a tertiary C1-C10 alkyl, optionally comprising an unsaturated bond and/or a substituent), an aromatic ring, or an amino acid comprising a sterically hindered side chain (e.g., leucine, valine, isoleucine, phenylalanine, histidine, tyrosine, and tryptophan), or a protein, or any combination thereof. In some embodiments, the side group or the bulky moiety is covalently bound to a methylene group adjacent to the disulfide bond.

In some embodiments, the disulfide bond is located adjacent to the biological payload and/or to the protein carrier of the invention, wherein adjacent is as described herein. In some embodiments, the biological payload and/or to the protein carrier of the invention is bound to the linker via a disulfide bond. In some embodiments, the disulfide bond is proximal or adjacent to the protein carrier.

In some embodiments, the protein carrier of the invention (e.g., HSA) is bound to the linker via a disulfide bond. In some embodiments, the HSA comprises the amino acid sequence of DAHKSEVAHRFKDLGEENFKALVLIAFAQYLQQCPFEDHVKLVNEVTEFAKTCVA DESAENCDKSLHTLFGDKLCTVATLRETYGEMADCCAKQEPERNECFLQHKDDN PNLPRLVRPEVDVMCTAFHDNEETFLKKYLYEIARRHPYFYAPELLFFAKRYKAAF TECCQAADKAACLLPKLDELRDEGKASSAKQRLKCASLQKFGERAFKAWAVARL SQRFPKAEFAEVSKLVTDLTKVHTECCHGDLLECADDRADLAKYICENQDSISSKL KECCEKPLLEKSHCIAEVENDEMPADLPSLAADFVESKDVCKNYAEAKDVFLGMF LYEYARRHPDYSVVLLLRLAKTYETTLEKCCAAADPHECYAKVFDEFKPLVEEPQ NLIKQNCELFEQLGEYKFQNALLVRYTKKVPQVSTPTLVEVSRNLGKVGSKCCKH PEAKRMPCAEDYLSVVLNQLCVLHEKTPVSDRVTKCCTESLVNRRPCFSALEVDE TYVPKEFNAETFTFHADICTLSEKERQIKKQTALVELVKHKPKATKEQLKAVMDD FAAFVEKCCKADDKETCFAEEGKKLVAASQAALGL (SEQ ID NO: 1), or a fragment or homolog thereof. SEQ ID NO: 1 provides the sequence of HSA without a signal peptide.

In some embodiments, the HSA comprises a signal peptide. In some embodiments, the HSA is a fragment of HSA. In some embodiments, a fragment comprises at least 50, 60, 70, 80, 90, 95, 99 or 100% of HSA. Each possibility represents a separate embodiment of the invention. In some embodiments, the fragment comprises at least 50,100,150, 200, 250, 300, 350, 400, 450, 500, 550 or 600 amino acids from HSA. Each possibility represents a separate embodiment of the invention. In some embodiments, the amino acids are sequential amino acids. In some embodiments, the HSA is a homolog of HSA. In some embodiments, a homolog of HSA comprises an amino acid sequence with at least 70%, 75%, 80%, 85%, 90%, 95%, 97% or 99% homology to SEQ ID NO: 1. Each possibility represents a separate embodiment of the invention. In some embodiments, the HSA comprises an amino acid sequence with at least 70% homology to SEQ ID NO: 1. In some embodiments, the HSA consists of SEQ ID NO: 1 or a fragment or homolog thereof. In some embodiments, the HSA consists of an amino acid sequence with at least 70% homology to SEQ ID NO: 1. In some embodiments, the HSA consists of SEQ ID NO: 1. In some embodiments, the HSA comprises a free cysteine. In some embodiments, the free cysteine is cysteine C34. In some embodiments, a free cysteine is only a single free cysteine. In some embodiments, the linker of the invention is bound to C34 of HSA via a disulfide bond.

In some embodiments, fibrinogen is fibrinogen alpha chain (FGA) In some embodiments, the FGA comprises the amino acid sequence of ADSGEGDFLAEGGGVRGPRVVERHQSACKDSDWPFCSDEDWNYKCPSGCRMKG LIDEVNQDFTNRINKLKNSLFEYQKNNKDSHSLTTNIMEILRGDFSSANNRDNTYN RVSEDLRSRIEVLKRKVIEKVQHIQLLQKNVRAQLVDMKRLEVDIDIKIRSCRGSCS RALAREVDLKDYEDQQKQLEQVIAKDLLPSRDRQHLPLIKMKPVPDLVPGNFKSQ LQKVPPEWKALTDMPQMRMELERPGGNEITRGGSTSYGTGSETESPRNPSSAGSW NSGSSGPGSTGNRNPGSSGTGGTATWKPGSSGPGSTGSWNSGSSGTGSTGNQNPG SPRPGSTGTWNPGSSERGSAGHWTSESSVSGSTGQWHSESGSFRPDSPGSGNARPN NPDWGTFEEVSGNVSPGTRREYHTEKLVTSKGDKELRTGKEKVTSGSTTTTRRSC SKTVTKTVIGPDGHKEVTKEVVTSEDGSDCPEAMDLGTLSGIGTLDGFRHRHPDE AAFFDTASTGKTFPGFFSPMLGEFVSETESRGSESGIFTNTKESSSHHPGIAEFPSRG KSSSYSKQFTSSTSYNRGDSTFESKSYKMADEAGSEADHEGTHSTKRGHAKSRPV RDCDDVLQTHPSGTQSGIFNIKLPGSSKIFSVYCDQETSLGGWLLIQQRMDGSLNF NRTWQDYKRGFGSLNDEGEGEFWLGNDYLHLLTQRGSVLRVELEDWAGNEAYA EYHFRVGSEAEGYALQVSSYEGTAGDALIEGSVEEGAEYTSHNNMQFSTFDRDAD QWEENCAEVYGGGWWYNNCQAANLNGIYYPGGSYDPRNNSPYEIENGVVWVSF RGADYSLRAVRMKIRPLVTQ (SEQ ID NO: 2), or a fragment or homolog thereof. SEQ ID NO: 2 provides the sequence of fibrinogen without a signal peptide. In some embodiments, the fibrinogen comprises a signal peptide. In some embodiments, the signal peptide comprises or consists of MFSMRIVCLVLSVVGTAWT (SEQ ID NO: 3). In some embodiments, the FGA is a fragment of FGA. In some embodiments, a fragment comprises at least 50, 60, 70, 80, 90, 95, 99 or 100% of FGA. Each possibility represents a separate embodiment of the invention. In some embodiments, the fragment comprises at least 50, 100, 150, 200, 250, 300, 350, 400, 450, 500, 550, 600, 650, 700, 750, 800 or 850 amino acids from FGA. Each possibility represents a separate embodiment of the invention. In some embodiments, the amino acids are sequential amino acids. In some embodiments, the FGA is a homolog of FGA. In some embodiments, homology is sequence identity. In some embodiments, a homolog of FGA comprises an amino acid sequence with at least 70%, 75%, 80%, 85%, 90%, 95%, 97% or 99% homology to SEQ ID NO: 2. Each possibility represents a separate embodiment of the invention. In some embodiments, the FGA consists of SEQ ID NO: 2 or a fragment or homolog thereof. In some embodiments, the FGA consists of an amino acid sequence with at least 70% homology to SEQ ID NO: 2. In some embodiments, the FGA consists of SEQ ID NO: 2.

In some embodiments, fibrinogen is fibrinogen beta chain (FGB) In some embodiments, the FGB comprises the amino acid sequence of QGVNDNEEGFFSARGHRPLDKKREEAPSLRPAPPPISGGGYRARPAKAAATQKKV ERKAPDAGGCLHADPDLGVLCPTGCQLQEALLQQERPIRNSVDELNNNVEAVSQT SSSSFQYMYLLKDLWQKRQKQVKDNENVVNEYSSELEKHQLYIDETVNSNIPTNL RVLRSILENLRSKIQKLESDVSAQMEYCRTPCTVSCNIPVVSGKECEEIIRKGGETSE MYLIQPDSSVKPYRVYCDMNTENGGWTVIQNRQDGSVDFGRKWDPYKQGFGNV ATNTDGKNYCGLPGEYWLGNDKISQLTRMGPTELLIEMEDWKGDKVKAHYGGF TVQNEANKYQISVNKYRGTAGNALMDGASQLMGENRTMTIHNGMFFSTYDRDN DGWLTSDPRKQCSKEDGGGWWYNRCHAANPNGRYYWGGQYTWDMAKHGTDD GVVWMNWKGSWYSMRKMSMKIRPFFPQQ (SEQ ID NO: 19), or a fragment or homolog thereof. SEQ ID NO: 19 provides the sequence of fibrinogen without a signal peptide. In some embodiments, the fibrinogen comprises a signal peptide. In some embodiments, the signal peptide comprises or consists of MKRMVSWSFHKLKTMKHLLLLLLCVFLVKS (SEQ ID NO: 18). In some embodiments, the FGB is a fragment of FGB. In some embodiments, a fragment comprises at least 50, 60, 70, 80, 90, 95, 99 or 100% of FGB. Each possibility represents a separate embodiment of the invention. In some embodiments, the fragment comprises at least 50, 100, 150, 200, 250, 300, 350, 400, 450, or 480 amino acids from FGB. Each possibility represents a separate embodiment of the invention. In some embodiments, the amino acids are sequential amino acids. In some embodiments, the FGB is a homolog of FGB. In some embodiments, homology is sequence identity. In some embodiments, a homolog of FGB comprises an amino acid sequence with at least 70%, 75%, 80%, 85%, 90%, 95%, 97% or 99% homology to SEQ ID NO: 19. Each possibility represents a separate embodiment of the invention. In some embodiments, the FGB consists of SEQ ID NO: 19 or a fragment or homolog thereof. In some embodiments, the FGB consists of an amino acid sequence with at least 70% homology to SEQ ID NO: 19. In some embodiments, the FGB consists of SEQ ID NO: 19.

In some embodiments, fibrinogen is fibrinogen gamma chain (FGG) In some embodiments, the FGG comprises the amino acid sequence of YVATRDNCCILDERFGSYCPTTCGIADFLSTYQTKVDKDLQSLEDILHQVENKTSE VKQLIKAIQLTYNPDESSKPNMIDAATLKSRKMLEEIMKYEASILTHDSSIRYLQEI YNSNNQKIVNLKEKVAQLEAQCQEPCKDTVQIHDITGKDCQDIANKGAKQSGLYF IKPLKANQQFLVYCEIDGSGNGWTVFQKRLDGSVDFKKNWIQYKEGFGHLSPTGT TEFWLGNEKIHLISTQSAIPYALRVELEDWNGRTSTADYAMFKVGPEADKYRLTY AYFAGGDAGDAFDGFDFGDDPSDKFFTSHNGMQFSTWDNDNDKFEGNCAEQDG SGWWMNKCHAGHLNGVYYQGGTYSKASTPNGYDNGIIWATWKTRWYSMKKTT MKIIPFNRLTIGEGQQHHLGGAKQVRPEHPAETEYDSLYPEDDL (SEQ ID NO: 21), or a fragment or homolog thereof. SEQ ID NO: 21 provides the sequence of fibrinogen without a signal peptide. In some embodiments, the fibrinogen comprises a signal peptide.

In some embodiments, the signal peptide comprises or consists of MSWSLHPRNLILYFYALLFLSSTCVA (SEQ ID NO: 20). In some embodiments, the FGG is a fragment of FGG. In some embodiments, a fragment comprises at least 50, 60, 70, 80, 90, 95, 99 or 100% of FGG. Each possibility represents a separate embodiment of the invention. In some embodiments, the fragment comprises at least 50,100,150, 200, 250, 300, 350, 400, 450, or 480 amino acids from FGG. Each possibility represents a separate embodiment of the invention. In some embodiments, the amino acids are sequential amino acids. In some embodiments, the FGG is a homolog of FGG. In some embodiments, homology is sequence identity. In some embodiments, a homolog of FGG comprises an amino acid sequence with at least 70%, 75%, 80%, 85%, 90%, 95%, 97% or 99% homology to SEQ ID NO: 21. Each possibility represents a separate embodiment of the invention. In some embodiments, the FGG consists of SEQ ID NO: 21 or a fragment or homolog thereof. In some embodiments, the FGG consists of an amino acid sequence with at least 70% homology to SEQ ID NO: 21. In some embodiments, the FGG consists of SEQ ID NO: 21.

In some embodiments, fibrinogen is a mix of fibrinogen. In some embodiments, a mix is a mix of at least two of FGA, FGB and FGG. In some embodiments, a mix is a mix of all three of FGA, FGB and FGG. In some embodiments, the FGA, FGB and FGG are in a ratio such as is found in human blood. In some embodiments, blood is plasma. Fibrinogen from human plasma is commercially available such as from Sigma-Aldrich Cat. Number 341578. In some embodiments, the fibrinogen comprises a free cysteine. In some embodiments, the fibrinogen comprises a free lysine. Conjugation to fibrinogen can be performed as described herein or by any means known in the art. Conjugation can be random or site specific as described herein.

In some embodiments, the linker of the invention is or comprises a linear or a branched chain. In some embodiments, the linker of the invention is or comprises a backbone optionally comprising one or more said chain.

In some embodiments, the linker of the invention is a spacer (e.g., a natural and/or unnatural amino acid, alkyl, an amide bond, an ester bond, a thioester bond, a urea bond, including any derivative or a combination thereof). In some embodiments, the linker of the invention comprises a biocompatible polymer or a biocompatible moiety. In some embodiments, the biocompatible polymer is at least partially biodegradable. In some embodiments, the biocompatible polymer is or comprises a polyglycol ether, a polyester, a polyamide, a polyamino acid, a peptide and/or a derivative thereof or any combination thereof. In some embodiments, the polyglycol ether is or comprises polyethylene glycol (PEG). In some embodiments, the linker of the invention comprises PEG. In some embodiments, the linker of the invention comprises PEG characterized by Mn of between 100 and 5000 Da including any range between.

In some embodiments, the biocompatible moiety is or comprises an amide, an ester, a glycol, an amino acid, or any combination thereof.

In some embodiments, the polyamino acid or a derivative thereof comprises between 2 and 50 amino acids, between 4 and 50, between 5 and 50, between 5 and 50, between 4 and 20, between 4 and 30, between 4 and 40, between 5 and 20, between 5 and 30, between 5 and 40, between 6 and 50, between 6 and 30, between 6 and 40, between 6 and 20, between 8 and 50, between 8 and 30, between 8 and 20, between 8 and 40, including any range between.

The terms “peptide”, “polypeptide” and “protein” as used herein encompass native peptides, peptide derivatives such as beta peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

The term “derivative” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In addition, a peptide derivative can differ from the natural sequence of the peptide of the invention by chemical modifications including, but are not limited to, terminal-NH2 acylation, acetylation, or thioglycolic acid amidation, and by amidation of the terminal and/or side-chain carboxy group, e.g., with ammonia, methylamine, and the like. Peptides can be either linear, cyclic, or branched and the like, having any conformation, which can be achieved using methods known in the art.

In some embodiments, the linker of the invention further comprises a spacer (e.g., a natural and/or unnatural amino acid, alkyl, an amide bond, an ester bond, disulfide bond, a thioester bond, a urea bond, including any derivative or a combination thereof). In some embodiments, the linker of the invention further comprises a disulfide bond. In some embodiments, the linker of the invention comprises a click reaction product (e.g., a covalent linkage such as a cyclization reaction product, and/or a succinimide-thioether moiety formed via a click reaction).

Click reactions are well-known in the art and comprise inter alia Michael addition of maleimide and thiol (resulting in the formation of a succinimide-thioether); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction etc.

In some embodiments, the protein conjugate of the invention is represented by Formula 1:

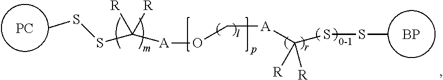

wherein PC represents the protein carrier (i.e., the masked protein carrier) of the invention; BP represents the biological payload of the invention; each r, and m independently represents an integer ranging from 0 to 10 including any range between; l represents an integer ranging from 1 to 10 including any range between; and p represents an integer ranging from 2 to 100 including any range between; each R independently represents the bulky moiety or H; each A independently represents one or more linkers, wherein each linker independently comprises any of: a heteroatom (e.g., O, N, NH, NR3, or S), a carbonyl derivative (e.g., —C(O)NH—, —C(O)O—, —C(O)—, —C(O)S—, —C(NR3)NR3-, —C(NR3)O—, —C(NR3)S—), a C1-C10 alkyl, a C1-C10 aminoalkyl, a C1-C10 alkoxy, a C1-C10 mercaptoalkyl, or a click reaction product including any combination thereof, or A is absent.

In some embodiments, the protein conjugate of the invention is represented by Formula 1:

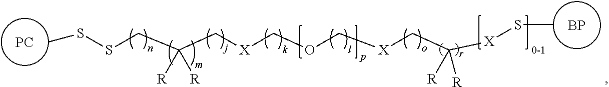

wherein PC represents the protein carrier (or charge masked moiety) of the invention; BP represents the biological payload of the invention; each j, k, r, o, n and m independently represents an integer ranging from 0 to 10 including any range between; 1 represents an integer ranging from 1 to 10 including any range between; and p represents an integer ranging from 2 to 100 including any range between; each R independently represents the bulky moiety or H; each X independently represents a heteroatom (e.g., O, N, NH, or S), a carbonyl derivative (e.g., —C(O)NH—, —C(O)O—, —C(O)—, —C(O)S—, —C(NH)NH—, —C(NH)O—, —C(NH)S—), a spacer (e.g., a C1-C10 alkyl, a C1-C10 aminoalkyl, a C1-C10 alkoxy, a C1-C10 mercaptoalkyl, or a click reaction product) or a combination thereof, or X is absent. In some embodiments, at least one R is methyl.

In some embodiments, the click reaction product comprises a moiety formed via a click reaction, wherein the click reaction is as described hereinabove. In some embodiments, the click reaction product comprises a product formed by any of: Michael addition of maleimide and thiol (resulting in the formation of a succinimide-thioether); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction, or any combination thereof.

In some embodiments, the protein conjugate of the invention is represented by Formula 1A:

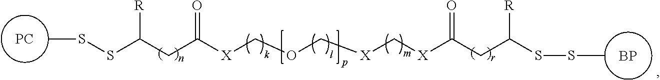

wherein R, n, k, 1, p, m, and r are as described herein, and wherein each X independently represents a heteroatom (e.g., O, N, NH, or S), a spacer (e.g., a C1-C10 alkyl, a C1-C10 aminoalkyl, a C1-C10 alkoxy, a C1-C10 mercaptoalkyl, or a click reaction product) or a combination thereof, or X is absent.

In some embodiments, the protein conjugate of the invention is represented by Formula A:

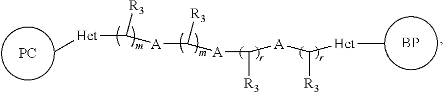

wherein PC represents the protein carrier of the invention (or charge masked moiety); BP represents the biological payload (i.e., the payload) of the invention; each r, and m independently represents an integer ranging from 0 to 10 including any range between; each R3 independently represents a substituent or H; Het represents a heteroatom, each independently selected from O, N, NH, and S; each A independently represent (i) a biocompatible moiety or a biocompatible polymer; and/or (ii) one or more linkers, wherein each linker independently comprises any of: a heteroatom (e.g., O, N, NH, or S), a carbonyl derivative (e.g., —C(O)NH—, —C(O)O—, —C(O)—, —C(O)S—, —C(NH)NH—, —C(NH)O—, —C(NH)S—), a C1-C10 alkyl, a C1-C10 aminoalkyl, a C1-C10 alkoxy, a C1-C10 mercaptoalkyl, or a click reaction product including any combination thereof, or A is absent. In some embodiments, at least one r or m is between 1 and 10, 1 and 3, 1 and 5, or 1, 2, 3, 4, 5, 6, or 10, including any range between.

In some embodiments, the protein conjugate of the invention is represented by Formula B:

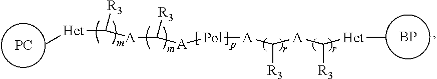

### or by Formula C:

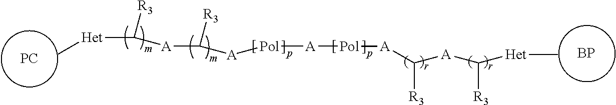

wherein PC represents the protein carrier of the invention; BP represents the biological payload of the invention; each r, and m independently represents an integer ranging from 0 to 10 including any range between; and p represents an integer ranging from 0 to 100 including any range between; Pol represent a biocompatible moiety or a biocompatible polymer; each R3 independently represents a substituent or H; Het represents a heteroatom, each independently selected from O, N, NH, and S; each A independently represents one or more linkers, wherein each linker independently comprises any of: a heteroatom (e.g., O, N, NH, or S), a carbonyl derivative (e.g., —C(O)NH—, —C(O)O—, —C(O)—, —C(O)S—, —C(NH)NH—, —C(NH)O—, —C(NH)S—), a C1-C10 alkyl, a C1-C10 aminoalkyl, a C1-C10 alkoxy, a C1-C10 mercaptoalkyl, or a click reaction product including any combination thereof, or A is absent. In some embodiments, the protein conjugate of the invention is represented by any one of Formulae A-C wherein the linker has a length of at least 5, at least 10, at least 15, at least 20, at least 30, at least 5, at least 100, at least 300, at least 500 between 5 and 500, between 5 and 100, between 10 and 100, between 5 and 50, between 50 and 100, between, 100 and 500 atomic bonds, including any range between. In some embodiments, at least one p is between 1 and 100, 1 and 20, 10 and 100, 2 and 20, 3 and 20, 3 and 15, 10 and 20, 20 and 50, 50 and 100, or 1, 2, 3, 4, 5, 6, 10, 11, 12, 15, or 20 including any range between.

In some embodiments, the protein conjugate of the invention is represented by Formula 1:

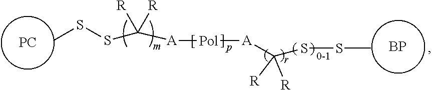

wherein PC, BP and Pol are as described herein; each r, and m independently represents an integer ranging from 0 to 10 including any range between; 1 represents an integer ranging from 1 to 10 including any range between; and p represents an integer ranging from 2 to 100 including any range between; each R independently represents the bulky moiety or H; each A independently represents one or more linkers, wherein each linker independently comprises any of: a heteroatom (e.g., O, N, NR₃, or S), a carbonyl derivative (e.g., —C(O)NH—, —C(O)O—, —C(O)—, —C(O)S—, —C(NR₃)NR₃-, —C(NR₃)O—, —C(NR₃)S—), a C1-C10 alkyl, a C1-C10 aminoalkyl, a C1-C10 alkoxy, a C1-C10 mercaptoalkyl, or a click reaction product including any combination thereof, or A is absent.

In some embodiments, the protein conjugate of the invention is represented by Formula:

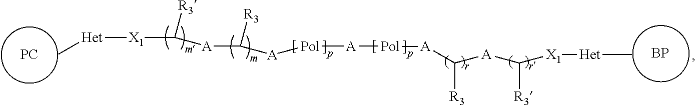

wherein PC, BP, Het, A, Pol, and p are as described herein; each r, r′, m and m′ independently represents an integer ranging from 0 to 10 including any range between; each R3 and R3′ independently represents one or more bulky moiety, one or more substituents, or H; and X1 represents a heteroatom (e.g., O, N, NR₃, or S), a carbonyl derivative (e.g., —C(O)NH—, —C(O)O—, —C(O)—, —C(O)S—, —C(NR₃)NR₃-, —C(NR₃)O—, —C(NR₃)S—), or a click reaction product including any combination thereof, and if Het is S, then at least one X1 is S. In some embodiments, if Het and X1 are both S, then at least one R₃ is one or more bulky moiety; and at least one of m‘ and r’ is not 0.

In some embodiments, the protein conjugate of the invention is represented by Formula:

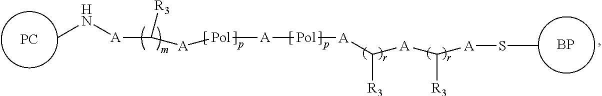

wherein PC, BP, Het, A, Pol, R₃, r, m, and p are as described herein, as allowed by valency. In some embodiments, if Het and X1 are both S, then at least one R₃ is one or more bulky moiety; and at least one of m‘ and r’ is not 0.

In some embodiments, the protein conjugate of the invention is represented by Formula:

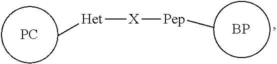

wherein Het comprises S or NH, wherein X represents a carbonyl derivative, a click reaction product, or is a bond; and wherein Pep represents a peptide. In some embodiments, the peptide is bound to the C-terminus of BP. In some embodiments, Het is S, and the peptide is bound to PC via cysteine (e.g., a C-terminal cysteine).

In some embodiments, the protein conjugate of the invention is represented by any one of Formulae:

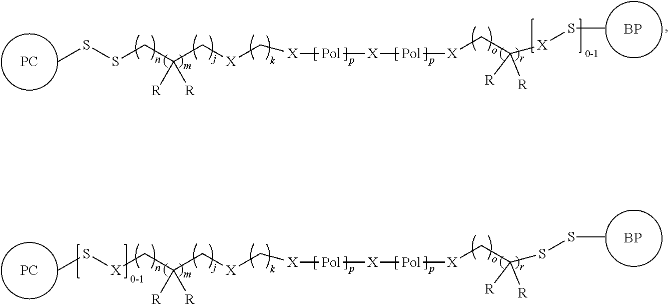

wherein PC, BP, and Pol, are as described herein; each j, k, r, o, n and m independently represents an integer ranging from 0 to 10 including any range between; 1 represents an integer ranging from 1 to 10 including any range between; and p represents an integer ranging from 2 to 100 including any range between; each R independently represents the bulky moiety or H; each X independently represents a heteroatom (e.g., O, N, NH, or S), a carbonyl derivative (e.g., —C(O)NH—, —C(O)O—, —C(O)—, —C(O)S—, —C(NH)NH—, —C(NH)O—, —C(NH)S—), a spacer (e.g., a C1-C10 alkyl, a C1-C10 aminoalkyl, a C1-C10 alkoxy, a C1-C10 mercaptoalkyl, or a click reaction product) or a combination thereof, or X is absent. In some embodiments, at least one R is methyl. In some embodiments, Pol represents a peptide, an amino acid or a dehydrated derivative thereof, PEG, or —CH₂—CH₂—O—. In some embodiments, a dehydrated derivative of the amino acid encompasses:

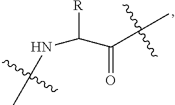

wherein the wavy bonds represent an attachment point to the linker or to the subsequent monomer, and wherein R presents an amino acid side chain (optionally wherein R and NH are interconnected so as to form a ring resulting in a deprotonation of NH, such as in proline).

In some embodiments, the click reaction product comprises a moiety formed via a click reaction, wherein the click reaction is as described hereinabove. In some embodiments, the click reaction product comprises a product formed by any of: Michael addition of maleimide and thiol (resulting in the formation of a succinimide-thioether); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction, or any combination thereof. In some embodiments, the click reaction product is succinimide-thioether.

In some embodiments, the conjugate of the invention is represented by any of the above-described Formulae, wherein X or X1 are click reaction product, optionally wherein the click reaction product is succinimide-thioether.

In some embodiments, the conjugate of the invention is represented by Formula 2 below:

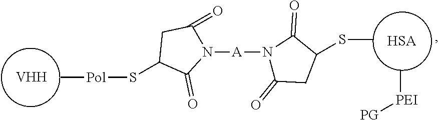

wherein Pol represent a biocompatible moiety or a biocompatible polymer (e.g. a peptide comprising a C-terminal cysteine); and A represents a spacer, or any of: a heteroatom (e.g., O, N, NH, or S), a carbonyl derivative (e.g., —C(O)NH—, —C(O)O—, —C(O)—, —C(O)S—, —C(NH)NH—, —C(NH)O—, —C(NH)S—), a C1-C10 alkyl, a C1-C10 aminoalkyl, a C1-C10 alkoxy, a C1-C10 mercaptoalkyl, PEG, alkyl-PEG, alkyl-PEG-alkyl, alkylamide-PEG-alkylamide, or a click reaction product including any combination thereof, or A is absent; and wherein alkyl is a C1-C10 alkyl optionally comprising one or more of: (i) one or more heteroatoms, (ii) one or more carbonyl derivatives, (iii) one or more disulfide bonds, (iv) one or more click reaction product.

In some embodiments, the protein conjugate of the invention is represented by Formula 1A:

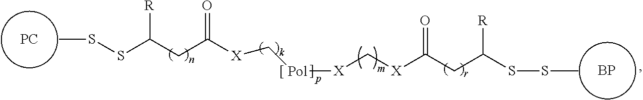

wherein R, n, k, l, p, m, Pol, and r are as described herein, and wherein each X independently represents a heteroatom (e.g., O, N, NH, or S), a spacer (e.g., a C1-C10 alkyl, a C1-C10 aminoalkyl, a C1-C10 alkoxy, a C1-C10 mercaptoalkyl, or a click reaction product) or a combination thereof, or X is absent. In some embodiments, Pol represents an amino acid or a dehydrated derivative thereof, or —CH₂—CH₂—O—.

In some embodiments, the linker of the invention is bound to the HSA via a disulfide bond. In some embodiments, the linker of the invention is covalently bound to an amino group, or to a thiol group of the biological payload of the invention. In some embodiments, each HSA is bound to a single biological payload. In some embodiments, each HSA is bound to a plurality of biological payloads.

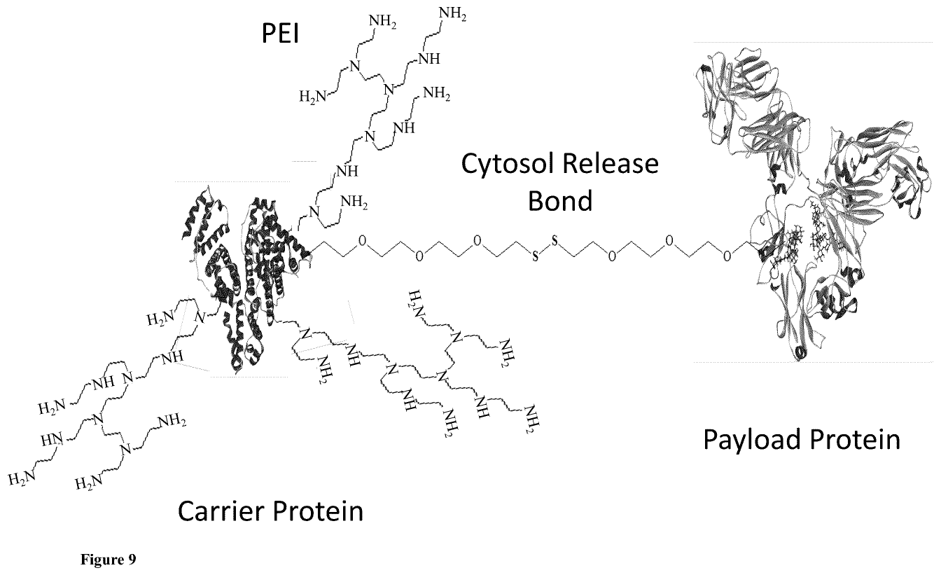

In some embodiments, the linker of the invention is bound to the HSA via a disulfide bond. In some embodiments, the linker of the invention is covalently bound to an amino group, or to a thiol group of the biological payload of the invention. In some embodiments, each HSA is bound to a single biological payload. In some embodiments, each HSA is bound to a plurality of biological payloads. Exemplary protein conjugates of the invention are represented by FIG. 9, and in the Examples section.

In some embodiments, the protein conjugate of the invention is substantially stable in a biological fluid for at least 2 h, at least 10 h, at least 24 h, at least 48 h, at least 72 h, including any range between.

In some embodiments, at least 25%, at least 50%, at least 75%, at least 90% of the protein conjugate of the invention is substantially stable, including any range between.

As used herein, the term “stable” refers to the ability of the protein conjugate or linker of the invention to maintain: (i) its chemical integrity (e.g., substantially devoid of cleavage and or deprotection), and (ii) its initial concentration and/or biological activity within a tissue and/or a biological fluid of a subject.

In some embodiments, the protein conjugate of the invention and/or the protein carrier of the invention is characterized by an increased stability, compared to a control (e.g., an analogous protein conjugate or protein carrier devoid of protected amines). In some embodiments, the protein conjugate of the invention and/or the protein carrier of the invention is characterized by an increased stability within a biological fluid and/or within a tissue (e.g., a healthy tissue having a pH of above 7), compared to a control; wherein increased is by at least 10%, at least 50%, at least 100%, at least 500%, at least 1000%, at least 10.000%, or more, compared to the control.

In some embodiments, the protein conjugate of the invention and/or the protein carrier of the invention is characterized by an increased accumulation within a target tissue having a pH value of less than 7, less than 6.8, less than 6.5; wherein increased is by at least 10%, at least 50%, at least 100%, at least 500%, at least 1000%, at least 10.000%, or more, compared to a control (e.g., an analogous protein conjugate or protein carrier devoid of the protecting group).

In some embodiments, the target tissue comprises a cancer tissue, an inflamed tissue, or both. In some embodiments, the target tissue comprises cancer. In some embodiments, the target tissue comprises inflammation. In some embodiments, the target tissue is a cancer. In some embodiments, the cancer is a solid cancer. In some embodiments, the target tissue is inflamed tissue.

### Targeting Moiety

In some embodiments, the protein conjugate further comprises a targeting moiety. As used herein, the term “targeting moiety” refers to any molecule that is able to specifically bind to a target protein. In some embodiments, the targeting moiety binds to a protein expressed on the surface of a target cell. In some embodiments, the protein is a surface protein. In some embodiments, the protein is a receptor. In some embodiments, the protein is a cancer specific antigen. In some embodiments, the protein is a surface marker for the target cell. In some embodiments, the target cell is a target cell type. In some embodiments, the cell type is a disease cell type. In some embodiments, binding is specifically binding. In some embodiments, specific binding to a target comprises not substantially binding to another target. In some embodiments, substantially is significantly. In some embodiments, none substantially binding is at most 0.001, 0.005, 0.01, 0.05, 0.1, 0.5, 1, 2, 3, 5, 7, or 10% binding to another target protein. Each possibility represents a separate embodiment of the invention.

The term “moiety”, as used herein, relates to a part of a molecule that may include either whole functional groups or parts of functional groups as substructures. The term “moiety” further means part of a molecule that exhibits a particular set of chemical and/or pharmacologic characteristics which are similar to the corresponding molecule. In this case the characteristic is binding to a target protein.

In some embodiments, the targeting moiety is an antigen binding molecule. In some embodiments, the antigen binding molecule is an antigen binding molecule that binds a surface target. In some embodiments, a target is a protein. In some embodiments, the targeting moiety is selected from a single chain antibody, a single domain antibody, a variable heavy homodimer (VHH), a nanobody, an immunoglobulin novel antigen receptor (IgNAR), a designed ankyrin repeat protein (DARPin) and an antibody mimetic protein. In some embodiments, the targeting moiety is a VHH.

In some embodiments, the targeting moiety modulates the target protein. In some embodiments, modulating comprises activating the target protein. In some embodiments, the modulating comprises inhibiting the target protein. In some embodiments, the targeting moiety is an agonist of the target protein. In some embodiments, the targeting moiety is an antagonist of the target protein.

In some embodiments, the targeting moiety is conjugated to the protein carrier. In some embodiments, conjugated is conjugated by a linker. In some embodiments, the targeting moiety is conjugated to the biological payload. In some embodiments, the linker is a branched linker that conjugates the targeting moiety, the biological payload and the protein carrier. In some embodiments, the targeting moiety and biological payload are comprised in a single polypeptide. In some embodiments, a single polypeptide is a single chain. In some embodiments, the targeting moiety and the biological payload are separated by a linker. In some embodiments, the targeting moiety is N-terminal to the biological payload. In some embodiments, the targeting moiety is C-terminal to the biological payload. In some embodiments, the targeting moiety is at the N-terminus of the polypeptide. In some embodiments, the targeting moiety is at the C-terminus of the polypeptide. In some embodiments, targeting moiety is separated from the C-terminus by a C-terminal cysteine residue. In some embodiments, targeting moiety is separated from the C-terminus by a C-terminal linker.

### Kit

In another aspect, there is provided a kit comprising the protein carrier covalently bound to a first moiety and the biological payload, wherein the first moiety is characterized by a reactivity to the biological payload, and wherein the protein carrier comprises the charge masked moiety of the invention. In some embodiments, the biological payload is covalently bound to a second moiety, wherein the first moiety and the second moiety have a reactivity to each other (e.g., via a click reaction). In some embodiments, the kit further comprises the PG.

In some embodiments, the protein carrier covalently bound to a first moiety is represented by Formula 2:

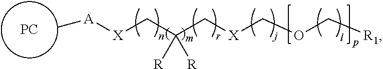

### or by Formula 2A:

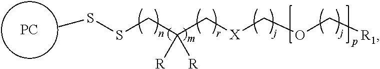

wherein R, n, j, l, p, m, and r are as described herein, wherein A is or comprises a heteroatom selected from O, NR, and S, and wherein R1 represents the first moiety.

In some embodiments, the biological payload covalently bound to a second moiety is represented by Formula 3:

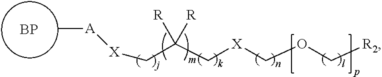

### or by Formula 4:

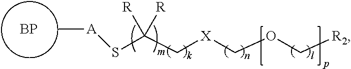

wherein R, X, j, k, l, n, m, and p are as described hereinabove, wherein A is or comprises a heteroatom selected from O, NR, and S, and wherein R2 represents the second moiety.

In some embodiments, the first moiety or the second moiety is or comprises 1,3-nitrone, azide, a diene, tetrazine, an active ester (e.g., thio-ester, a pentofluorophenyl ester, a N-hydroxysuccinimide ester), an acyl halide, a chloroformate, an anhydride, an aldehyde, an epoxide, an isocyanate, an isothiocyanate, a maleimide, a carbonate, a sulfonyl chloride, iodoacetamide, an acyl azide, an imidoester, a vinyl sulfone, ortho-pyridyl-disulfide, or any combination thereof.

In some embodiments, the first moiety or the second moiety is or comprises a nucleophilic group (e.g., an amine, a thiol, a phosphine, a hydroxyl), a dienophile, an alkene, and an alkyne (e.g., acetylene, dibenzyl cyclooctyne, etc.), or any combination thereof.

In some embodiments, the kit of the invention comprises the biological payload covalently bound to a linker comprising a functional group having reactivity to the HSA (e.g., to a cysteine or to a lysine thereof); and HSA. In some embodiments, the kit of the invention comprises the HSA covalently bound to a linker comprising a functional group having reactivity to the biological payload (e.g., to a cysteine or to a lysine thereof); and the biological payload. In some embodiments, the functional group is or comprises any of iodoacetamide, an active ester, ortho-pyridyldisulfide, a maleimide, or a combination thereof.

In some embodiments, the conjugate is a blood-stable conjugate. In some embodiments, the conjugate is a cell-penetrating conjugate. In some embodiments, the conjugate is a masked conjugate. In some embodiments, the conjugate is a conjugate that can be masked. In some embodiments, the conjugate is a cell membrane crossing conjugate. In some embodiments, the conjugate is able to enter cells. In some embodiments, the conjugate is capable of endosome escape. In some embodiments, the conjugate is capable of intracellular delivery of a payload. In some embodiments, intracellular delivery is cytoplasmic delivery. In some embodiments, intracellular delivery comprises dissociation of the carrier from the payload. In some embodiments, the conjugate is configured to dissociate in the cytoplasm. In some embodiments, the dissociation is dissociation of the carrier from the payload. In some embodiments, the conjugate is for use in modulating an intracellular target. In some embodiments, the conjugate is for use in effecting an intracellular target. In some embodiments, the conjugate is for use in interacting with an intracellular target.

### Method

In another aspect, there is provided a method of producing a charge masked protein conjugate, the method comprising: providing a biological agent that binds an intracellular target; providing a protein carrier covalently bound to a cell penetrating moiety, the cell penetrating moiety comprises a plurality of amine groups; providing the biological payload and the protein carrier under conditions sufficient for covalently binding said biological payload to the protein carrier via a linker to produce a protein conjugate; providing the protein carrier under conditions sufficient for protecting at least a portion of the amine groups by a protecting group capable of undergoing cleavage (deprotection) at a pH value of less than 7, to obtain the charge masked protein conjugate comprising protected amine groups.

In some embodiments, the method further comprises determining stability of the linker in a biological fluid and in cytoplasmic conditions; and selecting a charge masked protein conjugate comprising a linker that is stable in the biological fluid and unstable in the cytoplasmic conditions; thereby producing the charge masked protein conjugate capable of binding an intracellular target. In some embodiments, the step of protecting at least a portion of the amine groups is performed (i) prior to performing the step of production of the protein conjugate; or (ii) subsequent to the step of production of the protein conjugate. In some embodiments, providing the protein carrier under conditions sufficient for protecting occurs before the binding the biological payload to the protein carrier. In some embodiments, providing the protein carrier under conditions sufficient for protecting occurs after the binding the biological payload to the protein carrier. In some embodiments, the providing is providing the protein carrier unlinked. In some embodiments, the providing is providing the protein conjugate. It will be understood by a skilled artisan that when the protein conjugate is protected basic residues on the payload and linker will also be protected and thus the full conjugate is protected. In some embodiments, the method is for producing the charge masked protein conjugate of the invention. In some embodiments, the terms “charge masked protein conjugate” and “protein conjugate” are used herein interchangeably. In some embodiments, the selecting is selecting a charge masked protein conjugate that is more stable in the biological fluid that in the cytoplasmic conditions. In some embodiments, more stable is at least 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 250, 300, 350, 400, 450, or 500% more stable. Each possibility represents a separate embodiment of the invention.

In another aspect, there is provided a method of producing a charge masked protein conjugate, the method comprising: providing a biological agent that binds an intracellular target; providing a protein carrier covalently bound to a cell penetrating moiety, the cell penetrating moiety comprises a plurality of amine groups, and subsequently providing the protein conjugate under conditions sufficient for protecting at least a portion of the amine groups by a protecting group capable of undergoing cleavage (deprotection) at a pH value of less than 7, to obtain a masked protein carrier comprising protected amine groups; providing the biological payload and the masked protein carrier under conditions sufficient for covalently binding said biological payload to the protein carrier via a linker.

In another aspect, there is provided a method of producing a charge masked protein conjugate, the method comprising: providing a biological agent that binds an intracellular target; providing a masked protein carrier covalently bound to a cell penetrating moiety, wherein the cell penetrating moiety comprises a plurality of protected amine groups; providing the biological payload and the protein carrier under conditions sufficient for covalently binding said biological payload to the protein carrier via a linker comprising at least one bio-cleavable bond to produce the protein conjugate.

In another aspect, there is provided a method of producing a charge masked protein, the method comprising: providing a biological agent that binds an intracellular target, binding the biological agent to a cell penetrating moiety, and providing the biological agent bound to a cell penetrating moiety under conditions sufficient for protecting at least a portion of the cell penetrating moiety. In some embodiments, the cell penetrating moiety comprises a plurality of amine groups. In some embodiments, at least a portion of the amines groups are protected. In some embodiments, the protecting group is capable of undergoing cleavage (deprotection at a pH value of less than 7.

In another aspect there is provided a method of producing a charge masked protein, the method comprising: providing a biological agent that binds an intracellular target, and binding the biological agent to a cell penetrating moiety, wherein the cell penetrating moiety comprises a plurality of protected amine groups.

In some embodiments, the charge masked protein conjugate is capable of binding to an intracellular target. In some embodiments, the charge masked protein conjugate is able to enter a cytoplasm of a cell. In some embodiments, the charge masked protein conjugate is capable of intracellular delivery of a biological agent. In some embodiments, the charge masked protein conjugate enables intracellular delivery of a biological agent. In some embodiments, the charge masked protein conjugate is capable of modulating an intracellular target. In some embodiments, the charge masked protein conjugate is configured to modulate an intracellular target. In some embodiments, the biological agent is devoid of a disulfide bond. In some embodiments, the biological agent is devoid of a disulfide bond that is required for the structure of the biological agent. In some embodiments, the biological agent is devoid of a disulfide bond that is required for the function of the biological agent. In some embodiments, the biological agent is devoid of a disulfide bond that is required for the binding of the biological agent. In some embodiments, the biological agent is devoid of a disulfide bond that when cleaved diminishes binding. In some embodiments, binding is binding to the intracellular target.

In some embodiments, the charge masked protein conjugate is a therapeutic agent. In some embodiments, the therapeutic agent is a biological therapeutic agent. In some embodiments, the therapeutic agent is a biologic. In some embodiments, the therapeutic agent is an agent against the intracellular target. In some embodiments, the therapeutic agent targets the intracellular target. In some embodiments, the therapeutic agent modulates the intracellular target.

In some embodiments, there is a method of synthesizing the charge masked protein conjugate of the invention. In some embodiments, the method comprises providing the kit of the invention and reacting the first moiety and the second moiety (e.g., under suitable conditions, optionally comprising a metal-based catalyst and/or a UV-, thermal-irradiation).

In some embodiments, the method of synthesizing the charge masked protein conjugate of the invention comprises: (i) providing the protein carrier covalently bound to the first moiety, and the biological payload covalently bound to the second moiety, wherein the first moiety and the second moiety have a reactivity to each other (e.g. via a click reaction, via thiol-maleimide linkage formation, via amine to active ester coupling, via S—S bond formation such as by reacting SPDP or a nitro-SPDP with a thiol); and (ii) providing the protein carrier and the biological payload under conditions suitable for a reaction between the first moiety and the second moiety, thereby synthesizing the protein conjugate; and (iii) subsequently reacting the protein conjugate with a PG precursor under conditions suitable for protecting at least a portion of the amines with the PG, thereby obtaining the charge masked protein conjugate of the invention.

In another aspect, there is provided a method of producing a charge masked protein conjugate, the method comprising: (i) providing a masked protein carrier covalently bound to the first moiety, and the biological payload covalently bound to the second moiety, and (ii) providing the masked protein carrier and the biological payload under conditions suitable for a reaction, thereby synthesizing the protein conjugate; wherein the masked protein carrier is synthesized by reacting the protein conjugate with a PG precursor under conditions suitable for protecting at least a portion of the amines with the PG.

In some embodiments, the conditions suitable for protecting comprise reaction conditions suitable for reacting an amine with the PG precursor, thereby obtaining a protected amine. In some embodiments, the conditions suitable for protecting comprise a neutral or basic pH; a temperature of at least −10° C., at least 0° C., at least 10° C., at least 20° C., at least 50° C., including any range between; and 90 and at least 10, at least 20, at least 50, at least 100 molar equivalents, at least 300 molar equivalents, at least 500 molar equivalents, of the PG precursor relative to the protein conjugate, including any range between.

In some embodiments, the steps (i) to (iii) of the method are performed in a solution (e.g., comprising an organic solvent, an aqueous solvent or a combination thereof).

In some embodiments, the first moiety and the second moiety are as described herein. In some embodiments, reaction comprises click reaction.

In some embodiments, the method comprises: (i) providing the protein carrier covalently bound to a linker comprising a functional group having reactivity to the biological payload (e.g., to a cysteine or to a lysine thereof) and the biological payload; (ii) reacting the functional group with the biological payload, thereby synthesizing the protein conjugate; and (iii) reacting the protein conjugate with the PG precursor, to obtain the charge masked protein conjugate.

In some embodiments, the method comprises: (i) providing the biological payload covalently bound to a linker comprising a functional group having reactivity to the protein carrier (e.g., to a cysteine or to a lysine thereof) and the protein carrier; (ii) reacting the functional group with the protein carrier, thereby synthesizing the protein conjugate; and (iii) protecting the amines of the cell penetrating moiety with the PG under conditions described herein, to obtain the charge masked protein conjugate of the invention.

In some embodiments, the method comprises testing cell penetrance of the masked conjugate. In some embodiments, the method comprises testing function of the biological payload upon delivery to a target cell. In some embodiments, the method comprises testing biodistribution of the masked conjugate. In some embodiments, the method comprises testing in vivo function of the biological payload in target cells. In some embodiments, the method comprises determining stability of the linker in a biological fluid and in cytoplasmic conditions. In some embodiments, the method comprises selecting a charge masked protein conjugate comprising a linker that is stable in the biological fluid and unstable in the cytoplasmic conditions. In some embodiments, the method comprises determining stability of the protected amine groups at biological pH and acidic pH. In some embodiments, biological pH is neutral pH. In some embodiments, biological pH is neutral or basic pH. In some embodiments, biological pH is a pH of about 7.4. In some embodiments, acidic pH is a pH of about 6.8. In some embodiments, acidic pH is a pH below 7. In some embodiments, acidic pH is a pH at or below 6.8 pH. In some embodiments, the method comprises selecting a charge masked protein conjugate comprising protected amine groups that are stable at biological pH and unstable at acidic pH. In some embodiments, the method comprises selecting a protecting group that is cleaved (deprotected) at a pH below 7. In some embodiments, the method comprises selecting a biological agent that binds to an intracellular target. In some embodiments, selecting comprises determining or measuring that the biological agent binds an intracellular target. Methods of performing such testing are provided hereinbelow and any such testing may be performed.

In some embodiments, the method further comprises selecting a targeting moiety. In some embodiments, the method further comprises selecting a moiety that binds to a protein of interest on the surface of a target cell. In some embodiments, a target cell is a cell of interest. In some embodiments, a target cell is a disease cell. In some embodiments, the method comprises conjugating the selected targeting moiety to the biological payload. In some embodiments, the method comprises conjugating the selected targeting moiety to the protein carrier. In some embodiments, the method comprises conjugating the selected targeting moiety to the masked protein carrier. In some embodiments, the method comprises conjugating the selected targeting moiety to the protein conjugate. In some embodiments, the method comprises conjugating the selected targeting moiety to the masked protein conjugate. In some embodiments, the conjugating is via a linker. In some embodiments, the conjugating is constructing a single polypeptide comprises the targeting moiety and the biological payload. In some embodiments, constructing comprises inserting the targeting moiety into the biological payload.

In some embodiments, the method comprises testing the binding of the targeting moiety to the target protein. In some embodiments, the method comprises testing the binding of the masked protein conjugate to the target protein. In some embodiments, to the target protein is to a cell expressing the target protein on its surface. In some embodiments, testing binding is testing specific binding. In some embodiments, testing comprises testing a lack of binding to a cell that does not comprise the target protein on its surface. Methods of performing such testing are provided hereinbelow and any such testing may be performed.

In another aspect, there is provided a protein conjugate produced by a method of the invention.

In another aspect, there is provided a protein produced by a method of the invention.

### Compositions

In another aspect, there is provided a pharmaceutical composition comprising a protein conjugate of the invention.

In another aspect, there is provided a pharmaceutical composition comprising a protein of the invention.

In some embodiments, the pharmaceutical composition comprises a pharmaceutically acceptable carrier, excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, glycols, such as propylene glycol, polyols such as glycerin, sorbitol, mannitol and polyethylene glycol; esters such as ethyl oleate and ethyl laurate, pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, stearic acid, magnesium stearate, calcium sulfate, polyols, pyrogen-free water, isotonic saline, phosphate buffer solutions, as well as other non-toxic pharmaceutically compatible substances used in other pharmaceutical formulations. Wetting agents and lubricants such as sodium lauryl sulfate, as well excipients, stabilizers, antioxidants, and preservatives may also be present. Any non-toxic, inert, and effective carrier may be used to formulate the compositions contemplated herein.

The carrier may comprise, in total, from about 0.1% to about 99.99999% by weight of the pharmaceutical compositions presented herein.

In some embodiments, the pharmaceutical composition comprises a therapeutically effective amount of the protein conjugate of the invention. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the pharmaceutical composition is formulated for systemic administration. In some embodiments, the pharmaceutical composition is formulated for local administration. In some embodiments, the pharmaceutical composition is formulated for intravenous administration. In some embodiments, the pharmaceutical composition is formulated for administration to a subject.

In some embodiments, the pharmaceutical composition is a slow-release compositions. In some embodiments, the linker is devoid of a bio cleavable bond and the composition is a slow-release composition. In some embodiments, slow release comprises payload delivery at least 1, 2, 3, 4, 5, 6, 7, 8, 9, 10, 11, 12, 13 or 14 days after administration. Each possibility represents a separate embodiment of the invention. In some embodiments, slow release comprises payload delivery at least 1 day after administration. In some embodiments, slow release comprises payload delivery at least 3 days after administration. In some embodiments, slow release comprises payload delivery at least 5 days after administration.

In some embodiments, the compounds of the present invention can exist in free form for treatment, or as a pharmaceutically acceptable salt.

As used herein, the term “pharmaceutically acceptable salt” refers to any non-toxic salt of a compound of the present invention that, upon administration to a subject, e.g., a human, is capable of providing, either directly or indirectly, a compound of this invention or an inhibitorily active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

Additional examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C1-4alkyl)₄ salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

In some embodiments, the term “one or more” refers to any numerical value selected form of 1, 2, 3, 4, 5, or 6. In some embodiments, the heteroatom comprises any of N, O, NH, or S.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute.

Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a hydrogen by 18F, or the replacement of a carbon by a 13C- or 14C-enriched carbon are within the scope of this invention. Such compounds are useful, for example, as imaging probes.

### Method of Use

In another aspect, there is provided a method of binding an intracellular target, the method comprising contacting a cell expressing the intracellular target with a protein conjugate of the invention, protein of the invention or a pharmaceutical composition of the invention, thereby binding the intracellular target.

In some embodiments, the method is a method of modulating the intracellular target. In some embodiments, the biological payload binds the intracellular target. In some embodiments, the biological payload modulates the intracellular target. In some embodiments, modifying is agonizing. In some embodiments, the biological payload is an agonist of the intracellular target. In some embodiments, modifying is antagonizing. In some embodiments, the biological payload is an antagonist. Molecules that modulate, (i.e., antagonize or agonize) are well known in the art and any such molecule may be employed. In some embodiments, the biological target is specific to the intracellular target.

In some embodiments, the method is a method of detecting the intracellular target. In some embodiments, the protein conjugate comprises a detectable tag. In some embodiments, the tag is a detectable moiety. In some embodiments, the method further comprises detecting the protein conjugate. In some embodiments, the method further comprises detecting the detectable tag. In some embodiments, the detectable tag is a fluorescent tag. Detectable tags and moieties are well known in the art and include, for non-limiting example, a fluorophore (e.g., GFP, RFP, YFP, luciferase and the like), a radioactive tag, and a colored tag). Any such known tag may be employed.

In some embodiments, the cell is within a subject. In some embodiments, the subject is a mammal. In some embodiments, the subject is a human. In some embodiments, the subject suffers from a disease or condition. In some embodiments, the disease or condition is treatable by contacting the intracellular target. In some embodiments, the disease or condition is treatable by modulating the intracellular target. In some embodiments, the disease or condition is treatable by agonizing the intracellular target. In some embodiments, the disease or condition is treatable by antagonizing the intracellular target. In some embodiments, the subject is in need of modulating the intracellular target. In some embodiments, the subject is in need of treatment. In some embodiments, the subject is a subject in need thereof.

In some embodiments, the method comprises administering to the subject a protein conjugate of the invention. In some embodiments, the method comprises administering to the subject a pharmaceutical composition of the invention.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, intratumoral or intraperitoneal.

The dosage administered will be dependent upon the age, health, and weight of the recipient, kind of concurrent treatment, if any, frequency of treatment, and the nature of the effect desired.

In some embodiments, the disease or condition is cancer. In some embodiments, the disease or condition is inflammation. In some embodiments, the disease or condition is ischemia. In some embodiments, the intracellular target is an oncogene and the biological payload is an antagonist. In some embodiments, the intracellular target is a tumor suppressor and the biological payload is an agonist.

In some embodiments, the contacting is not in the presence of an agent designed to induce penetration of the protein conjugate into a cell. In some embodiments, the agent designed to induce penetration is an agent other than the carrier protein. In some embodiments, another method of inducing cell penetration other the method of the invention is not employed. In some embodiments, the contacting persists is at least 1, 2, 3, 4, 5, 6, 7, 8, 9, 10, 11, 12, 13 or 14 days after the administration. Each possibility represents a separate embodiment of the invention. In some embodiments, the contacting persists at least 1 day after administration. In some embodiments, the contacting persists at least 3 days after administration. In some embodiments, the contacting persists at least 5 days after administration. In some embodiments, persistent contacting after administration is slow release of the payload/therapeutic.

In some embodiments, cells of the disease or condition express the target protein. In some embodiments, a protein conjugate comprising a targeting moiety is used to treat a disease or condition characterized by the expression of the target protein on cells of the disease or condition. In some embodiments, the target protein is a marker of the disease or condition. In some embodiments, the disease is cancer and the target protein is a cancer specific antigen. Cancer specific antigens and antigen binding molecules that bind them are well known in the art and any such molecule can be used in the method of the invention. In some embodiments, the cancer specific antigen is prostate specific membrane antigen (PSMA).

As used herein, the term “alkyl” describes an aliphatic hydrocarbon including straight chain and branched chain groups and usually comprising between 1 and 30, or between 1 and 10 carbon atoms. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein, or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N) NR″— linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C3-8 heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, Cl-6-haloalkyl, Cl-6-hydroxyalkyl, Cl-6-aminoalkyl, Cl-6-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, 0-C1-6-alkyl, C1-6-alkyl, hydroxy-C1-6-alkyl and amino-Cl-6-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

In some embodiments, the term “substituted” encompasses one or more substituents covalently bound to the functional group and/or to the molecule. In some embodiments, the substituent comprises one or more substituents, each independently selected from the group consisting of: C1-C6 alkyl, halo, —NO₂, —CN, —OH, —NH₂, carbonyl, —CONH₂, —CONR′₂, —CNNR₂, —CSNR₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, —NH(C₁-C₆ alkyl), —N(C1-C6 alkyl)₂, C1-C6 alkoxy, C1-C6 haloalkoxy, hydroxy(C1-C6 alkyl), hydroxy(C1-C6 alkoxy), alkoxy(C₁-C₆ alkyl), alkoxy(C₁-C₆ alkoxy), amino(C1-C6 alkyl), —CONH(C₁-C₆ alkyl), —CON(C1-C6 alkyl)₂, —CO₂H, —CO₂R′, —OCOR′, —OCOR′, —OC(═O)OR′, —OC(═O)NR′, —OC(═S)OR′, —OC(═S)NR′, wherein each R′ is independently selected from hydrogen, alkyl, alkenyl, aryl, heteroaryl a heteroatom, an optionally substituted cycloalkyl, an optionally substituted heterocyclyl, or any combination thereof.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Direct Chemical Modification

As positive charges facilitate intracellular delivery via the endocytosis pathway, several small polyamine derivatives were tested. GFP/IgGs were directly conjugated with either Tetraethylenepentamine (TEPA), triethylenetetramine (TETA) or polyethylenimine (PEI) and the uptake of the protein into the cell was monitored. As PEI was found to be by far superior (data not shown), all further experiments were performed with PEI.

PEIs are long polymers, either linear or branched, with molecular weights usually spanning above 10 KDa, characterized as having the highest cationic charge density among existing polymers. This led to their use as transfection agents, where their high positive charge is used for complexation of negatively charged DNA or RNA and further for internalizing these nucleic acids into cells. While these high molecular weight transfection agents are efficient in in vitro applications, they are less useful in in vivo applications. Thus, ultra-low molecular weight PEI moieties, namely branched PEI molecules of molecular weights ranging from 600 to 1800 Da (as measured my mass spectrometry) were employed. These ultra-low molecular weight PEIs can be chemically and covalently conjugated to a protein payload to be internalized.

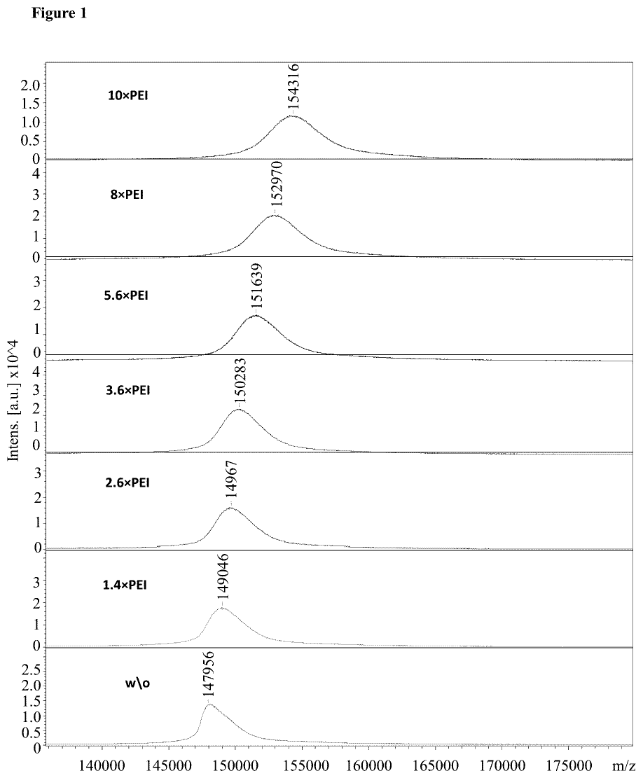

Ultra-low molecular weight PEIs, mainly of 600 Da, were conjugated to IgGs and GFP. As PEI contains many primary amines, conjugation to the protein's carboxylic acid residues of Glutamic and Aspartic acids, as well as to the C-terminal carboxy group, was carried out using carbodiimide conjugation chemistry. Reactions were carried out at an excess of PEI, namely 3500 molar excess, and control of the level of modification was achieved by modulating the level of the carbodiimide agent (N-(3-Dimethylaminopropyl)-N′-ethylcarbodiimide hydrochloride (EDC)) in the reaction. The level of modification was determined by MALDI-ToF mass spectrometry (FIG. 1). In the case of IgG based proteins, the mean level of modification ranged from 1 to about 10 molecules of PEI (600 Da) per IgG molecule, which was achieved with excess levels of EDC ranging from 25 to 400 molar equivalents, respectively. Since the PEI modification is not site selective, it results in a broad distribution of molecular weight moieties, as observed in the MALDI-ToF spectra (FIG. 1). The average modification level was calculated based on the molecular weight value at the measured peak top, minus the molecular weight value at measured peak top of the non-modified protein which gives the weight of added PEI. Dividing this value by the molecular weight of a single PEI molecule used for the modification gives the number of molecules added on average.

Next, PEI-modified and unmodified mouse IgG was incubated with A375 cells for two hours. No additional inducement for transfection/internalization was added. As can be seen in Error! Reference source not found., the non-modified mouse IgG did not enter the A375 cells. In contrast, even low levels of PEI-modification (average of 3 PEI molecules per IgG molecule, X3) enables internalization, while increasing the average level of modification increased the level of IgG observed inside the cells proportionally. Similar results were observed when flow cytometry was used to measure the internalization (data not shown).

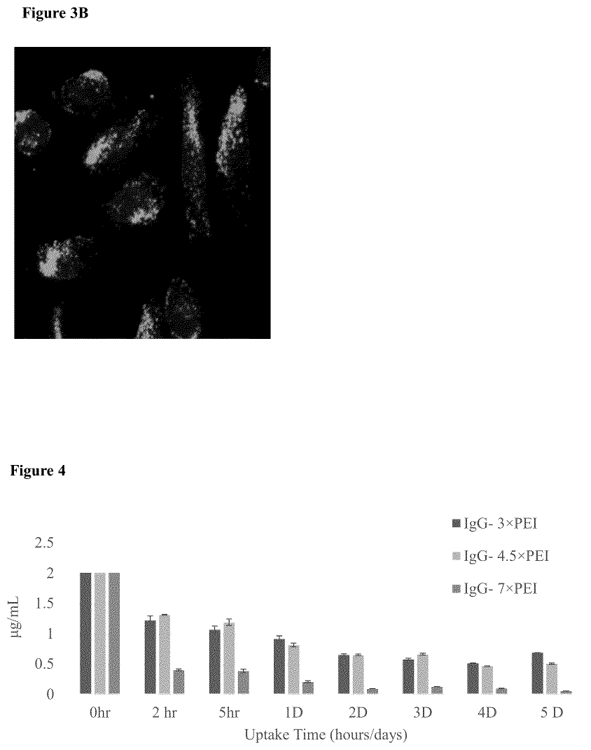

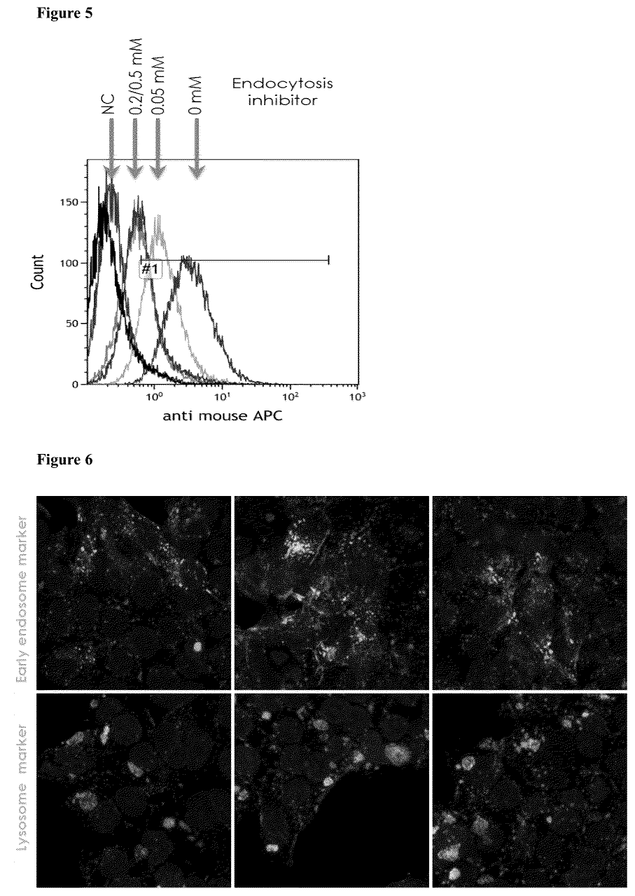

Similar internalization efficiency and dependence on modification level were observed for other payloads, including other IgGs (data not shown) and Green Fluorescence Protein (GFP) (FIG. 3A-3B).

### Example 2: Membrane Crossing Efficiency

Though numerous works with cell penetrating peptides (CPPs) have shown some level of cell internalization, in most cases the efficiency of the initial uptake to the cells was quite low, as high levels of payload in the media were required to produce only a low fraction of molecules actually internalized into the cells. In order to evaluate the efficiency of the initial internalization step, a specific ELISA was used to measure the level of the payload, PEI-modified mouse IgG, in the media as a function of time. As can be seen in FIG. 4, at a high level of PEI-modification (approx. 7) almost all modified IgG is internalized (95%) and most of the internalization occurs during the initial 24 hours of incubation (grey bars). Even at lower levels of modifications (approx. 4.5 and 3), the internalization step is quite efficient, exhibiting an overall uptake of about 75% of the total payload after 5 days of incubation (FIG. 4, blue and orange bars) and follows similar kinetics as the highly modified IgG. The incomplete and slower uptake is likely related to IgGs that are much less modified than the average modification level. The PEI-modification results in a somewhat broad distribution of modified moieties (see FIG. 1) and those molecules that are unmodified or lowly modified (less than 3) are likely still present in the media even after 5 days. This cell-membrane crossing efficiency was observed at various initial media concentrations, ranging from 0.2 to 40 μg/mL of IgG.

### Example 3: Endocytosis Pathway Exploited by PEI-Modified Proteins

Cationized moieties are thought to internalize using a natural endocytosis mechanism. The two relevant endocytosis mechanisms are caveolin- and clathrin-mediated endocytosis. The exact mechanism of internalization was elucidated by performing the internalization of PEI-modified IgG in the presence of known specific endocytosis inhibitors. A375 cells were incubated overnight with PEI-modified mouse IgG (4.5 PEIs) in the presence of Clathrin inhibitors (Amantadine or Chlorpromazine) or a Caveolin inhibitor (Genistein). Genistein, a known Caveolin inhibitor, indeed inhibited the internalization of PEI-modified IgG (Error! Reference source not found.). Quite interestingly, addition of Chlorpromazine, a known Clathrin inhibitor, resulted in increased internalization. This corroborates that Caveolin-mediated endocytosis is exploited by the PEI-modified payloads for internalization as inhibition of Clathrin-mediated endocytosis caused a compensatory increase in Caveolin-mediated endocytosis in the cells.

### Example 4: Endosomal Escape

As described above, perhaps the most challenging hurdle in intracellular delivery of biologic therapeutics is their endosomal escape in order to avoid catabolism of the therapeutic. In order to evaluate whether PEI-modified proteins can escape from endosomes, their internalization was followed using confocal microscopy and counter staining of endosomal and lysosomal markers. PEI-modified IgGs were incubated for 5 hours with HEK293 cells expressing fluorescent endosomal/lysosomal markers. The cells were analyzed by confocal microscopy. Endosomes and lysosomes were transfected with Green fluorescent markers (Cell light Endosome, Molecular Probes, Cat. No. C10586 or Cell light Lysosome, Molecular Probes, Cat. No. C10596) while IgGs were stained with Red-fluorescent anti-mouse antibody. As can be seen in Error! Reference source not found., no major co-localization between the internalized IgG and the early endosomal or lysosomal vesicles was observed. This demonstrated that indeed the PEI-modified payload has successfully escaped the endosomes. These experiments were repeated with the endosomal/lysosomal markers EEA1, transferrin and Calcein, and the same results were observed (data not shown). It must be noted that co-localization is not observed even with early endosomes, suggesting that endosomal escape of PEI-modified proteins occurs rapidly, at the very beginning of the endosomal pathway.

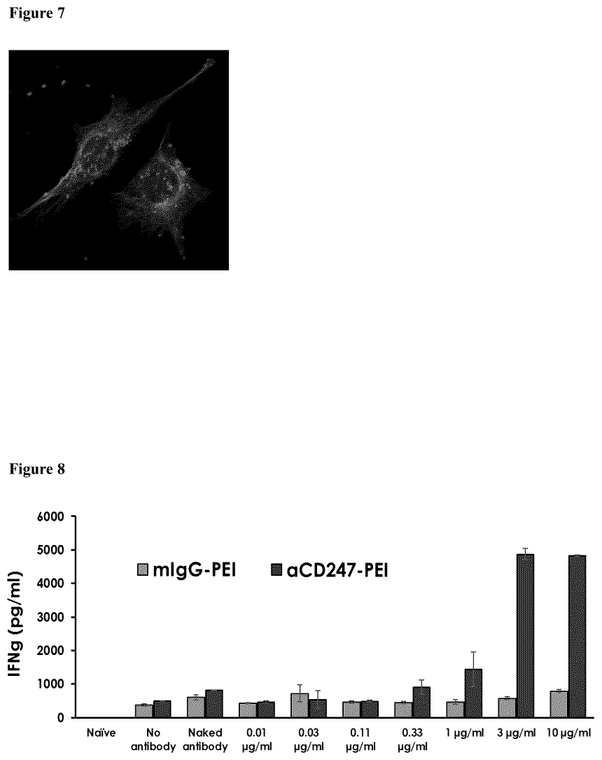

While no co-localization is observed between the PEI-modified IgGs and various endosomal and lysosomal markers, the staining of these IgGs appear punctate in nature. A punctate profile may suggest the inclusion of the internalized IgGs in some sort of vesicles. In order to further corroborate the endosomal escape of PEI-modified proteins, the proteins were labeled with a pH sensitive fluorescent dye, 5(6)-carboxynaphthofluorescein. This dye only fluoresces at pH values above 7. All vesicles of the endosomal pathways are acidic, as the pH of early endosomes is already below 7 and only decreases as the endosomes mature into lysosomes. PEI-modified, pH-sensitive labeled IgGs were incubated with HeLa cells and the cells were analyzed by confocal microscopy. The cells clearly show the unique Red fluorescence of the pH-sensitive dye, indicating the PEI-modified proteins are in a non-acidic compartment (FIG. 7), such as in the cytoplasm. This is further evidence that PEI-modified proteins do efficiently escape the endosomal pathway.

Finally, a functional assay was employed to validate that PEI-modified proteins efficiently escape the endosomes following their internalization. To this end, a functional monoclonal antibody against CD247, also known as CD3-zeta chain, was modified with PEI and internalized into CD3+ primary cells. The intracellular domain of the CD3 receptor is responsible for T cells activation through its Immunoreceptor Tyrosine-based Activation Motifs (ITAM). The chosen monoclonal anti-CD3 zeta chain antibody (Sigma, Clone ZT-10, Cat. No. SAB4200446) binds one of the ITAMs in the CD3 zeta chain.

Internalization of the PEI-modified anti-CD247 and a PEI-modified mouse IgG as negative control was carried out into CD3+ cells that were pre-stimulated with beads coated with anti-CD3/CD28 antibodies. The media was monitored for Interferon gamma (IFNγ) secretion as a marker for changes in the cells' activation level. Although a full antibody would not be expectable to be stable in the cytoplasm due to the presence of structurally essential disulfide bonds, the binding of this antibody would activate a signaling cascade that would culminate in IFNγ secretion. Thus, even a small amount of initial activation would produce a quantifiable increase in cytokine levels. As such, even with its expected cytoplasmic instability, internalization of the antibody would be expected to produce an observable IFNγ response.

Internalization of the modified antibodies to the CD3+ cells was verified by intracellular FACS analysis (data not shown). Internalization of the anti-CD247 PEI-modified mAb (4.5×PEI) resulted in a dose dependent activation of the CD3 cells as measured by elevation of IFNγ secretion (FIG. 8). PEI-modified mouse IgG did not cause any CD3 cell activation at the range of antibody concentrations used in this experiment. This result further corroborates endosomal escape of the PEI-modified proteins and their ability to exert biological and clinical effects in target cells.

### Example 5: Cytosolic Dispersibility and Payload-Carrier Solution

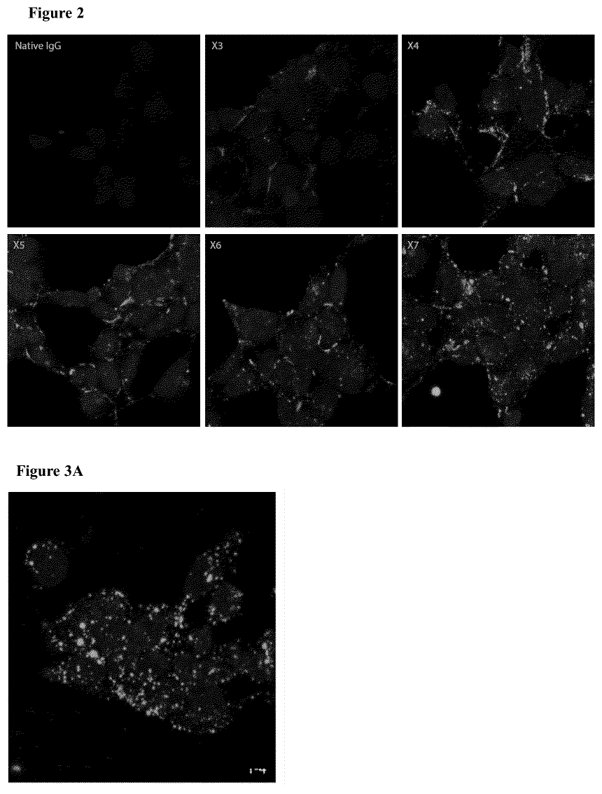

Although endosomal escape of the PEI-modified proteins clearly occurs, microscopy images of various internalized proteins exhibit a punctate profile (FIGS. 2, 3, 6 and 7). It was hypothesized that while the PEI modification is highly efficient in cell membrane crossing and endosomal escape, the same modification impedes the PEI-modified proteins from dispersing efficiently in the cytoplasm. This dispersibility problem may be the result of a strong electrostatic interaction between the strongly cationized internalized protein and various cytoplasmic proteins, mainly cytoskeletal proteins, most of which are negatively charged. Low cytoplasmic dispersibility will negatively affect the efficacy of any intracellular biologic agent as the agent may not be able to reach its intracellular target, whether in the cytoplasm or in other intracellular compartments or organelles.

In order to overcome this intracellular dispersibility hurdle, disulfide bonds were incorporated into a linker between the therapeutic agent and a universal carrier pre-modified with PEI. The linkage selected was a chemical linker, with a disulfide bond incorporated. This labile disulfide bond will be cleaved upon endosomal escape due to the high reducing potential of the cytoplasm, mainly due to its high concentration of glutathione, thus releasing the therapeutic agent from its cationized carrier and enabling the free movement of the therapeutic agent in the cell's cytoplasm. Use of a carrier also has the advantage of removing the risk that the PEI could be conjugated at a functional location, and/or that it would inhibit the function of the therapeutic. By using a selective conjugation chemistry for the linker to the therapeutic agent at a specific site or domain, one can control the exact location of linker conjugation, diverting it away from the active domains of the therapeutic agent.

A schematic representation of this carrier and payload methodology is depicted in FIG. 9. As can be seen, the carrier protein bears the PEI groups and is responsible for membrane crossing and endosomal escape. These groups can be conjugated to the carrier protein either randomly or site-selectively. In the case of random conjugation, the level of conjugation can be controlled by choosing specific reaction conditions as described hereinabove.

The carrier protein itself is preferably selected from a list of human endogenous proteins to avoid immunogenicity issues. Further, a protein which is commonly found in the blood and which has a naturally long circulatory half-life is preferable. Lastly, a protein that is able to deliver its payload to areas in the body where the therapeutic agent is desired would be advantageous. Examples for such areas in the body can include tumors and their microenvironment, as well as sites of inflammation that are important for autoimmune disease and many other pathological conditions. Human Serum Albumin (HSA) was therefore selected. HSA is a circulatory protein, with a long half-life and has been shown to traffic to tumors and sites of inflammation, and even to deliver payload to those sites, though only to the extracellular milieu (Liu et al. BMC Biotechnology 2012, 12:68; Kratz, F., Journal of Controlled Release 132 (2008) 171-183; Um et al., Bioconjugate Chem., 2019, 10.1021/acs.bioconjchem.9b00760, Wunder A. et al., J Immunol 2003; 170:4793-4801; Yazaki P. J. et al., Nuclear Medicine and Biology, 35 (2008) 151-158).

The therapeutic agent is conjugated to a single linker at a single conjugation site along the polypeptide chain of the biologic agent. This conjugation site is located in a position where it will not interference with the agent's activity. The linker can be selected from a peptide linker, a chemical linker or a combination. A polymer-based linker, PEG, was selected. As discussed above, a labile bond, sensitive to the specific conditions in a cell's cytoplasm, is also incorporated.

In order to evaluate the efficiency of the carrier-payload methodology, GFP was conjugated to a PEI-cationized HSA via a PEG-based linker incorporating a disulfide bond. eGFP (Biorbyt, Cat. No. orb84840) was modified with NHS-PEG₄-SPDP. PEI-modified HSA (11×PEI) was also further reacted with NHS-PEG4-SPDP following reduction of this SPDP to a free thiol using DTT. The SPDP-activated eGFP was reacted with the HSA-PEI-free thiol to create the GFP-HSA conjugate with the labile disulfide bond incorporated in the linker.

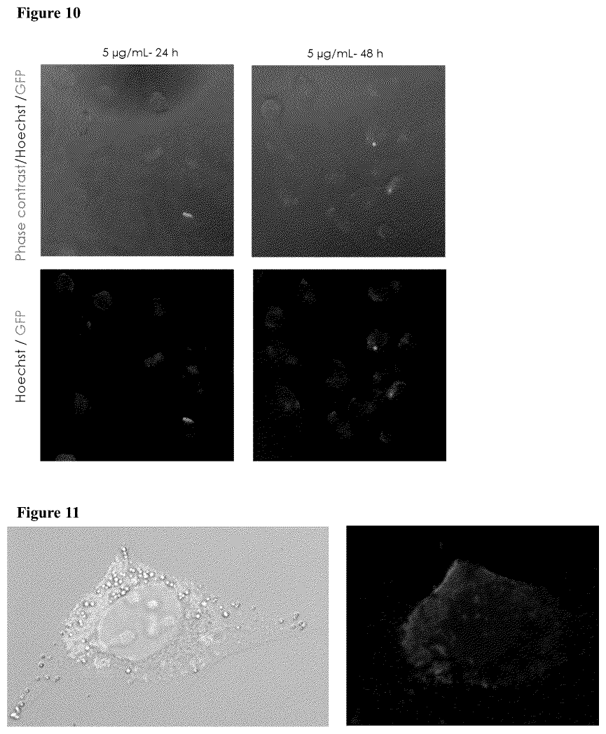

As opposed to the punctate profile of internalized PEI-modified GFP (FIG. 3), when the GFP disulfide-linked to PEI-cationized HSA was internalized to cells it yielded a fully dispersed profile without puncta (FIG. 10). To verify the efficient dispersal of the internalized therapeutic payload, a full therapeutic anti-TNFα monoclonal antibody (Humira®) was linked to PEI-modified HSA via a PEG-based linker incorporating a disulfide bond. Similar to GFP, the antibody was modified with NHS-PEG₄-SPDP. PEI-modified HSA (11×PEI) was also further reacted with NHS-PEG₄-SPDP following reduction of the SPDP to a free thiol using DTT. The SPDP-activated antibody was reacted with the HSA-PEI-free thiol to create the antibody-HSA conjugate with the labile disulfide bond incorporated in the linker. As can be seen in FIG. 11, the internalized monoclonal antibody disperses throughout the cytoplasm, again corroborating the hypothesis that direct cationization impeded free dispersion in the cytoplasm and demonstrating the effectiveness of the solution of separating the payload from its cationized carrier.

### Example 6: Cytosolic Stability

The therapeutic agents need to not only enter the cytoplasm of target cells but also exert their biological activity. Even molecules that are transported to other subcellular locations (nucleus, ER, mitochondria, etc.), still would pass through the cytoplasm. Many biological therapeutic agents are based on an antibody scaffold or its derivatives. Often these therapeutic agents bind a specific target and either antagonize or agonize that target. The binding activity of these agents is totally dependent on their tertiary and quaternary structures. In antibody-based molecules, either full IgGs or their truncated derivatives (Fabs, scFvs, etc. . . . ), these structures are based on, and stabilized by, disulfide bonds, either intra-or inter-chain. However, as mentioned above, the cytoplasm, as well as other subcellular organelles and compartments, is characterized by a highly reducing environment. The major reducing agent, Glutathione, has a cytosolic concentration ranging from 1 to 11 mM. In contrast, its plasma level is in the low micromolar values. This characteristic of the cytoplasm has hampered the use of antibody-based agents, as well as other biological agents which utilize disulfide bonds in their structure, as an efficient intracellular route of treatment.

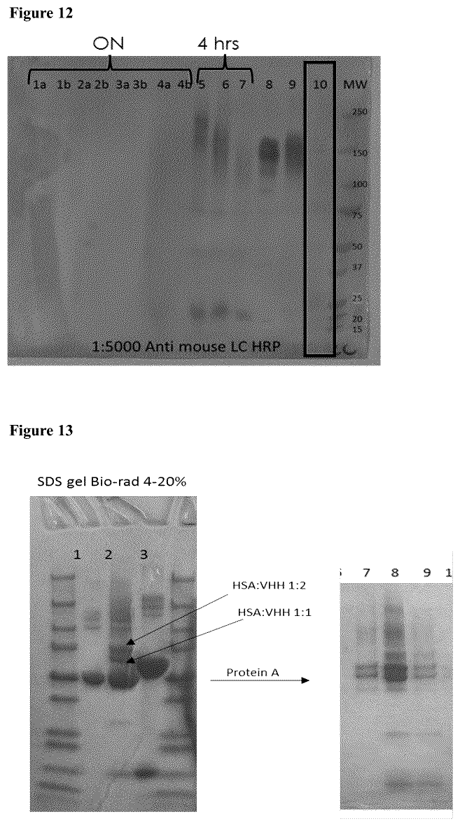

An in vitro experiment showed that exposure of an antibody to cytosolic levels of Glutathione (GSH) leads to the reduction of disulfide bonds after only a few hours, as seen by the appearance of multiple bands on an SDS-PAGE Western blot as detected by an anti-light chain antibody (lanes 5-7, FIG. 12). Overnight exposure leads to lack of detection of the antibody in Western blot (lanes 1-4, FIG. 12), probably due to loss of 3D structure or even aggregation and sedimentation. The latter are known phenomena of intracellularly expressed antibodies and their derivatives (Kabayama, H. et al., Nature Communications 2020, (11), 336). Any such effect on an intracellular biological agent would be detrimental to its ability to bind a biological target and to exert a therapeutic effect.

In light of these intracellular stability issues, single-domain antibodies were selected as the therapeutic payload. Single-domain antibodies are single-chain protein-based molecules with the ability to bind other proteins. Their structure lacks any essential disulfide-bonds, making them resistant to the cytosol's reducing environment. Such single-domain binding proteins include truncated forms of heavy chain antibodies (HcAbs), either Camelid-based variable heavy homodimers (VHH), also known as nanobodies, or Shark-based immunoglobulin novel antigen receptors (IgNAR). Other examples of such single-domain binding proteins include the designed ankyrin repeat proteins (DARPins), and genetically engineered antibody mimetic proteins.

Single domain binding proteins, and VHHs in particular, are well suited as payloads. Their lack of structurally-essential disulfide-bonds makes them resistant to the cytoplasmic conditions. They are extremely small, about 15 KDa for VHHs or 20 KDa for DARPins, a fact that assists in their cytosolic dispersibility. Their single domain characteristic dictates that they would not cause any accidental intracellular cross-linking effects. They are easily engineered to include 2 or more moieties in different architectures to enable more complex binding profiles. They are not considered immunogenic and have a good safety profile. A major feature is their compatibility with site-selective conjugation to the carrier. For example, in both VHHs and DARPins, their C-terminus is located away from their antigen binding regions (CDRs), enabling the utilization of this site for conjugation without affecting antigen-binding. Further, the C-terminus can be easily engineered to include a single cysteine amino acid with a free sulfhydryl group for conjugation. This group can be conjugated to a carrier equipped with a thiol-reactive group. The C-terminal free sulfhydryl can be conjugated directly to the carrier or alternatively via a linker.

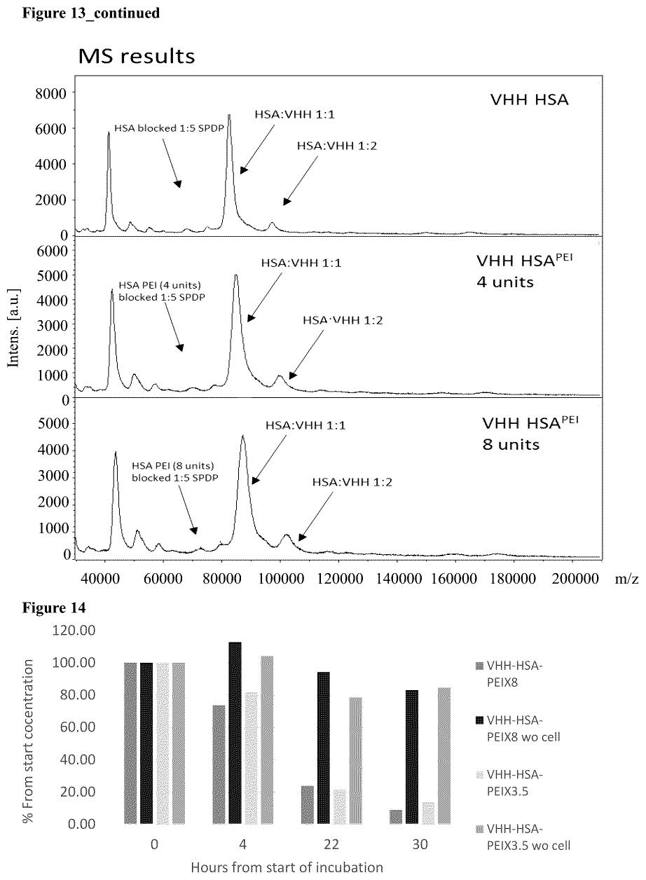

A commercial anti-vimentin VHH (QVQ, Q60c) comprising a single cysteine amino acid in its C-terminal was conjugated to a PEI-modified HSA carrier further modified by thiol reactive groups. The PEI-modified HSA was coupled to NHS-activated PEG linkers carrying a 2-pyridyldithio group (NHS-PEG.-SPDP, n=4). The SPDP readily reacts with the free-thiol of the VHH generating the VHH-HSA moiety, where the connection between the VHH payload and the HSA carrier includes a disulfide bond. FIG. 13 presents the profiles of the reaction mixture before and after purification, as well as the ability of the bond between the carrier and the payload to be cleaved under reducing conditions. The conjugation to the VHH was similar regardless of the level of PEI. Additionally, in all reactions, the payload was efficiently cleaved following treatment with 5 mM of GSH, mimicking cytosolic reducing conditions.

The cell internalization efficiency of the anti-vimentin-PEI-modified HSA conjugate was evaluated by the disappearance of the conjugate from the culture media upon incubation of A375 cells with the conjugate. The VHH was conjugated to HSA modified with PEI at two levels, an average of 3.5 PEI molecules and 8 PEI molecules per HSA. As already seen for PEI-modified IgGs (FIG. 4), the VHH-carrier conjugate was efficiently internalized by the cells (FIG. 14), with almost 80% of the conjugate internalized during the first 24 hours of incubation. The level of conjugate was measured using an in-house developed two-sided ELISA measuring only VHHs conjugated to HSA, utilizing an anti-VHH antibody as capture and an anti-HSA antibody for detection. Surprisingly, both PEI-modification levels exhibited very similar internalization efficiency and kinetics, with only a very slight advantage to the 8-PEI modification. The level of these VHH-HSA conjugates in cell media without cells was evaluated in order to make sure that the observed reduction in their level is not due to degradation. Only a slight drop was observed when the cells were absent (FIG. 14), suggestion that indeed the dramatic reductions in VHH levels in the presence of cells was due to VHH internalization.

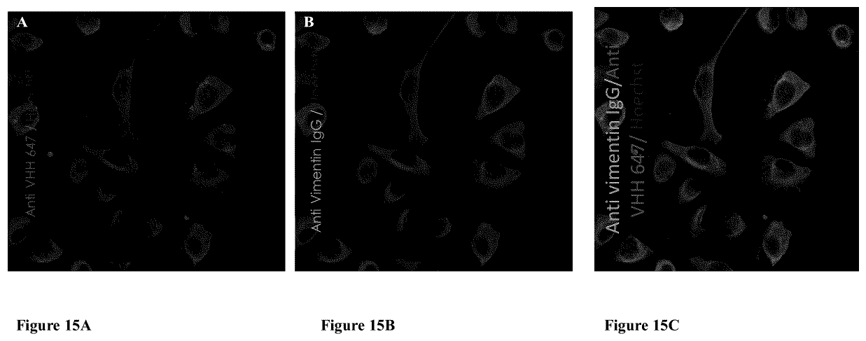

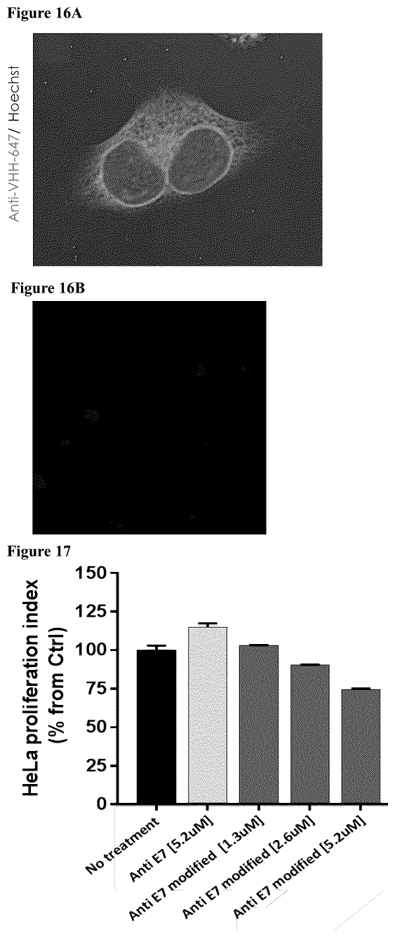

In order to further evaluate the efficiency of endosomal escape, as well as the dispersibility of the payload VHH in the cytoplasm, the anti-Vimentin-VHH conjugated to PEI-modified HSA (average of 11 PEIs per HSA molecule) was incubated with A375 cells for 24 hours and the cells were analyzed by confocal microscopy (FIG. 15A-15C). An anti-VHH antibody conjugated to AlexaFluor 647 was used to show the presence of the VHH inside the cells in a defuse profile (FIG. 15A). The cells were co-stained for vimentin using a standard fluorescently labeled anti-vimentin antibody (FIG. 15B). Careful examination of the two images clearly shows that the profile obtained by the anti-VHH staining is practically identical to the vimentin staining, suggesting that the anti-vimentin VHH was successfully delivered to the cells' cytoplasm where it was released from its carrier and was able to find and bind its target (FIG. 15C). Further evidence to this binding can be seen in FIG. 16A, where a close up of one of the cells exposed to the anti-vimentin VHH conjugated to the PEI-modified HSA clearly shows a vimentin cytoskeleton pattern as visualized by anti-VHH antibody staining. FIG. 16B shows that cells exposed to just the anti-vimentin VHH, with no carrier conjugation, exhibit no intracellular VHH staining.

The binding of the internalized anti-vimentin VHH to its vimentin target (FIG. 15A, FIG. 16A) also demonstrates that the VHH agent has maintained its structural stability inside the cytoplasm. The ability to bind to targets, as is the case with all binding biological agents, is crucially dependent on its structure and the stability of this structure. This data therefore supports the choice of single domain binding proteins as the payload agents of the invention.

Another important characteristic of the presented delivery system is the efficiency and uniformity of internalization, in that all cells in the media exhibit internalization. This can be seen throughout the different microscopy images, including the images in FIG. 15A-15C in particular. Many attempts at intracellular delivery of proteins known in the literature exhibit results where only part of the cells exhibit protein internalization, suggesting very low efficiency.

### Example 7: In Vitro Functional PoCs

A further assessment of the functionality of intracellular delivered biologics, was performed using a VHH against the E7 protein of Human Papilloma Virus (HPV). Nearly all cervical cancers are associated with human papillomaviruses (HPV) infection, with two types, HPV16 and HPV18, accounting for 70% of cases. One of the primary oncoproteins of HPV is the E7 protein. E7 induces and maintains the malignant phenotype through its interaction with the retinoblastoma protein (RB1). E7 disrupts the function of host RB1 protein leading to stimulation of uncontrolled cell proliferation. E7 can also interfere with host histone deacetylation mediated by HDAC1 and HDAC2, leading to transcription activation. Prior research suggested that inhibition of E7 function inhibits the growth of HPV-positive cervical cancer cells. Li et al (Molecular Immunology, 2019, 109, 12-19) showed that transfection of a plasmid encoding a VHH against the E7 protein in HPV positive cells, which was used due to lack of an efficient intracellular delivery system for the protein itself, can interfere with E7 activity (disrupting the E7-RB1 interaction), leading to a reduction in the proliferation of HPV positive cells. The inventors have expressed and purified the same anti-E7 VHH with the addition of a C-terminal cysteine and conjugated it to the intracellular carrier of the invention. HPV positive HeLa cells were incubated with the unmodified anti-E7 VHH or with the VHH conjugated by a cleavable linker to PEI-modified HSA carrying an average of 3.5 PEI molecules per HSA molecule. Following incubation, the viability of the cells was measured using a standard MTT viability assay. While the unmodified VHH had no effect on cell proliferation, the cells incubated with the VHH conjugated to the PEI-modified carrier exhibited a dose-dependent reduction in cell viability, demonstrating that the anti-E7 VHH was successfully internalized and was able to inhibit the effect of E7 in the HeLa cells (FIG. 17).

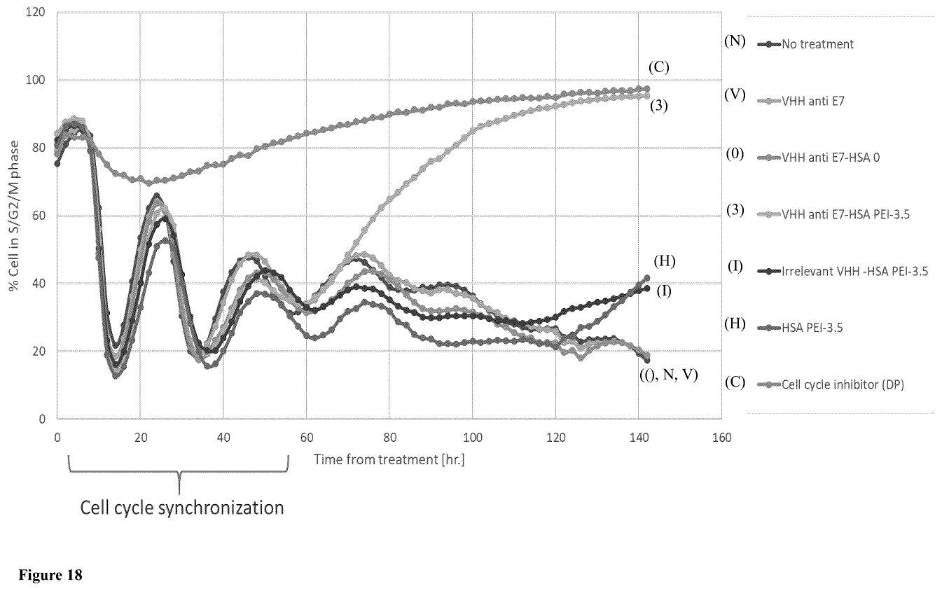

In order to ascertain the amplitude of the intracellular effect of E7 inhibition in HPV-positive cell lines, live cell analysis systems, such as Incucyte®, were employed. As E7 affects cell cycle control, a specific HPV-positive HeLa cell line was used, termed Fluorescent Ubiquitination-based Cell Cycle Indicator (FUCCI). The FUCCI reporter system allows the following of the different cell cycle stages of the cell. Cells in G1 fluoresce in Red and cells in S or G2 or M fluoresce in Green. Cells were synchronized with Thymidine for 24 hours prior to introduction of the different treatments into the cells' media. The cells were treated with the anti-E7 VHH conjugated by a cleavable linker to the PEI modified HSA and with the following controls: no treatment, the modified HSA, the modified HSA conjugated to an irrelevant VHH (anti-Vimentin), the unmodified anti-E7 VHH, anti-E7 VHH conjugated to unmodified HSA and a cell cycle inhibitor (DP, CDK4/6 inhibitor). As can be seen in FIG. 18, while all the controls have no effect on the cell cycle which remains similar to the non-treated cells, the anti-E7 VHH conjugated to the PEI-modified HSA (3.5 PEIs) had a dramatic effect, leading to a cell cycle arrest similar to the use of the direct cell cycle inhibitor. As no commercial E7 inhibitor exists, a CDK4/6 inhibitor, Palbociclib isethionate (PD0332991, Sigma, Cat. No. PZ0199), known to cause cell cycle arrest, was used as positive control. The anti-E7 VHH conjugated to modified HSA led to a similar effect as the inhibitor, only at different kinetics. Further, as can be seen in FIG. 19, this arrest led to the death of the HPV-positive cells.

Both the anti-vimentin and anti-E7 VHHs have shown their durability in the cytoplasm's reducing environment. In order to further exemplify the compatibility of single domain binding proteins, that do not rely on disulfide bonds to stabilize and maintain their structure, to the current invention, the inventors have used a designed ankyrin repeat protein (DARPin) as a binding agent. DARPins are genetically engineered antibody mimetic proteins typically exhibiting highly specific and high-affinity target protein binding. They are derived from natural ankyrin proteins, one of the most common classes of binding proteins in nature, which are responsible for diverse functions such as cell signaling, regulation and structural integrity of the cell. DARPins consist of several repeat motifs and their molecular mass is about 14 to 18 KDa (kilodaltons). They are also characterized by not being dependent on disulfide bonds for their structural integrity, similarly to VHHs.

To this end, a DARPin against K-RAS was selected. RAS proteins play key roles in signal transduction as molecular switches. RAS is the most important target in cell transformation, involved in cell proliferation and differentiation through the RAF-MEK-ERK cascade and cell survival through activation of PI3K. Mutations of the RAS proteins (K-, H- or N-RAS) create constitutively activated GTP-bound forms that promote cell transformation in a signal-independent manner. Activating RAS gene mutations are found in as many as 30% of cancers in humans, with the highest frequencies in pancreas, colon and lung adenocarcinoma. Oncogenic RAS has been shown to be essential for early onset of tumors and necessary for maintenance of tumor viability. The most central position of RAS mutations is in Glycine 12, such as G12D and G12V.

Guillard et al. (Guillard, S. et al. Nat. Commun. 2017, 8, 16111) have generated an antibody mimetic, DARPin K27, which inhibits nucleotide exchange of Ras. K27 binds preferentially to the inactive Ras GDP form with a Kd of 4 nM and structural studies support its selectivity for inactive Ras. Intracellular expression of K27 by transfection of a DARPin encoding vector was shown to significantly reduce the amount of active Ras, to inhibit downstream signaling, in particular the levels of phosphorylated ERK, and to slow the growth in soft agar of HCT116 cells. This group states that a “ . . . barrier arises from the fact that Ras is intracellular. DARPin K27 has no intrinsic ability to enter cells and therefore cannot access Ras when the DARPin is added extracellularly. Although there have been reports of delivery of DARPins to the cytoplasm of the cell . . . substantial increases in efficiency would be required to make the approach viable therapeutically. It may be challenging to develop a small molecule inhibitor binding at the same site as DARPin K27, since the scaffold binds across a broad surface, rather than defining a pocket.”

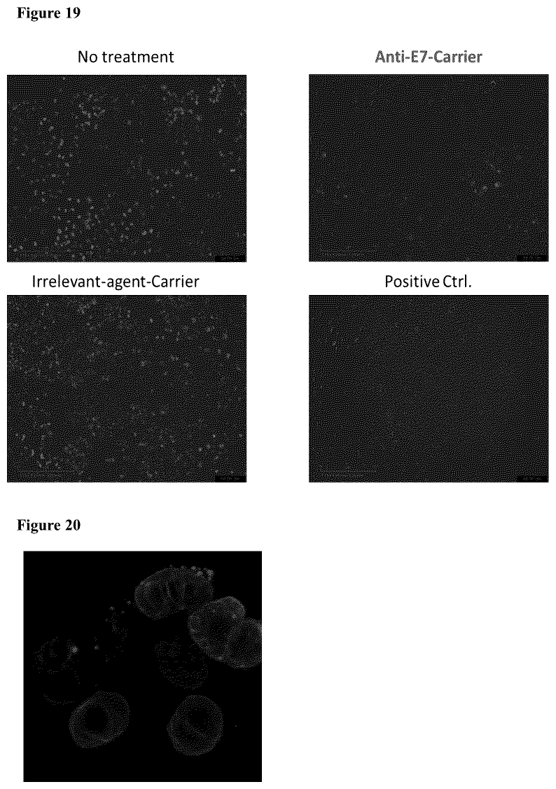

DARPin K27 was expressed based on the published sequence, and was conjugated to an HSA carrier, modified with an average of 3.5 PEI molecules. Internalization and KRAS binding were evaluated by confocal microscopy (FIG. 20). The K27 DARPin was not dispersed in the cytoplasm but rather localized to the inner side of the plasma membrane, the major location of KRAS. Interestingly, some cells exhibited both a punctate profile and an inner membrane localization in regard to the internalized DARPin. The puncta may be a result of DARPin that still has not escaped the endosomes or did not yet separate from the carrier to freely find its target.

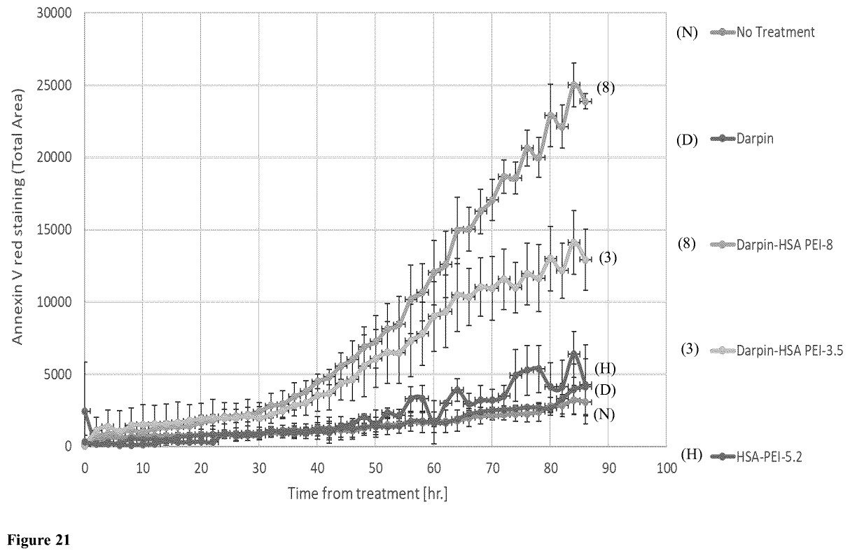

In a further test, pancreatic ductal carcinoma cells (SU8686) were incubated with the anti-KRAS DARPin K27 which was conjugated to the HSA carrier. The carrier was modified with either 8 or 3.5 PEI molecules. The apoptotic state of the cells was monitored using the classical Annexin V assay and was visualized using the Incucyte® continuous cell monitoring system. Cells exposed to the DARPin conjugated via a cleavable linker to the PEI modified HSA exhibit a dramatic elevation in apoptosis (FIG. 21), especially for the DARPin conjugated to the carrier with the high level of PEI modification. This is likely due to enhanced internalization and quicker kinetics. In contrast, cells that were untreated, treated with unmodified DARPin or treated with the PEI-modified HSA (8 molecule) alone exhibited baseline levels of apoptosis emphasizing the intracellular effect of the DARPin.

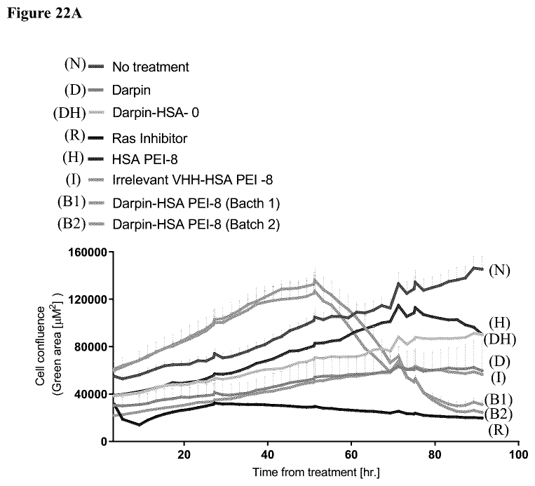

Guillard et al. have shown that the anti-KRAS DARPin K27 binds to native, non-mutant inactive KRAS and to KRAS with different mutations, mostly in the G12 position. Thus, anti-KRAS DARPin K27 was evaluated for its intracellular effect on proliferation and apoptosis of a HeLa cell line. This cell line is characterized by a constitutive expression of GFP in its nuclei, enabling easy following of these cells using continuous image monitoring methods, such as Incucyte®. Two different preparations of the anti-KRAS DARPin were conjugated to HSA carrying 8 PEI molecules and its effect on the HeLa-GFP cells was compared to the following control treatments: no treatment as negative control, treatment with a Pan-Ras inhibitor as positive control, treatment with the unmodified DARPin, treatment with the DARPin conjugated to non-modified HSA, HSA carrier modified with 8 PEI molecules, and an anti-Vimentin VHH conjugated to HSA modified with 8 PEI molecules. As can be seen in FIGS. 22A-22B, both preparations of the anti-KRAS DARPin conjugated to HSA carrying 8 PEI molecules dramatically affected both the proliferation (FIG. 22A) and apoptosis (FIG. 22B) of the HeLa-GFP cells. None of the control treatments had any effect on these parameters. The effect on apoptosis can also be seen in FIG. 23, where the red staining of cells (apoptotic cells) treated with the anti-KRAS DARPin conjugated to PEI-modified HSA and the loss of GFP as the cells die is clearly observed.

### Example 8: Pharmacokinetics and Biodistribution (PK & BD)

The PEI modification confers a concentrated and strong positive charge on the carrier protein. In contrast, plasma components and cellular membranes generally are negatively charged. Positively charged proteins are known to be “sticky” due to electrostatic bonds with these negatively charged components. This “stickiness” can lead to short half-lives and to issues with biodistribution. This phenomenon is known in proteins which are naturally positively charged and characterized by somewhat basic isoelectric points (pI). The pharmacokinetic and biodistribution profiles of a PEI modified protein may be affected by its strong positive charge. Furthermore, such adherence can also lead to “trapping” of the administered positively charged protein in the injection site. In order to avoid, or at least minimize, these effects the effect of the level of PEI modification was examined.

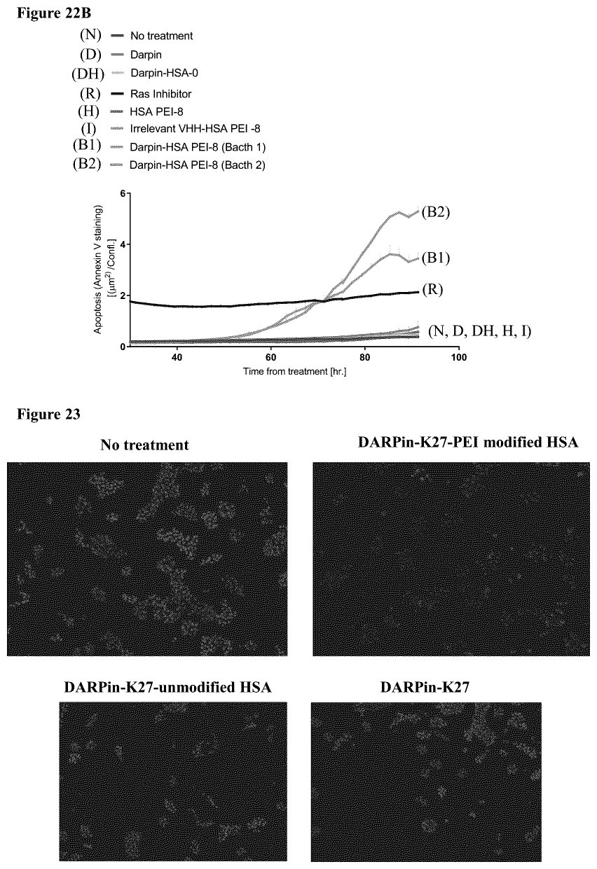

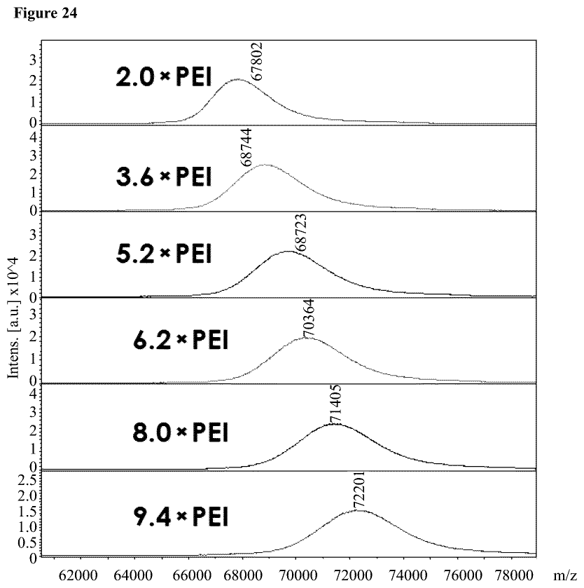

By identifying and utilizing the lowest level of PEI modification that is still effective in crossing the cell membrane and in enabling endosomal escape the PK and biodistribution of the proteins of the invention can be improved. In order to identify this modification level, HSA carriers were produced with the following average levels of PEI modifications: 2.0, 3.6, 5.2, 6.2, 8.0 and 9.4. These levels were ascertained by the analysis of the resulting modified HSAs using MALDI-ToF mass spectrometry (FIG. 24). These carriers were further conjugated to the anti-Vimentin VHH and the internalization of these conjugates was evaluated using confocal microscopy (FIG. 25). As can be seen in the confocal microscopy results, even VHH conjugated to HSA with 2.0 and 3.6 PEI molecules exhibits a clear presence inside the cells. While the use of a carrier modified with higher levels of PEI seems to lead to apparent higher levels of VHH inside the cells, the profile of the internalized VHH in those cases is more punctate and less dispersed, probably due to slower detachment of the VHH from the carrier due to the higher positive charge. The stronger staining of VHH inside the cells in the case of HSA modified with more than 3.6 PEIs/HSA may be the result of stronger imaging due to the concentrated punctate profile and may not necessarily point to absolute higher levels.

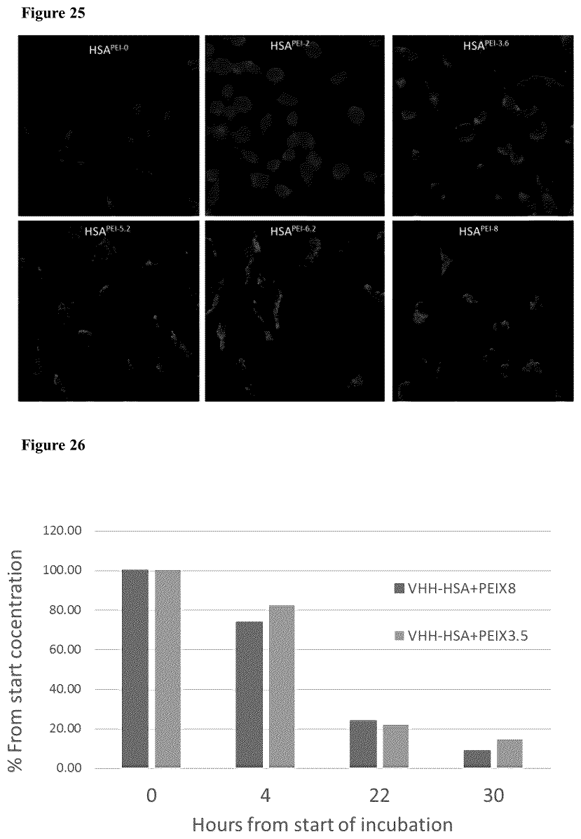

The results of the confocal microscopy were also correlated with the internalization levels of these conjugates by measuring the residual levels of the conjugates in the cells' media using a specific ELISA (FIG. 26). Unlike the results observed with IgGs directly modified with PEI (FIG. 4), the level and kinetics of the HSA carrier (with VHH conjugated) are less affected by the level of modification. With both 3.5 and 8 molecules of PEI the internalization is efficient, and its magnitude and rate are very similar. These results suggest that indeed the HSA to be used as carrier can be modified with just 3.5, or even fewer, PEI molecules, a fact that will reduce the stickiness of the HSA carrier in circulation.

### Example 9: Charge Masking (Non-Covalent)

Non-covalent charge masking can be accomplished by neutralizing the positive surface charges of PEI, such as by electrostatic bonding of negatively charged molecules to the PEI. Specifically, the inventors postulated that molecules with multiple negative charges such as phytic acid, may interact electrostatically with multiple positive charges of PEI to establish an efficient charge masking in-vivo.

To evaluate the effect of charge masking on the pharmacokinetics and biodistribution of PEI-modified HSA, HSA-PEI×8 was treated with 20 mM of phytic acid, dialyzed to remove excess phytic acid and intravenously injected to mice. Non-treated HSA-PEI×8 was administered as control. Concentrations of HSA were measured in the plasma of the Balb-c mice using an in-house ELISA and the results are presented as percent from the injected dose. The inventors observed that the HSA-PEI×8 clears quickly from the circulation as evident by the fact that even at 5 minutes post administration only 35% of the injected dose can be quantitated. At 30 minutes, 95% of the HSA-PEI×8 cannot be located in the plasma of the treated mice. The phytic acid masked HSA-PEI×8 however behaves quite differently from the non-masked HSA-PEI×8 although the masking has led to even faster clearance from the circulation.

In order to better understand these effects, a Near-IR fluorescently labelled (VivoTag 750-S, PerkinElmer) version of the above test particles was prepared and used in a PK experiment utilizing an IVIS imaging system (PerkinElmer). Athymic nude mice were administered with 2 levels of HSA-PEI×8 (125 and 250 nmol/Kg) and HSA-PEI×8 masked with phytic acid (5 mM) which was administered at 125 nmol/Kg. The animals were imaged in the IVIS system at different time points (FIG. 27A) up to 48 hours post-administration.

While it seems that the fluorescent labeling has affected the PK and biodistribution of the labelled proteins (data not shown) the comparison of the phytic acid masked HSA-PEI×8 to the non-masked protein clearly shows the dramatic effect of masking. While the HSA-PEI×8 rapidly concentrates in the kidneys and liver of the animals, the phytic acid masked HSA-PEI×8 avoids this fate. These effects are also evident when imaging the different organs of the animals harvested 48 hours post injection (FIG. 27B).

Based on these results as well as other results, it is the understanding of the inventors that while the transiency effect of non-covalent phytic acid masking in in vitro settings was quite satisfactory (results not shown), in in vivo settings the masking effect of phytic acid is lost quite rapidly. It is postulated, that the inability of phytic acid to establish an efficient charge masking in vivo, is probably due to fierce competition by various divalent metal ions, such as calcium and magnesium, which have high affinity towards the phytic acid. Additionally, it should be noted that while Zeta potential measurements have shown that the phytic acid dramatically reduces the positive charge of PEI-modified HSA, the positive charge is basically neutralized in a way that the surface charge of the masked HSA-PEI is close to zero. Proteins with surface charge around zero are known to be unstable in solution and additionally zero-charged proteins are not known to have efficient pharmacokinetic profiles.

These insights and drawbacks of the non-covalent masking have led the inventors to seek masking solutions based on covalent masking.

### Example 10: Charge Masking (Via Covalently Bound Protecting Group)

Maleic anhydride and its derivatives have been implemented by the inventors as an amino protecting group of PEI (and also possibly lysine residues of the carrier). The scheme below depicts a general reaction of a primary amine with a maleic anhydride derivative. As can be seen in the scheme, the amine group, that carries a positive charge at a physiological pH, becomes neutral as it is transformed to an amide group. Simultaneously, the reaction yields a free carboxyl group that is characterized by a negative charge at a physiological pH.

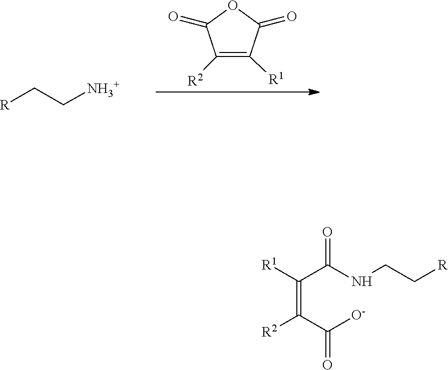

Such anhydride derivatives can react with the primary amines of the PEI modification on the HSA carrier of the invention as well as with the amines of its lysine side chains and N-terminal free amine.

The stability of these protecting groups can be controlled by the substituents on the double bond, where more substituents and larger substituents lead to lower stability. This lower stability can be exploited in this invention to create the desired transient effect of charge masking. As these potential covalent masking agents are less stable at lower pH values, this characteristic can be used to lead to higher accumulation of the masked PEI-modified carrier in tumor microenvironments (TMEs) as such environments are commonly characterized by lower pH than normal, healthy extracellular or plasma environments. While the latter have a pH of about 7.3-7.4, TMEs may have a pH value of about 6.8-7.0 and even lower. Enhanced removal of these masking agents in the TME due to the lower pH will expose the PEI's cations and will lead to cell adhesion and internalization.

The inventors evaluated the effect of charge masking with various anhydride derivatives (Maleic—R1=H, R2=H; Citraconic—R1=Me, R2=H and Dimethyl maleic—R1=Me, R2=Me, cis-aconitic (R1=CH₂COOH, R2=H), 3-(4-Methyl-2,5-dioxo-2,5-dihydrofuran-3-yl)propanoic acid (R1=Me, R2=CH₂CH₂COOH), and 3-Ethylfuran-2,5-dione (R1=CH₂CH₃, R2=H)) using isoelectric focusing (IEF) gel, exhibiting the proteins' pI. The native HSA is characterized by a pI of about 4.8 and the PEI modification leads to extremely high pI, above 8, not measurable using this gel. Both maleic and citraconic anhydrides dramatically lowered the pI of the PEI-modified HSA, even somewhat lower than its native pI. The dimethyl maleic anhydride with its known instability even at pHs around 7 gave a smear probably suggesting removal of this masking prior and during this analysis. Due to this instability, no further work was done with the dimethyl maleic anhydride. The IEF results were corroborated with Zeta potential measurements (Zeta Sizer Ultra, Malvern Instruments). These analyses showed a zeta potential value for native HSA of −14.4. PEI modification (x8) increased the zeta potential to almost +13 while masking with citraconic anhydride lowered the zeta potential of the PEI-modified HSA to −12 when using a molar excess of 85 equivalents.

The masking procedure is as follows:

To a solution of the protein to be masked, at concentrations above 1 mg/mL, the anhydride is added in molar excesses ranging from 50 to 500 equivalents. The reaction is spontaneous and does not require additional catalysts or reagents. The reaction can be maintained from several minutes to hours.

The masking of HSA-PEI×8 with different anhydride derivatives (maleic, citraconic, cis-aconitic (R1=CH₂COOH, R2=H), 3-(4-Methyl-2,5-dioxo-2,5-dihydrofuran-3-yl)propanoic acid (R1=Me, R2=CH₂CH₂COOH), and 3-Ethylfuran-2,5-dione (R1=CH₂CH₃, R2=H)) was calibrated, using different molar excesses of anhydride derivative. Subsequently, the inventors tested the masking efficiency of the above-described anhydride derivatives using IEF gel electrophoresis. The resulting IEF profiles (data not shown) of HSA-PEI×8 masked with different molar excesses of citraconic anhydride, ranging from 75 to 200 equivalents has been compared to native, unmodified HSA. The inventors concluded that no further change in the masked protein's pI can be observed above a molar excess of 100 equivalents. Similar results were obtained with other anhydrides (data not shown). HSA-PEI×8 masked with an 85-molar excess of citraconic anhydride exhibited an IEF profile similar to the native HSA. This masked HSA-PEI×8 was further used in the evaluation of stability of the masking at different pH environments. The citraconic anhydride masked HSA-PEI×8 was incubated at different pHs and samples were withdrawn at 0, 24 and 72 hours for an IEF analysis. The inventors found, that at pH 4 the masking is highly unstable and is practically removed at 24 hours, as compared to the HSA-PEI×8. The inventors noticed that this masking agent exhibits instability even at pH 6. An interesting observation is that this agent is gradually removed even at pH 6.8, a pH that is characteristic of TMEs and the level of removal is far greater than is observed at pH 7.4 where only minor removal is exhibited at 72 hours. This suggests that citraconic anhydride has potential as a covalent masking agent for the PEI-modified carriers of this invention. Maleic anhydride on the contrary, exhibits high stability at all pHs except pH 4 where full removal was observed at 96 hours but only slight removal is observed at the initial hours of exposure to this pH.

Citraconic anhydride is an unsymmetrical anhydride as it has one methyl substitution. In light of this asymmetry, the reaction of citraconic anhydride and an amine can have two structural products which are known as a kinetic product and a thermodynamic product, the earlier is expected to be somewhat less stable due to the resulting steric hindrance. HSA-PEI×8 was masked with citraconic anhydride at thermodynamic conditions (20° C., 2 hours) and kinetic conditions (5° C., 10 minutes). The product of the kinetic conditions is probably not all kinetic product but rather enriched with kinetic product. Both products were further kept at 2-8° C. and further evaluated for their stability by IEF at pHs 7.4 and 6.8, the pH levels relevant in in vivo settings.

The thermodynamic product and kinetic product (at 48 hours of incubation in the corresponding pH) were analyzed side-by-side at each time point in IEF gels (data not shown). The IEF profile of both products seems to be very similar at the beginning of the experiment, however the masked preparation enriched with kinetic product is significantly less stable at all time points.

HSA-PEI×8 was compared to citraconic anhydride masked HSA-PEI×8 in in vivo settings, for evaluation of their pharmacokinetic profiles and biodistribution. The masked HSA-PEI×8 was produced under kinetic conditions, as described above, using a molar excess of 150 equivalents. MALDI-ToF MS analysis was used to quantitate the number of masking agents that were covalently attached to the HSA-PEI×8. Based on MALDI-ToF MS, the inventors concluded that masking led to a noticeable mass shift which was calculated to correspond to the addition of about 44 molecules of citraconic anhydride (see Table 2).

HSA-PEI×3.5 and HSA-PEI×8 were modified with several maleic anhydride derivatives. The level of masking was controlled by the equivalent amount of masking agent in the reaction. The level of masking and the effect on the molecule's charge were evaluated by mass spectrometry and zeta potential, respectively (see zeta potential results in Table 1 and MS results in Table 2). To examine the potential of the masking agent to be cleaved within the tumor microenvironment (reported pH around 6.8), the stability of the masked carrier (HSA-PEI×3.5 or HSA-PEI×8), was tested in vitro, by incubation at different pHs at 37° C. IEF gel was used to evaluate the masking removal. Removal of the masking resulted in an increase of the pI of the protein.

Based on the experimental results, the citraconic anhydride derived masking group was found to be unexpectedly advantageous due to its stability at pH 7.4, and gradual deprotection over time at pH 6.8. Other protecting groups showed inferior stability at pH 7.4 (greater deprotection) or at pH 6.8 (reduced deprotection).

TABLE 1

| | Zeta potential | |
|---|---|---|
| Sample | (mV) | STD |
| HSA | −14.4 | 2.7 |
| HSA-PEI × 8 | 12.8 | 5.2 |
| HSA-PEI × 8 citraconic anhydride (100 eq.) | −9 | 3.7 |
| HSA-PEI × 8 citraconic anhydride (65 eq.) | −9.7 | 3.4 |
| HSA-PEI × 8 citraconic anhydride (85 eq.) | −11.7 | 3.7 |
| HSA-PEI × 8 maleic anhydride (150 eq.) | −12 | 5 |
| HSA-PEI × 8 cis aconitic anhydride (50 eq.) | 1.2 | 1.2 |
| HSA-PEI × 8 cis aconitic anhydride (150 eq.) | −1.8 | 1.1 |
| HSA-PEI × 8 dimethyl maleic anhydride (75 eq.) | 13.1 | 2.8 |

TABLE 2

| | Number of masking |
|---|---|
| Sample | agent |
| HSA-PEI × 3.5 Citraconic anhydride (75 eq.) | 23.8 |
| HSA-PEI × 8 Citraconic anhydride (150 eq.) | 43.8 |

As shown in Table 1, dimethyl maleic anhydride-maskedHSA-PEI has a strongly positive zeta potential, almost identical with the zeta potential of the unmasked HSA-PEI. The inventors postulate that this is due to the instability (rapid deprotection) of the dimethyl maleic anhydride. A similar phenomenon has been observed with aconitic anhydride masked HSA-PEI, which showed a partial deprotection as confirmed by zeta potential values presented in Table 1. Accordingly, the inventors postulate that citraconic anhydride together with maleic anhydride are characterized by a sufficient chemical stability. Moreover, as disclosed above, citraconic anhydride appeared to be preferential due to its stability at neutral pH and above (above 7, or above 7.4), and substantial deprotection at slightly acidic pH of about 6.8 or below.

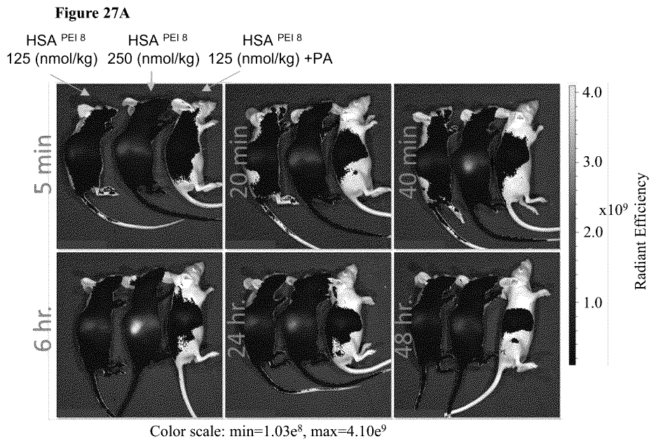

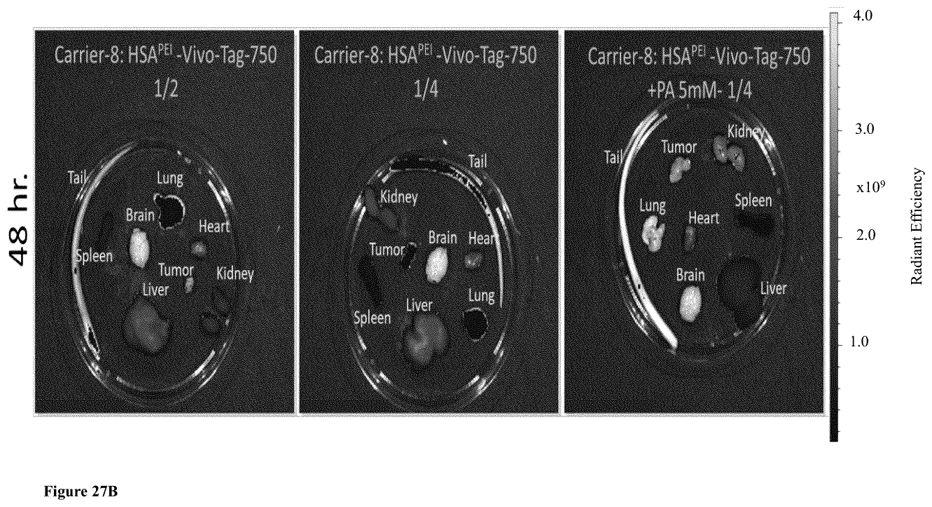

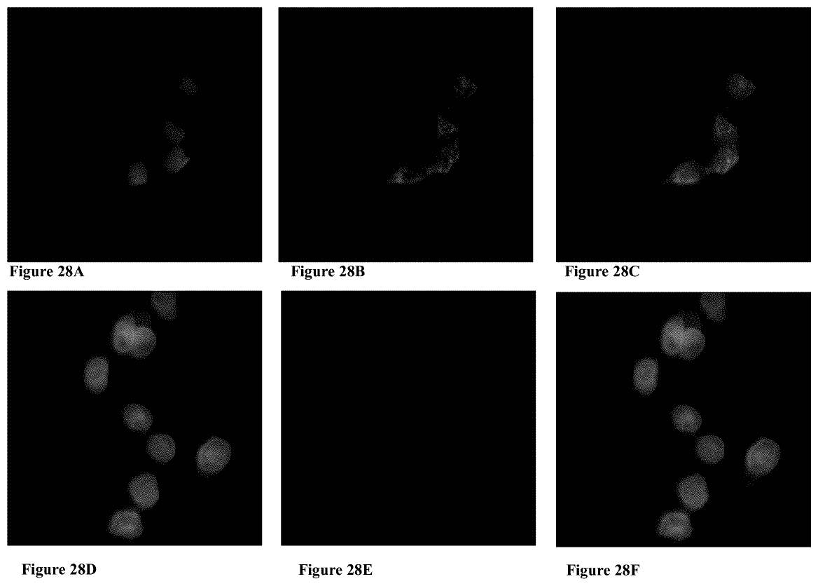

This deprotection that occurs at acidic pH was found to be essential to intracellular delivery. Anti-E7 VHH (described hereinabove) was conjugated to both citraconic anhydride masked and non-masked HSA-PEI×3.5. Both conjugates were evaluated for their ability to internalize into cells. While the non-masked conjugate exhibits clear cell internalization (FIG. 28A-28C) the masked conjugate was not internalized at all (FIG. 28E-28F). This demonstrates that without the positive charges of the PEI modification the conjugate completely loses its ability to adhere to the cell membrane and utilize the endocytosis mechanism for internalization.

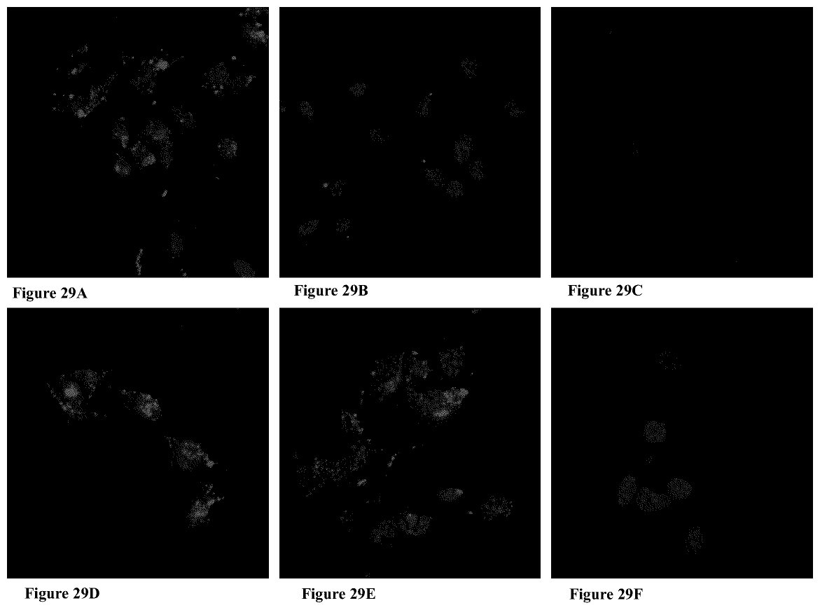

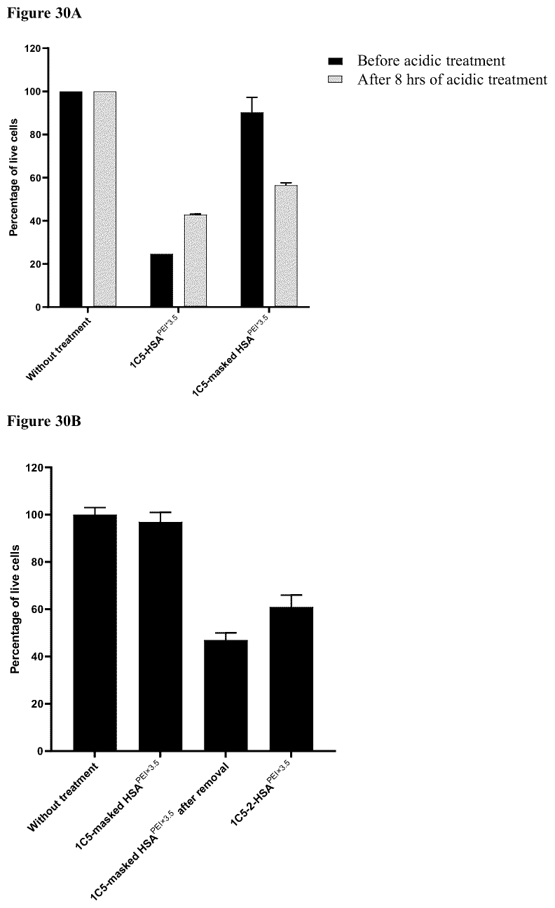

To further test the essentialness of unmasking to internalization, a fluorescently labeled carrier (HSA^{PEI×8}) that was masked with citraconic anhydride (CA, a transient acid sensitive masking agent) or methyl succinic anhydride (MSA, stable masking agent) was used. Fluorescent labeling was performed on HSA^{PEI×8} using 2.5 molar excess of ATTO-542 with a Maleimide moiety (ATTO-TEC, Cat. No. AD 542) directed to the modification on the free Cysteine at position 34 of the HSA. Mass spectra measurements showed that no more than one modification is observed (data not shown). Masking removal was preformed to all three carriers (HSA^{PEI×8}; HSA^{PEI×8} CA; HSA^{PEI×8} MSA) by incubation at pH 4 for 8 h at 37° C. The labeled proteins before and after masking removal were tested for their ability to internalize into B16 melanoma cells during a 24 h incubation. Detection of internalization was by confocal microscopy as before. As can be seen in FIGS. 29A-29F, intracellular fluorescence is observed only from incubations with the unmasked carrier (FIG. 29A, 2D) or the CA masked carrier following masking removal (FIG. 29E). Indeed, the carrier masked with the non-transient, stable masking was not able to enter the cells even after the acidic treatment (FIG. 29C, 29F).

Next, it was checked if a payload-masked carrier conjugate after in vitro mask removal (incubation at pH 4 at 37° C. for 8 hr.), can not only internalize into cells but also exhibit functionally there. This in vitro treatment was used to mimic the effect of the acidic conditions found in the tumor environment, as well as other acidic environments occurring in various health conditions. To test functionality an anti-BRAF VHH, 1C5, conjugated via a linker to a citraconic anhydride masked carrier carrying an average of 3.5 PEI modifications (1C5-Hel-masked HSA^{PEI×3.5}) was used. Different cancer cell lines were treated with this agent, before and after in vitro masking removal (pH 4, 37° C., 1 or 8 hours). Its non-masked counterpart was used as a control. As can be seen in FIG. 30A, the non-masked conjugate had a cytotoxic effect on the treated MEL-526 melanoma cells as the intracellular delivery of the anti-BRAF agent would be expected to. Masking removal in this case had no effect as there was no mask to remove. The masked conjugate however, had no effect on the treated cells, as masking efficiently prevented internalization (FIG. 30A). The in vitro acidic treatment was able to remove the masking, exposing the PEI modifications, and re-enabled the internalization of the conjugate to the cells. This can be seen by the cell killing, which is very close to the killing achieved with the non-masked conjugate (FIG. 30A). As can be seen in FIG. 30B, a similar result was observed in SK-MEL-28 melanoma cells. Here also, the anti-BRAF 1C5 non-reversibly conjugated to a masked carrier (3.5 PEIs) had no effect on these cells while the same agent following in vitro acidic treatment had a dramatic cytotoxic effect on these cells, an effect similar in its magnitude to the one produced by non-masked conjugate.

An in vivo experiment in mice was performed to confirm the effect of the masking. HSA-PEI×3.5, HSA-PEI×8 and HSA-PEI×8 masked with citraconic anhydride were injected (58 nmol/Kg) IV to C57 mice subcutaneously engrafted with B-16 mice melanoma cancer cells. Engraftment was carried out 2 weeks prior to administration of the different HSA-PEI derivatives. Each derivative was injected to 15 mice and at each time point 3 animals were bled. At some time-points, animals were also sacrificed, and different organs were obtained for biodistribution analysis. The level of HSA-PEI was determined in both plasma and organ lysates using an in-house ELISA. As the citraconic anhydride masking also interferes with the detection by ELISA, samples of animals injected with the masked HSA-PEI were treated at pH 4 for about 1 hour prior to their ELISA analysis for full removal of the masking agents.

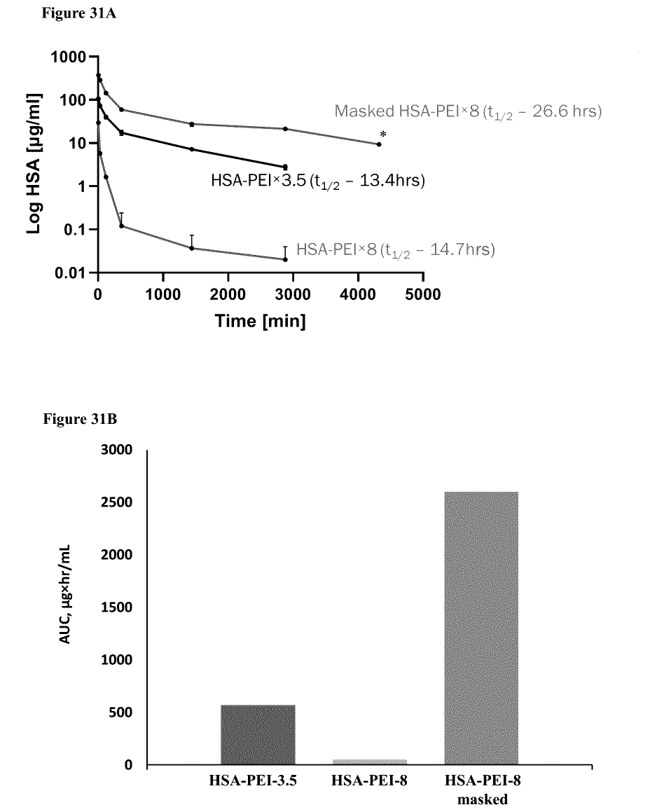

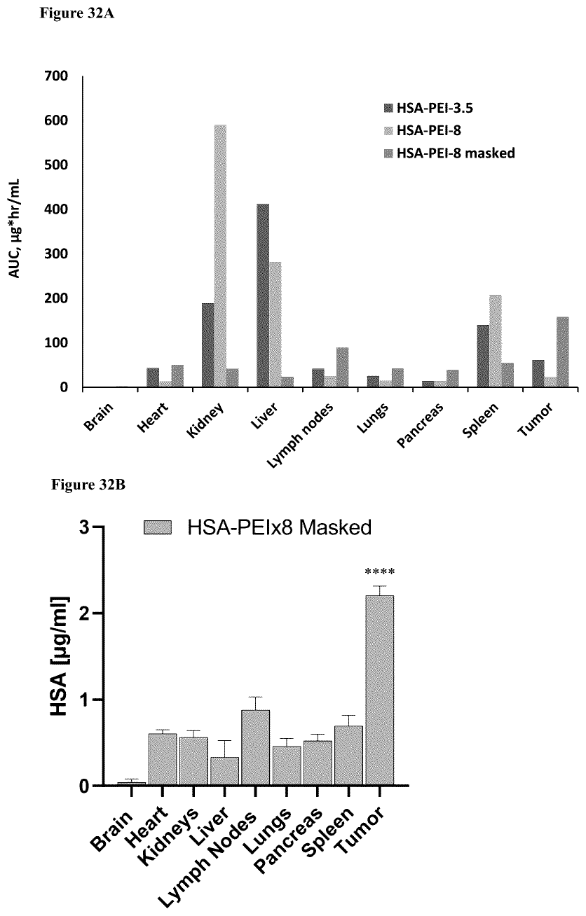

The obtained pharmacokinetic profiles of the HSA-PEI derivatives can be seen in FIG. 31A). HSA-PEI×8 is characterized by very quick clearance from the plasma. Some residual quantity is cleared more slowly and leads to an apparent half-life of about 15 hours. In contrast, the same HSA-PEI×8 masked with citraconic anhydride is cleared much slower and gives a half-life of almost 27 hours. The masking effect is even more dramatic when one examines the plasma exposure which is calculated as the area under the curve (AUC). The plasma exposure of the masked HSA-PEI×8 is 50 times higher than the exposure level of the unmasked HSA-PEI×8. The HSA-PEI×3.5 gave a half-life of 13 hours and its PK profile seems to be more favorable than the HSA-PEI×8. But still, its plasma exposure is 5-fold lower than the masked HSA-PEI×8 (FIG. 31B).

Examination of the levels of the different HSA-PEI derivatives in the animals' organs provides further insight into the low plasma exposure of the PEI modified HSA. As can be seen in FIG. 32A, in the organs exposure levels (AUC), both the HSA-PEI×3.5 and the HSA-PEI×8 seem to undergo some sort of entrapment in the clearance organs, namely liver, kidney and spleen. Most of the injected amount of the PEI-modified HSA can be found in those organs. In contrast, the citraconic anhydride masking eliminates this entrapment and enables the masked HSA-PEI×8 to circulate longer in the animals' blood stream and to distribute more homogenously in the different organs and tissues. This finding is highly surprising and important. The ability to steer the composition away from undesired organs is essential for therapeutic efficacy. It also allows delivery of much lower doses. And both of these aspects will decrease unwanted side effects and off-target effects. Furthermore, while the distribution of the masked agent seems to be rather homogenous in the organs, one tissue seems to be more exposed to the masked agent and this is the tumor (FIG. 32A). The tumor was exposed to 2-7-fold more masked HSA-PEI than other organs (FIG. 32B). This is due to the sensitivity of the masking agent to the acidic environment of the tumor which leads to its reversal, exposing the positively charged PEI modifications which in turn leads to cell adherence and the start of cytosolic delivery. While the inventors have shown above that even at the neutral pH of the plasma the masking undergoes slow reversal, this effect is more rapid in the more acidic tumor environment which leads to enrichment of the carrier of the invention in this specific environment.

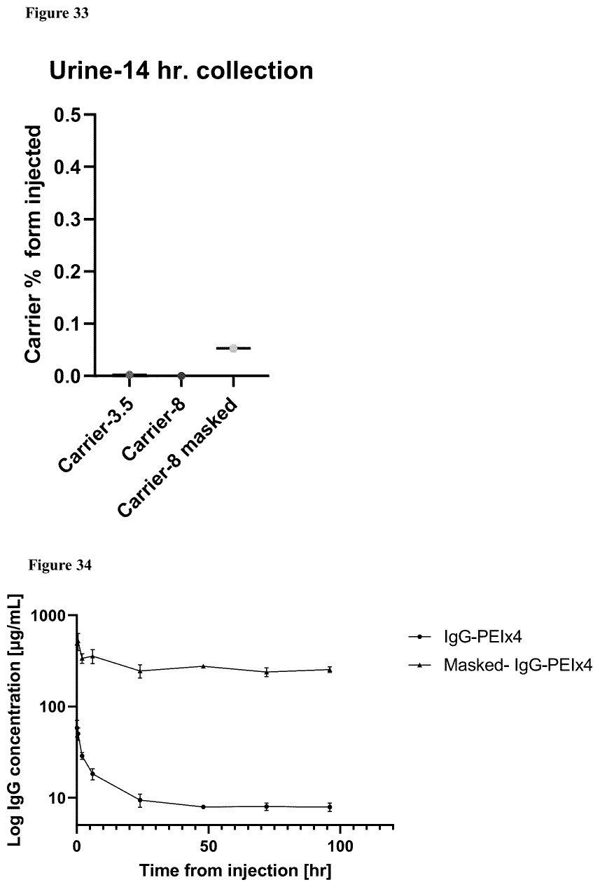

In order to ensure that the low amounts of the masked HSA-PEI×8 found in the kidneys is not the result of clearance through urine, animals in this in vivo study have been treated in metabolic cages and their urine was collected during the first 14 hours of the experiment. As shown in FIG. 33, only extremely minute amounts of the injected carriers could be found in the urine of the administered animals, less than 0.1% of the initial dose. All of this data taken together emphasizes that while protection of the PEI is necessary to ensure sufficient serum half-life and proper biodistribution, deprotection of the PEI is necessary at the target cells (e.g., tumor) in order to facilitate intracellular delivery.

### Example 11: In-Vivo Results of Protein Conjugates with Additional Protein Carriers

To evaluate in vivo compatibility of alternative carriers (which are not HSA), an IgG protein (Humira®, Abbvie) was modified with PEI (600 Da) in the presence of EDC to give 4 PEI modifications, named IgG-PEI×4. The modified IgG protein was further reacted with citraconic anhydride to yield masking of the PEI's positive charges, named: masked-IgG-PEI×4.

The PK, Zeta potential and mass of the two IgG carriers, IgG-PEI×4 and masked IgG-PEI×4, were evaluated. As can be seen in Table 3, PEI modification dramatically increased the Zeta potential value of the IgG while masking changed this value to a negative value, far below the Zeta potential value of the non-modified original IgG.

TABLE 3

| | | | Masked-IgG- |
|---|---|---|---|
| | IgG (Humira) | IgG-PEI × 4 | PEI × 4 |
| Zeta potential (mV) | 12.4 ± 3 | 36 ± 3 | −13.5 ± 4 |
| AUC-0t [μg/mL*h] | Not tested | 1016 | 26,065 |
| Clearance [mL/kg/h] | Not tested | 8.8 | 0.18 |

Balb-C mice were IV injected with test samples IgG-PEI×4 or Masked-IgG-PEI×4 at a dose of 120 nmol/Kg. At the tested time points, mice were bled and the sample concentration in the blood was evaluated by Sandwich ELISA (coating: Goat anti Human FAB2 (Jackson, Cat. No. 109-005-097; detection: Donkey anti human FC HRP (Jackson, Cat. No. 709-035-098). As can be seen in the FIG. 34 (and Table 3), the IgG carrier, modified with PEI, exhibited similar PK parameters (AUC and CL) as the HSA-PEI×3.5, showing the characteristic fast elimination, low AUC and high clearance. As with the HSA carrier, the masked IgG carrier restores its long half-life, very high AUC and low clearance, again exhibiting that the masking with maleic anhydride derivatives, and specifically citraconic anhydride, confers PEI-modified carriers of the invention, such as IgGs, with clinically favorable pharmacokinetic profiles.

### Example 12: In-Vivo Results of PEI-Modified Antibody without a Protein Carrier

To further examine the ability of the transient masking to eliminate high clearance and short plasma half-lives, single domain antibodies that were directly modified with PEI were further masked with citraconic anhydride. Specifically, the active anti-E7 VHH described hereinabove, was produced without a carrier, and modified with PEI (1800 Da) at the payload's C terminus (a GGGGSC linker was added at the C-terminus), in a non-reversible manner, referred to here as VHH αE7-PEI1800. The PEI modified VHH was further masked with citraconic anhydride to yield a masked PEI-modified protein, named: masked-VHH αE7-PEI1800.

The Zeta potential of the two compounds: VHH α E7-PEI1800 and masked-VHH α E7-PEI1800, was evaluated and compared to the native non-modified VHH. Further, the PK of the PEI modified VHH and its masked counterpart was evaluated in mice. Balb-C mice were IV injected with the tested samples (VHH α E7-PEI1800 or masked-VHH α E7-PEI1800) at a dose of 120 nmol/Kg. At the tested time points, mice were bled and the sample concentration in the blood was evaluated by Sandwich ELISA (coating: Streptavidin (Prospec, Cat. No Pro-791-b), followed by Rabbit anti VHH+biotin (A2S, Cat. No. A01995-200), detection: Rabbit anti VHH cocktail-HRP (A2S, Cat. No. A02016-200).

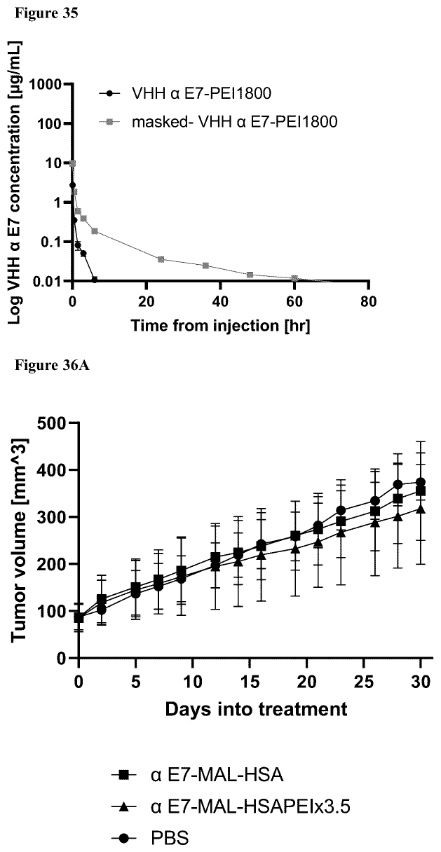

As can be seen below in Table 4 and FIG. 35, the PEI modified VHH had very high clearance from the plasma of mice and extremely low AUC. Masking of the positive charges, shown to reverse the detrimental PK effects of the PEI modification in the carriers of the invention, improved the PK parameters of the PEI modified VHH. While the effect of masking on larger carriers, HSA and IgG for example, was very pronounced, the effect on a small PEI-modified protein was less so. While the masking itself abrogated the effects of the strong-positive charges, i.e., capturing in the liver, spleen and kidneys, its small size still led to high clearance for simpler size-related reasons.

To this end, the inventors postulated that in addition to the use of masking agents the protein conjugate of the invention has to include a carrier to facilitate delivery of a payload to the target site within a subject.

TABLE 4

| | | VHH αE7- | masked-VHH αE7- |
|---|---|---|---|
| | Native αE7 | PEI1800 | PEI1800 |
| Zeta potential (mV) | −5.9 ± 2 | 4.9 ± 3 | −5.3 ± 2 |
| AUC-0-t [μg/mL*h] | N.T. | 1.5 | 9 |
| Clearance [mL/kg/h] | N.T. | 933 | 179 |

### Example 13: Anti-HPV-E7 VHH In Vivo Results

The previously described anti-HPV-E7 VHH was conjugated to HSA-PEI×3.5 via a non-cleavable linker comprising a PEG₁₁ chain and with two terminal maleimide groups, both reactive towards thiol groups. The VHH with a C-terminal cysteine (GGGGSC linker at the C-terminus) was treated with a reducing agent (TCEP) to free the terminal cysteine and was then reacted with the Bis-Mal agent. Following chromatographic purification, the VHH with now a terminal maleimide group was reacted with HSA that was pre-modified with PEI (an average of 3.5 per HSA) and citraconic acid for masking, so as to obtain an exemplary protein conjugate of the invention (αE7-VHH-S-Mal-PEG₁₁-Mal-S-HSA×3.5) schematically presented below:

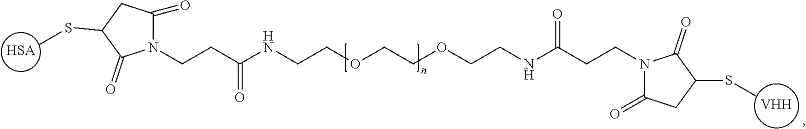

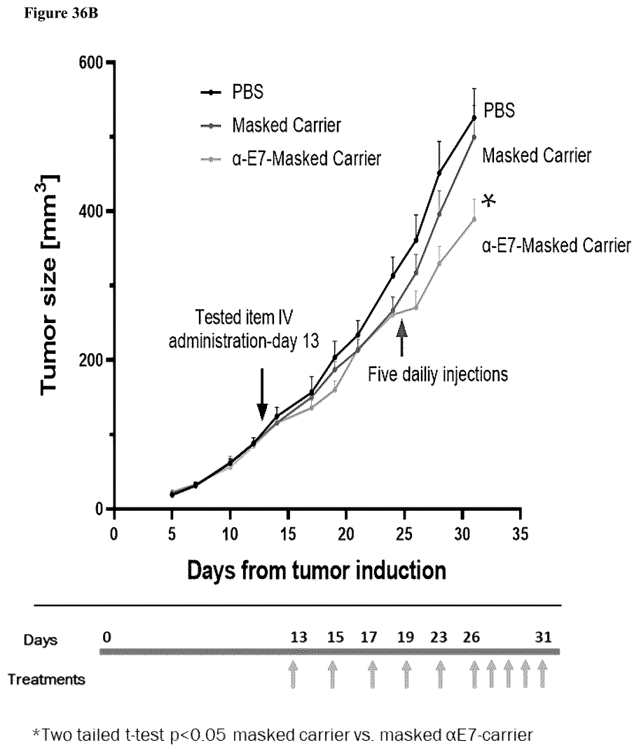

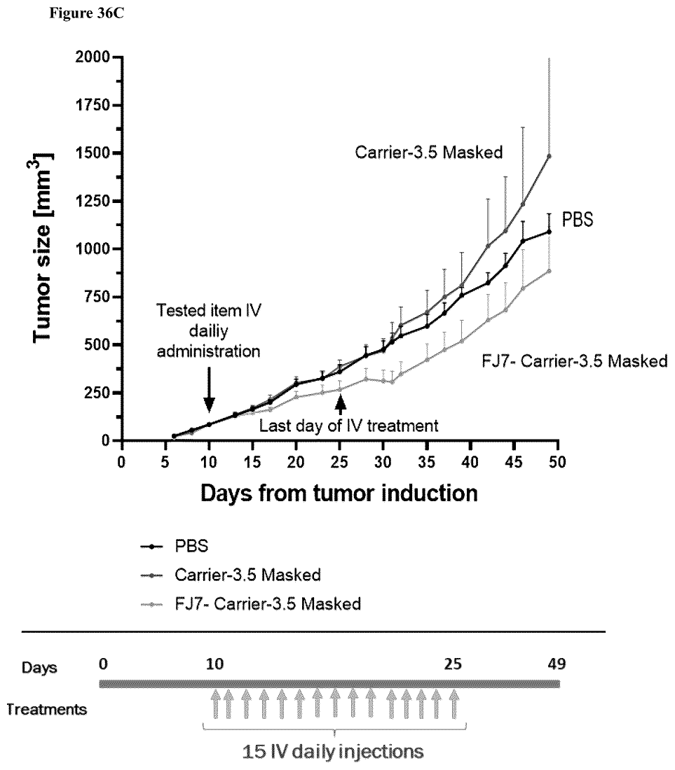

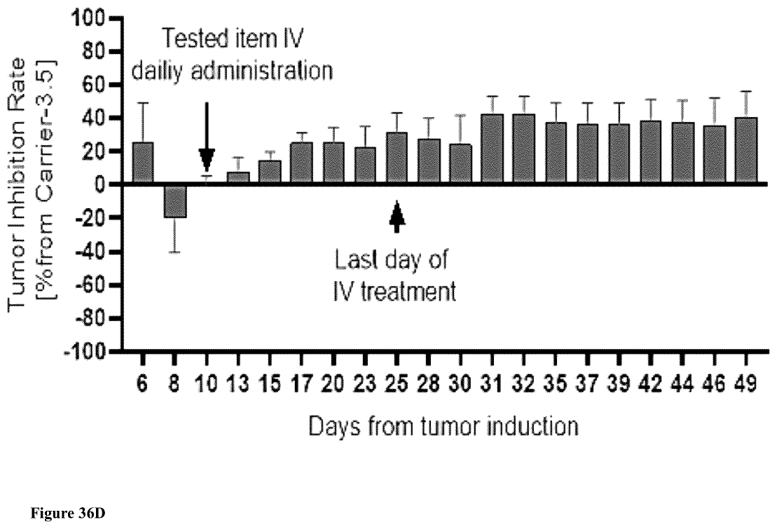

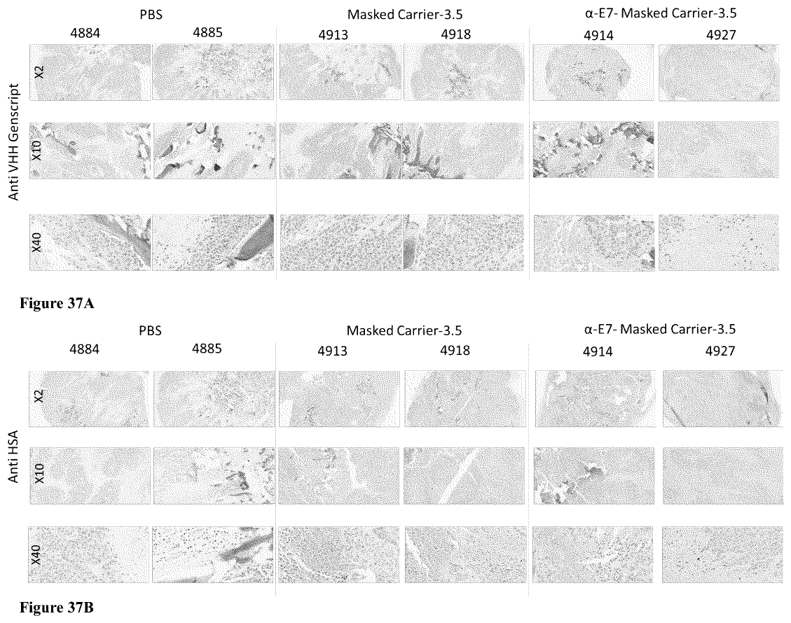

The resulting masked αE7-VHH-S-Mal-PEG₁₁-Mal-S-HSA×3.5 was used to treat athymic nude mice engrafted subcutaneously with HeLa-GFP cells. The same molecule without the PEI modification was used as a negative control. The conjugate of the invention with no PEI modification had no effect on tumor growth as expected (FIG. 36A). The PEI modified conjugate had a minimal effect on tumor growth, suggestion that without masking the availability of the agent to tumor cells is minimal. This is probably due to the stickiness of the positively charged conjugate, as exemplified above in the poor pharmacokinetic and biodistribution profiles exemplified for non-masked carrier (FIG. 31A-31B and 32A).

Next the masked αE7-VHH-S-Mal-PEG₁₁-Mal-S-HSA×3.5 was injected intravenously (250 nmol/Kg) to the mice following tumor engraftment starting when the tumors achieved a mean volume of about 100 mm³. Treatment included periodic administration of the agent and controls. Citraconic anhydride masked HSA×3.5, and vehicle (PBS) were administered as controls. The mice treated with the masked αE7-VHH-S-Mal-PEG₁₁-Mal-S-HSA×3.5 exhibited significant tumor growth inhibition as compared to the controls (FIG. 36B, two tailed t-test p<0.05 masked carrier vs. masked αE7-carrier). Additionally, the treatment was well tolerated by all animals, as evidenced by the lack of any adverse events and normal weights of the animals (data not shown).

The somewhat limited tumor growth inhibition can be attributed to the fact that the anti-E7 VHH has limited affinity to the E7 protein, with an estimated K_{D} above 1 μM.

The experiment was repeated following the same protocol with the following changes: 1) the administered dose of agents was raised to 350 nmol/Kg and 2) the injections were carried out every day for 15 consecutive days. Animals continued to be monitored for tumor volume for an additional 24 days. As can be seen in FIG. 36C, a similar tumor growth inhibition as in the first study was observed when examining the tumor volume of treated animals in which initial tumor volume at the beginning of treatment was below 97 mm³. As expected from the higher dosage and higher frequency of administration, the tumor growth inhibition was initially observed at an earlier time point and the magnitude of the effect was also larger.

Surprisingly, the inhibition effect was persistent even after the administration of agents had been stopped (FIG. 36D). Indeed, the full effect was maintained for another 5 days and was even present 24 days after the cessation of treatment. The anti-E7 agent used here was permanently linked to the carrier. We have observed in the past that the carrier generates clusters or aggregates upon escaping from the endosomes (see also FIG. 28B). This may explain the persistent inhibition effect, as the aggregates may act as a sort of a depot that slowly releases additional agent into the cytoplasm over time.

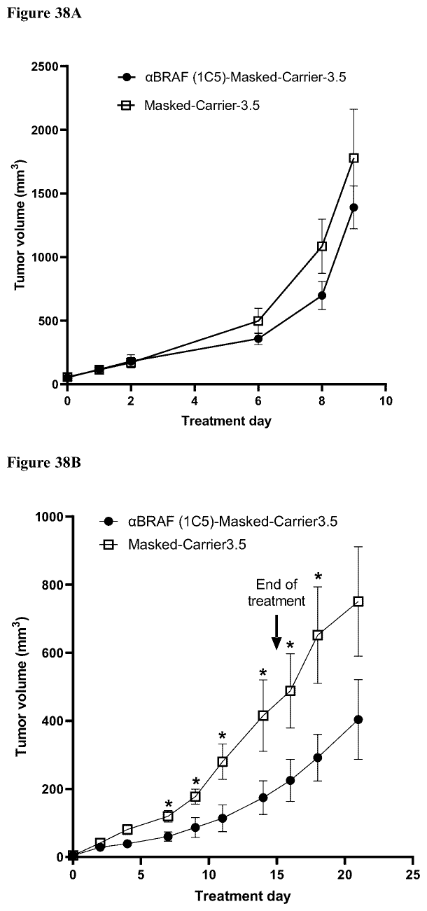

Tumors from the treated animals of the initial study were obtained and analyzed for the presence of the VHH agent and HSA carrier using immunohistochemistry. Cross-sections were stained by H&E and by antibodies for the VHH or for HSA^{PEI} (antibody raised against PEI modified HSA and not HSA itself). Tumors from animals treated with PBS (FIG. 37A-37B, left) show no staining for either VHH or carrier. Tumors from animals treated with the masked carrier show clear and strong staining for the carrier but not for the VHH payload (FIG. 37A-37B, middle). Tumors from animals treated with the anti-E7 VHH conjugated to the masked carrier clearly show staining for both the carrier and the VHH payload (FIG. 37A-37B, right). Most interestingly, the staining is more pronounced in areas of tumor necrosis. Furthermore, presence of both the carrier and the full conjugate is observed throughout the tumor and is not confined to specific areas, for example, close to major blood vessels. This latter observation suggests an efficient distribution of the payload-carrier conjugate throughout the tumor tissue.

To verify that the staining observed in the tumor tissue is indeed specific staining and not background staining of necrotic areas, isotope control staining was performed for all tissue tested and compared between all treatments. Negative staining in all samples was observed when using the isotype-control (anti-rabbit IC Rabbit (DA1E) mAb IgG XP Isotype Control #3900, Normal Goat IgG, sigma N102-100UG) (data not shown). This confirms specific necrotic area staining.

It should be emphasized that detection of the carrier in these sections was achieved by using a polyclonal antibody that was raised against PEI-modified HSA which does not cross-react with non-modified HSA. Additionally, the masking of PEI-modified HSA with citraconic anhydride practically renders the masked carrier to be undetectable by this polyclonal antibody. Thus, the fact that the carrier, PEI-modified HSA, is clearly detected in these tumor sections indicates that indeed the masking was removed in the tumor microenvironment enabling the agent of the invention to enter the cells using its exposed PEI modifications.

### Example 14: Anti-BRAF VHH In Vivo Results

The in vivo efficacy of intracellular delivery using a masked agent was also tested with a second VHH. An anti-BRAF VHH (1C5) was non-reversibly conjugated to masked HSA (3.5×PEI). The toxicity of 1C5-Mal-PEG₁₁-Mal-masked-HSA-PEI×3.5 on mice was evaluated and compared to the effect of just the carrier (masked HSA-PEI×3.5). Mice of two strains (C57BL and NOD-Scid) underwent a dosage escalation routine. The tested agents were injected intravenously, with 200 μL of agent administered per injection. Dosage escalation started at 50,100,250, and 350 nmol/Kg, every other day, followed by three 350 nmol/Kg IV injections every other day, and finally five daily IV injections of 350 nmol/Kg. Mice were monitored for clinical signs of morbidity or mortality such as changes in skin, fur, eyes, mucous membrane, gait, occurrence of secretions/excretions, decrease of body weight and overall wellness. No clinical signs were observed in any of the tested mice. In addition, at the end of the dose escalation and repeated dosing, all mice underwent terminal anesthesia with a ketamine-xylazine cocktail (IP). Animals were perfusion-fixed via the heart using PBS, followed by 4% PFA. Organs were collected (liver, heart, kidneys, lungs, brain, spleen) and stained with Hematoxylin & Eosin (H&E). No pathological changes were found between the two groups.

The payload used in this evaluation is a non-selective anti-BRAF agent, which was shown to inhibit both wildtype and mutated BRAF, of both human and murine origin. The inventors have already shown that the payload-masked carrier conjugates of the invention are biodistributed to practically all organs and tissues following administration. Thus, it is somewhat surprising that such a non-selective inhibitor of a key cellular enzyme has no evident toxic effect on treated animals. However, it should be noted that the masking counteracts the effect of the positive charges and hence prevents intracellular delivery unless the masking agent is removed. As the masking is pH sensitive, the biodistribution results showed that more of the conjugates reached tumor tissues which is characterized by a lower pH environment than normal healthy organs and tissues. It is thus the inventors' hypothesis that while the conjugate is reaching all organs and tissues, it there encounters pH conditions which are neutral or slightly above neutral (physiological pH), and hence the masking is relatively stable, and the internalization of the conjugate and its allegedly toxic payload is inhibited. As these animals had no inoculated tumors, the conjugates did not encounter an acidic tumor microenvironment and did not exhibit cell internalization. Even if some masking removal is taking place, to some small degree, in healthy organs and tissues, the amounts that will enter such cells appear to be quite small and ineffective.

To further this point, the ability of 1C5-Mal-PEG₁₁-Mal-masked-HSA^{PEI×3.5} to inhibit tumor growth compared to the carrier (masked HSA^{PEI×3.5}) was evaluated in mice of both strains. C57BL mice were injected subcutaneously with B16 tumor cells (murine melanoma) and NOD-Scid mice were injected subcutaneously with MEL-526 (human BRAF-overexpressing melanoma cells). Testing in the C57BL mice was performed when the tumors were well established (115 mm³), while testing in the NOD-Scid mice was performed while the tumors were in early growth stage (approx. 6 mm³). These models thus represent the ability to treat as well as prevent cancer. In both cases the tested agent, 1C5-Mal-PEG₁₁-Mal-masked-HSA^{PEI×3.5}, exhibited inhibition of tumor growth in comparison to just the payload-free masked HSA^{PEI×3.5} (FIG. 38A-38B). The 1C5-Mal-PEG₁₁-Mal-masked-HSA^{PEI×3.5} (αBRAF-M-Carrier) had a modest inhibitory effect on the growth of the B16 tumors (FIG. 38A). It should be noted that the B16 tumors were highly aggressive, and their rate of growth was rapid. Thus, even this modest growth inhibition still represents a substantial functional effect of the masked conjugate on tumor growth. In the case of the MEL-526 tumors inoculated to NOD-Scid mice, the masked conjugate had a more significant inhibitory effect on the tumors, showing 47% reduction in tumor volume at the end of measurement (FIG. 38B).

This data further demonstrates the selective removal of the masking in acidic environments, such as in tumors. The nonspecific anti-BRAF VHH had no measurable effect on healthy mice but produced an antitumor effect due to exposure to the acidic TME. This enables action-site specificity and the use of even non-selective payloads, conferring cytotoxicity or biological effects only on cells that are found in these low-pH environments. Additionally, this suggests that while the conjugates of the invention reach all organs and tissues, they practically do not enter the cells of these organs and tissues. As the payload is directed against an intracellular target, no effect is exhibited by these conjugates in healthy tissue, greatly reducing the risk of off-target effects.

### Example 15: PK and Biodistribution of Different Constructs with αE7 VHH Payload

The pharmacokinetic profiles and biodistribution of different masked constructs were evaluated in mice inoculated with B16 tumors. All constructs carried the anti-E7 VHH as payload (with a G4SC linker, either 1 repeat as in the MAL constructs or 3 repeats as in the PEP constructs). This VHH was conjugated to carriers modified by either 3.5 or 8 PEIs, and masked with the transient masking agent, citraconic anhydride (CA). The VHH was conjugated to these carriers either via a reversible, disulfide bond, or a non-reversible bond, utilizing maleimide chemistry. The anti-E7 VHH was also conjugated non-reversibly to carrier modified with 8 PEIs that was pre-masked with a non-reversible masking agent, methyl succinic anhydride (MSA), which resembles the citraconic anhydride in its structure but lacks the double bond.

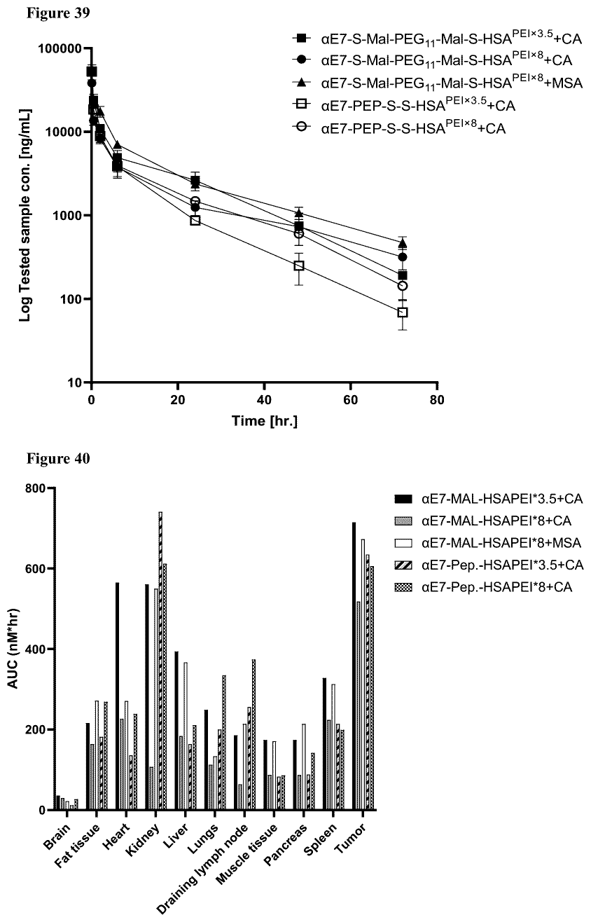

C57BL mice bearing B16 tumors were used to evaluate the biodistribution and pharmacokinetic profile of the agents described above. Once tumors developed to an average volume of 105 mm³, mice received a single IV injection of the various agents at 250 nmol/Kg. Plasma and organ samples were collected at 5 and 30 minutes and 2, 6, 24, 48, and 72 hours post injection. Analysis of the constructs in both plasma and organs was carried out on the payload alone, using an in-house ELISA for VHH. FIG. 39 depicts the pharmacokinetic profiles of the different constructs in the inoculated animals. Table 5 summarizes the pharmacokinetic parameters of each construct.

TABLE 5
Terminal half-life (t_{1/2}) predicted area under the curve
(AUC_{0→∞}) and clearance values of the different constructs
according to a non-compartmental plasma pharmacokinetic analysis.

| | | AUC_{0→∞} | Clearance |
|---|---|---|---|
| Tested Construct | t_{1/2} (hr) | (μM*hr) | (mL/hr) |
| αE7-S-Mal-PEG₁₁-Mal- | 12.7 | 14.3 | 0.348 |
| S-HSA^{PEI×3.5} + CA | | | |
| αE7-S-Mal-PEG₁₁-Mal- | 18.5 | 10.2 | 0.488 |
| S-HSA^{PEI×8} + CA | | | |
| αE7-S-Mal-PEG₁₁-Mal- | 19.4 | 18.2 | 0.274 |
| S-HSA^{PEI×8} + MSA | | | |
| αE7-pep-S-S-HSA^{PEI×3.5} + CA | 12.6 | 9.1 | 0.544 |
| αE7-pep-S-S-HSA^{PEI×8} + CA | 13.6 | 11.4 | 0.435 |

The pharmacokinetic profiles and parameters are quite similar between the different constructs. This is probably the results of the similar pI of the final constructs and their practically identical size. Importantly, the effect of masking is essentially the same regardless of the level of PEI modification. Similarly, the different linkers do not seem to have any effect. It should be noted that the construct masked with MSA indeed shows somewhat higher residency time in plasma and lower clearance, probably due to the fact that its masking is not removed and hence its uptake into cells is minimal to non-existent. It is thus left at higher levels in the plasma.

Different organs, including tumors, were harvested and the level of VHH was analyzed by an appropriate in-house VHH ELISA. FIG. 40 summarizes the organ exposure of the different conjugates. These biodistribution data do not show dramatic differences between the conjugates. It should be noted that the kidney exposure data for the reversible linker conjugates is over estimated as the materials contained a contamination of free VHH. While this free VHH is cleared quickly from the circulation through the kidneys, the early measurement in this organ do measure this free VHH and affect the total AUC analysis. In all cases it can be seen that there is enrichment of payload in the tumor when compared to other organs. This is in line with the enrichment observed for the carrier alone.

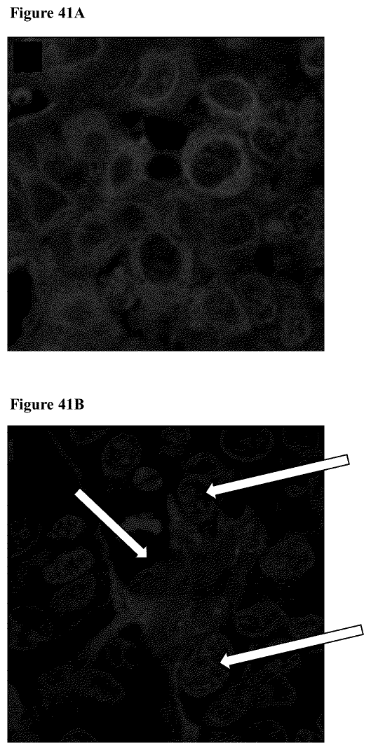

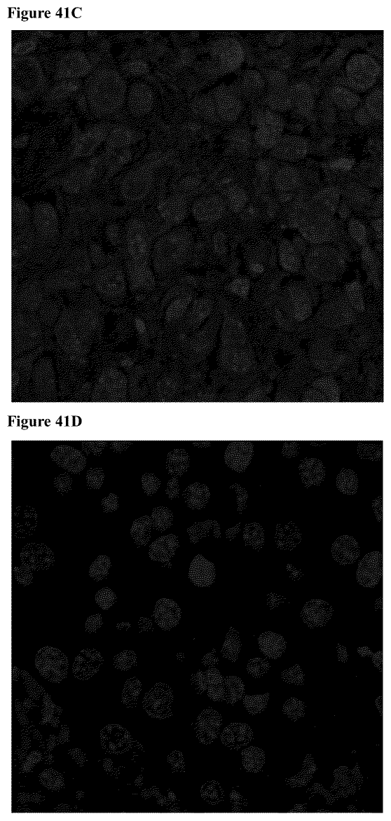

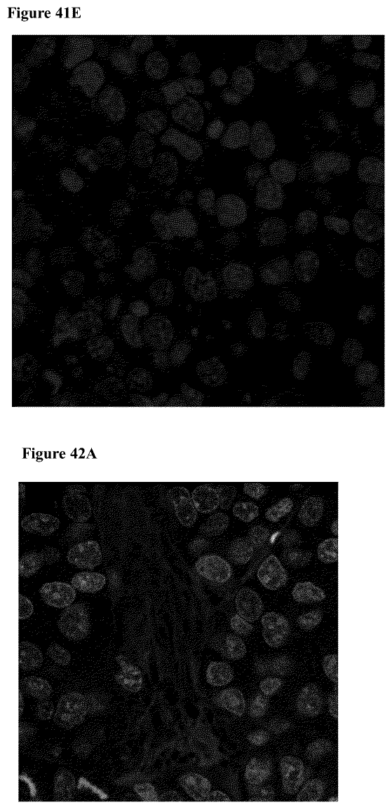

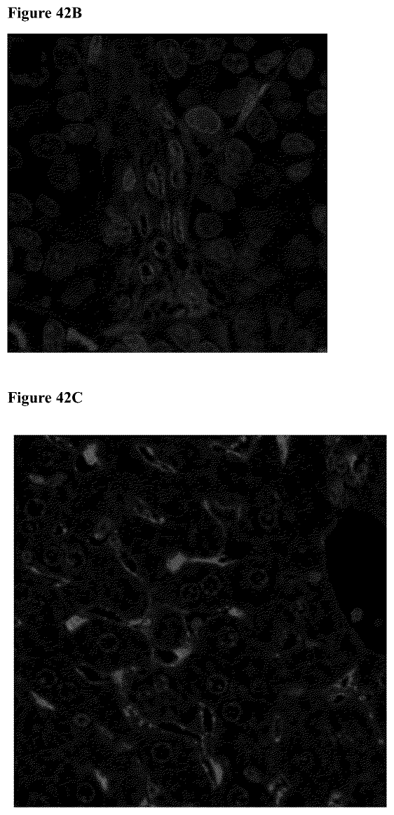

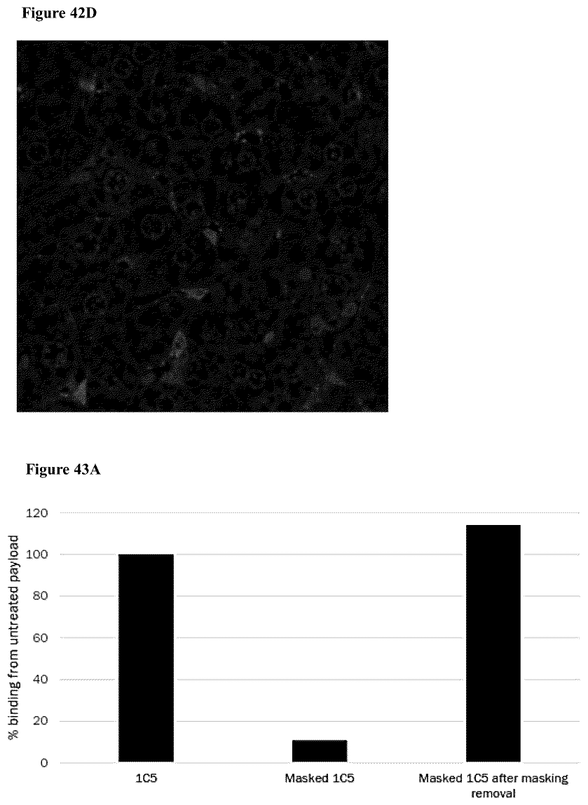

While the pharmacokinetic and biodistribution data does not show dramatic differences between the different masked conjugates, differing in PEI levels and reversibility of linker, the ELISA shows only the total amount of agent present in a tissue/tumor but cannot differentiate between external and cell internalized payload. To this end the above described fluorescently labeled carrier molecules were tested in vivo. HSA^{PEI×8} CA and HSA^{PEI×8} MSA were injected to athymic nude Foxn1nu mice with a Hela-GPF tumor. The different carriers were injected at 180 nmol/Kg dose in 200 μL, and after 6 hours tumors were harvested, and the distribution of the different carriers was evaluated by confocal microscopy. As can be seen in FIG. 41A, the fluorescently labeled CA masked carrier (HSA^{PEI×8} CA) is clearly detected inside the cells of the tumor (tumor cells were identified by GFP fluorescence). Indeed, the carrier is seen enveloping the nuclei of the cells, demonstrating it is dispersed in the cytoplasm of these cells. In contrast, the fluorescently labeled MSA masked carrier (HSA^{PEI×8} MSA) is only detected outside the cells and between the cells, but not in their cytoplasm (FIG. 41B, arrows indicate cytoplasm around tumor cells that is not stained red). The effectiveness of the transient masking is clearly demonstrated, as the carrier cannot effectively enter the tumor cells with the permanent masking. Similar results were observed in B16 tumors in mice treated with CA-masked conjugate (FIG. 41C), MSA-masked conjugate (FIG. 41D) and unmasked conjugate (FIG. 42E). Although tumor cells did not fluoresce in this tumor, it is clear that the conjugate only significantly entered cells with the transient masking and not with the permanent masking. Further, masking clearly enriched the amount of conjugate that arrived at the tumor.

Furthermore, as can be seen in FIG. 42A-42B, the MSA masked carrier is practically not detected in the tumor cells. Tumor cells are characterized by double staining of their nuclei (green and blue). However, the carrier can be easily detected only in areas containing non-tumor mouse cells (characterized by single blue nuclei staining). Based on their shape and morphology, these cells are endothelial cells of blood vessels. This suggests that due to the non-transient character of this masking the negatively charged masked carrier cannot efficiently cross the endothelial barrier and distribute into the tumor tissue and into the tumor cells. When non-tumor tissue, that is tissue without an acidic microenvironment, was examined (e.g., the liver), localization of the conjugate was similar between the two masking agents in that it is not able to enter cells and is localized primarily between cells (FIG. 42C-42D). Taken together this data emphasizes that while the pharmacokinetic and biodistribution profiles of transiently masked carriers and conjugates is very similar to the corresponding profiles of the non-transient masked conjugate there is a dramatic difference in their distribution in tumor tissues and in their capacity to internalize into the tumor target cells, while sparing exposure to healthy cells. These differences cannot be ascertained by using detection tools, such as ELISA, that cannot discriminate between moieties that are inside or outside of cells.

### Example 16: Full Masking

Until now only masking on the carrier itself has been tested. For the purpose of increasing even further the selectivity of activity, the masking of the whole conjugate, including the payload, was tested. Such masking may lower or even completely inhibit the activity of the payload till this masking is removed near the site of action, or only after internalization to the cells themselves following further masking removal in the endosomes, thus avoiding off-target effects. Masking of the whole conjugate can also have various process related advantages. For this purpose, 1C5-S-Mal-PEG₁₁-Mal-S-HSA-PEI×3.5 and 1C5-S-Mal-PEG₁₁-Mal-S-HSA-PEI×8 were masked at different masking levels, controlled by the excess of citraconic anhydride (CA) used in the masking step. The resulting masking levels were evaluated by IEF and by Zeta potential measurements. Additionally, the number of masking moieties that were conjugated to the agents were also analyzed and quantified by MALDI-ToF mass spectrometry. As can be seen in the Table 6 below, which was corroborated in corresponding IEF gels (data not shown), the masking of the full conjugate was also able to counteract the PEI modification and to yield negative charges, thus reducing the Zeta potential of these conjugates.

TABLE 6

| | | Number of |
|---|---|---|
| | Zeta potential | masking |
| Construct | [mV] | agent units* |
| 1C5 | −3 ± 1 | 0 |
| 1C5-S-Mal-PEG₁₁-Mal-S-HSA-PEI × 3.5 | 5.6 ± 2.9 | N.T. |
| | | (not tested) |
| 1C5-S-Mal-PEG₁₁-Mal-S-HSA-PEI × 3.5/ | −4 ± 0.6 | N.T. |
| CA at 125 molar excess | | |
| 1C5-S-Mal-PEG₁₁-Mal-S-HSA-PEI × 8 | 7 ± 2 | 0 |
| 1C5-S-Mal-PEG₁₁-Mal-S-HSA-PEI × 8/ | −9 ± 1 | 39 |
| CA at 200 molar excess) | | |
| 1C5 CA at 10 molar excess | −22 ± 4 | 2 |
| 1C5 CA at 10 molar excess after masking | −3 ± 1 | 0 |
| removal | | |
| 1C5 CA at 30 molar excess | −28 ± 4 | 4 |
| 1C5 CA at 30 molar excess after masking | −4 ± 1 | N.T |
| removal | | |

*It is important to note that the number of masking agent units that were calculated from the total protein mass using MALDI TOF can be underestimated due to the acidic conditions used in this protocol measurement.

As with the masking of just the carrier, masking of the full conjugate can be removed in vitro by incubation at pH 4 with citrate buffer at 37° C. After 8 hours, full masking removal was achieved irrespective of the PEI level on the carrier (IEF results not shown).

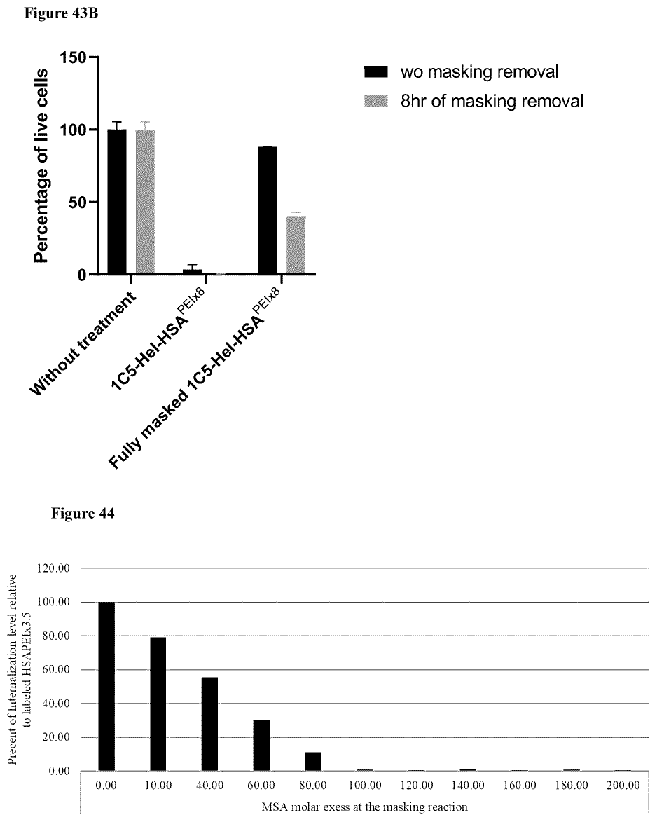

Activity of the payload that was masked was further tested after masking removal. For this purpose, binding ELISA to BRAF was performed for the anti BRAF payload (1C5) after masking removal. In this assay, the presence of carrier (HSA-PEI) was observed to give high unrelated signal, probably due to the stickiness of the highly positively charged protein to the ELISA plate. Therefore, in this case only, the payload, 1C5-Hel-LC, was used and was masked with citraconic anhydride (masking via positive lysine residues in the payload). The masked payload was tested for its ability to bind BRAF before and after masking removal, and the binding ability of anti-BRAF VHH was practically abolished following masking (FIG. 43A). However, masking removal fully restored binding. In addition, the change in the payload charge after masking and masking removal was evaluated by IEF (data not shown) and by zeta potential (Table 6) and one can clearly see the reduction in Zeta potential of the payload following its masking, which is correlated to the excess of the masking agent used. Additionally, it can be clearly seen that the acidic conditions have fully removed the masking as the Zeta potential of the payload was restored to its original value.

Similar to the conjugates in which only the carrier was masked (FIG. 30A-30B), the fully masked conjugates were also shown to regain their in vitro cytotoxic activity following masking removal. As can be seen in FIG. 43B, an 1C5 payload expressed with a rigid helical linker and further conjugated to a carrier with 3.5 PEI regained its activity after masking removal. Further, fully masked 1C5-HSA-PEI×3.5 and fully masked 1C5-HSA-PEI×8 had no effect on MEL-526 cells. These conjugates regained their activity following the in vitro masking removal, activity that was comparable to a conjugate of 1C5 that was not masked at all (FIG. 43B, data for PEI×3.5 not shown).

### Example 17: Partial Masking

In order to determine the level of masking that prevents internalization or, put another way, the level of positive charge that is needed for internalization, HSA-PEI×3.5 was labeled with an appropriate fluorescent dye (ATTO 542 or ATTO 647N) and the labeled protein was then masked with a non-reversible masking agent, methyl succinic anhydride (MSA), at different molar equivalents, to give carriers with a range of different levels of masking. The level of masking for each protein was evaluated by IEF and Zeta potential. The ability of the labeled carrier at different masking levels to internalize into cells was evaluated by incubation for 16 h with A375 cells followed by detection of cells with fluorescent signal via flow cytometry. Fluorescent labeling was performed on HSA^{PEI×3.5} using 2.5 molar excess of ATTO-542 with a Maleimide moiety (ATTO-TEC, Cat. No. AD 542) directed to the modification on the free cysteine at position 34 of the HSA or on HSA^{PEI×3.5} using 5 molar excess of ATTO-647N with a Maleimide moiety (ATTO-TEC, Cat. No. AD 647N-41). For this assay, non-reversible masking was used rather than reversible masking (with citraconic anhydride) to avoid the possibility of masking removal during the internalization assay. IEF analysis showed a strong correlation between pI and the level of MSA reagent used in the masking reaction (data not shown). The results of ATTO 542 are summarized in Table 7.

TABLE 7

| Construct | Zeta potential [Mv] |
|---|---|
| HSA-PEI × 3.5-ATTO 542 | 7 ± 3 |
| HSA-PEI × 3.5-ATTO 542 MSA 10 molar excess | 5 ± 2 |
| HSA-PEI × 3.5-ATTO 542 MSA 20 molar excess | 9 ± 4 |
| HSA-PEI × 3.5-ATTO 542 MSA 40 molar excess | 3 ± 2 |
| HSA-PEI × 3.5-ATTO 542 MSA 60 molar excess | −4 ± 0.3 |
| HSA-PEI × 3.5-ATTO 542 MSA 80 molar excess | −15.6 ± 1.5 |
| HSA-PEI × 3.5-ATTO 542 MSA 100 molar excess | −10.6 ± 2 |
| HSA-PEI × 3.5-ATTO 542 MSA 120 molar excess | −17.8 ± 4 |
| HSA-PEI × 3.5-ATTO 542 MSA 140 molar excess | −19.8 ± 2 |
| HSA-PEI × 3.5-ATTO 542 MSA 160 molar excess | −15 ± 5 |
| HSA-PEI × 3.5-ATTO 542 MSA 180 molar excess | −12 ± 4 |
| HSA-PEI × 3.5-ATTO 542 MSA 200 molar excess | −15.4 ± 3 |

Zeta potential measurements have been performed for ATTO 647N labeled constructs (unmasked and masked with various molar excess of MSA), resulting in similar results with up to 60 molar excess of MSA exhibited a positive zeta potential, and 80 molar excess of MSA and more (up to 200 molar equivalents) exhibited a negative zeta potential.

A375 cells (0.5×10\hat{ }6 per well), were seeded for 12 h then 20 (or 50 ng) of labeled carrier (HSA^{PEI×3.5} ATTO 647N) was added to the well. Samples were incubated for 16 h. At the end of the uptake period, the upper media was washed out, cells were detached from the plate and washed with cooled PBS. The amount of carrier internalized the cells was evaluated by ATTO 647N detection on flow cytometry, compared to isotope control sample. Internalization was reduced in proportion to the masking level increase (FIG. 44). Furthermore, internalization levels equivalent to at least 20% of the internalization achieved with HSA-PEI×3.5 without masking was achieved when 60 molar excess of MSA was used in the reaction or less. All these samples gave positive values in the zeta potential analysis, whereas the samples with negative values did not show internalization of even 10%. Thus, a zeta potential below zero is required for functional masking. Based on the fact that citraconic anhydride masking introduces a negative charge to the conjugate, it is presumed that about 50% or more of the amine groups (e.g., amine groups of PEI) needs to be masked. Thus, the inventors presumed that the molar ratio between the protected amines to unprotected amines in the protein conjugate of the invention (e.g., within the cell penetrating moiety) is at least about 7:10, at least about 1:1, or comprises an excess of the protected amines, such as between about 1:1 and 100:1, including any range between.

### Example 18: Tumor Targeting—Anti-PSMA

While the masking provides enrichment of the conjugates in acidic target areas additional targeting may be beneficial. To this end, the inventors have explored the use of an additional targeting moiety. This can be any protein domain, or antibody-like structure that is selected for its ability to bind an extracellular marker on the target cells. A second VHH was selected as the targeting moiety and was expressed in tandem with the payload VHH which targets an intracellular target. The masking enables the use of a targeting moiety as it eliminates strong electrostatic binding and enables the masked conjugate to roam the plasma and organs to find the targeting moiety's target.

A VHH against prostate specific membrane antigen (PSMA) known as JVZ-007 was selected as the targeting moiety. PSMA is known to be presented on prostate cancer cells and has been used extensively to target various imaging agents to prostate tumors. A tandem agent was generated comprising JVZ-007 and the anti-BRAF, 1C5, VHH. Two tandemly expressed agents were generated, one with the anti-PSMA VHH expressed at the N-terminus and one at the C-terminus creating αPSMA (JVZ-007)-Hel-αBRAF (1C5)-Hel-L-Cys and αBRAF (1C5)-Hel-αPSMA (JVZ-007)-Hel-L-Cys, respectively. Both end with a C-terminal cysteine to enable conjugation to the carrier.

The binding of the two constructs (before conjugation to the carrier) to PSMA positive cells (LNCaP clone FGC, Prostate Carcinoma, ATCC Number CRL-1740) was evaluated by FACS and was compared to the binding of these constructs to PSMA negative cells (PC-3, Prostate Adenocarcinoma, ATCC number CRL-1435). Binding of the active VHH, 1C5, alone to these cells was also evaluated.

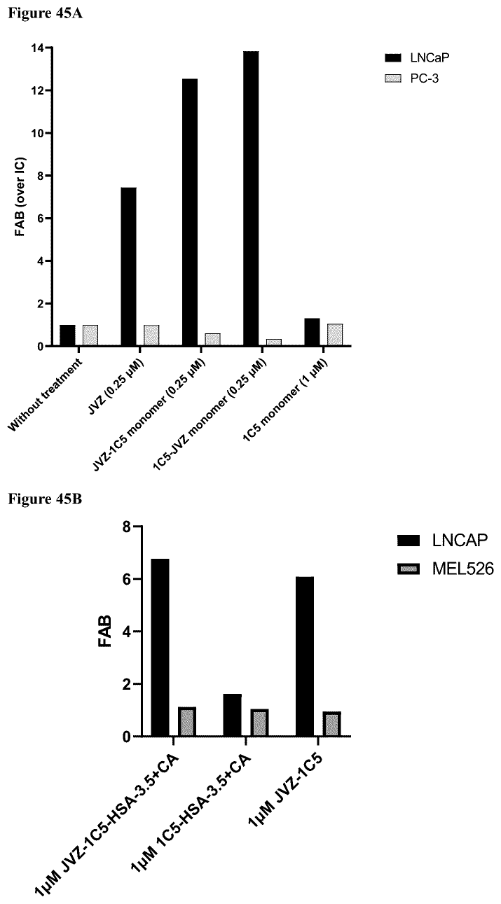

As expected, the anti-PSMA VHH strongly binds the PSMA positive cells and exhibits no binding of the PSMA negative cells. Interestingly, both the agents that contain the anti-PSMA VHH expressed in tandem with the anti-BRAF VHH also show very strong binding to the PSMA positive cells and no binding to the PSMA negative cells (FIG. 45A). Both constructs exhibit the same level of binding to the PSMA positive cells. Also as expected, the anti-BRAF 1C5 VHH, exhibits no binding to either the PSMA negative or positive cells. These results show that the tandem expression of a targeting moiety with another active moiety does not negatively affect the ability of the targeting moiety to recognize and bind its target.

The described agents were further conjugated to the masked carrier of the invention and their binding to PSMA positive cells was evaluated by FACS as before. The anti-PSMA containing conjugates (JVZ-1C5-HSA-PEI×3.5-CA) exhibited binding of the PSMA positive cells (LNCaP prostate cancer cells) and no binding of the negative cells (MEL-526 melanoma cancer cells) (FIG. 45B). This demonstrates that conjugation to the masked carrier does not inhibit binding to the target. The non-conjugated payload, JVZ-1C5, bound the PSMA-positive cells comparably to the conjugated payload indicating the carrier did not affect binding of the targeting moiety. The other anti-PSMA containing conjugate (1C5-JVZ-HSA-PEI×3.5-CA) also exhibited binding of the LNCaP cells and no binding of PSMA-negative cells (data not shown). None of the agents tested showed binding to PC3 prostate cancer cells which are PSMA-negative (data not shown).

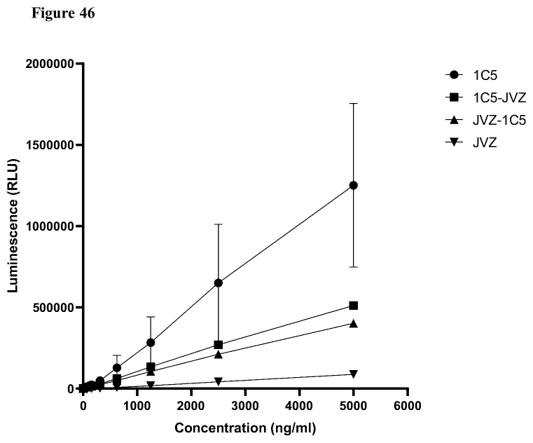

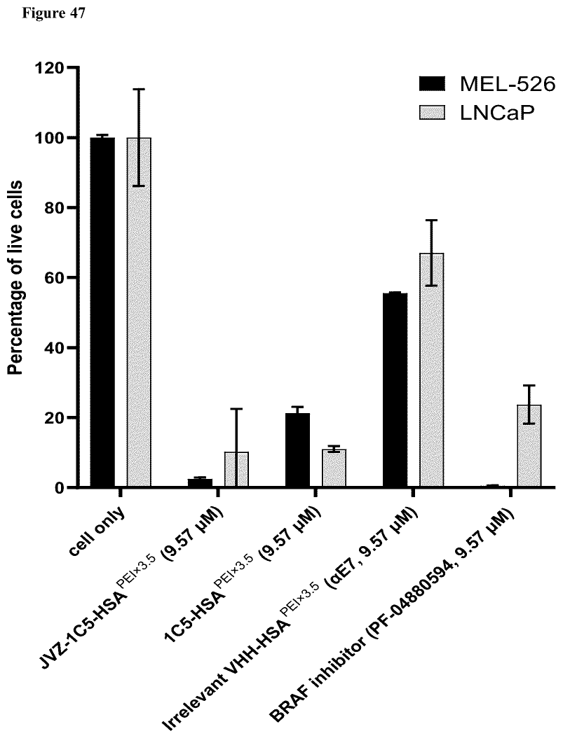

Having shown that expressing the anti-PSMA moiety in tandem with the anti-BRAF moiety does not interfere with its ability to bind PSMA, the ability of the tandem structures to still recognize and bind BRAF via the anti-BRAF moiety was tested. This was done via a recombinant BRAF binding ELISA. As can be seen in FIG. 46, both constructs expressing the anti-BRAF and anti-PSMA moieties in tandem retained their ability to bind BRAF, although the binding is somewhat weakened a bit as compared to the parent anti-BRAF VHH alone.

Next, the ability of the tandem construct with the unmasked carrier to inhibit intracellular BRAF and cause cell killing was evaluated. Cytotoxic activity of the anti-BRAF agent expressed with the targeting moiety was evaluated in two different cell lines, MEL-526 (PSMA negative) and LnCap (PSMA positive). In both cell lines the tandem agent exhibited cytotoxicity indicated that regardless of binding to the surface moiety the construct was internalized, escaped the endosomes and was able to modulate BRAF (FIG. 47). This cytotoxicity was of similar magnitude as the one exhibited by the anti-BRAF VHH alone conjugated to the same carrier. These results show the potential of these agents incorporating both a targeting moiety and an active moiety to serve as delivery agents.

The tandem expressed anti-PSMA-anti-BRAF (JVZ-IC5) is further conjugated to a masked carrier and it's in vivo biodistribution is evaluated. Athymic nude Foxn1nu mice are injected with PSMA-positive cancer cells (e.g., LNCaP tumor cells). Cells are injected subcutaneously at 10\hat{ }7 cells/100 μL, or at an appropriate amount to yield tumor initiation. Mice are injected with 1C5-masked HSA-PEI×3.5 or JVZ-1C5-masked HSA-PEI×3.5 at 250 nmol/Kg dose in 200 μL. Organs and tumors are collected at different time points from injection (e.g., after 2, 24, and 48 hours) and the amount of conjugate in the tumor and other organs is evaluated. ELISA (e.g., anti VHH ELISA) is used to evaluate total payload present. Imagining is also perform as described above and the distribution within the tumor and within tumor cell is evaluated. Total tumor weight in the two sets of treated mice is also monitored. Increased delivery to the tumor and into tumor cells is observed with the tandem JVZ-1C5-masked HSA-PEI×3.5 agent as the delivery moiety increases the targeting to the tumor. The increase in the tumor also leads to a concomitant decrease in other healthy tissues. Increase tumor cell killing, as measured by a decrease in tumor mass is also observed with the tandem molecule containing the targeting moiety.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A protein conjugate comprising a biological payload that interacts with an intracellular target, wherein the biological payload is covalently bound to a cell penetrating moiety comprising a plurality of amine groups, at least a portion of the amine groups is bound to a protecting group and said protecting group is capable of undergoing cleavage at a pH value of less than 7; and wherein said protein conjugate is characterized by a negative zeta potential.

2. The protein conjugate of claim 1, wherein the biological payload is linked by linker to a protein carrier covalently bound to said cell penetrating moiety.

3. The protein conjugate of claim 1, wherein said protein conjugate is characterized by an increased blood stability compared to an analogous protein conjugate devoid of the protecting group or an increased accumulation within a biological tissue having a pH value of less than 7, compared to an analogous protein conjugate devoid of the protecting group, optionally wherein said biological tissue is a tumor.

4. (canceled)

5. The protein conjugate of claim 1, wherein the plurality of amine groups comprises a primary amine, a secondary amine, or both; and at least 50% of the plurality of amine groups are bound to the protecting group.

6. The protein conjugate of claim 2, wherein
a. said linker is linked to said carrier, said payload or both by a covalent bond;
b. said protein carrier or biological payload comprises a plurality of PEI molecules;
c. said protein carrier or biological payload comprises between 2 and 30 PEI molecules;
d. wherein said protein carrier is human serum albumin (HSA);
e. wherein said protein carrier is HSA comprising between 3 and 10 PEI molecules;
f. said linker comprises a biocompatible polymer, a biodegradable polymer or both;
g. said linker comprises a biocompatible polymer comprising polyethlene glycol (PEG), optionally wherein said biodegradable polymer comprises a polyamino acid, wherein said linker further comprises a spacer covalently bound to (i) the biocompatible polymer or the biodegradable polymer and to (ii) the protein carrier, and wherein covalently bound is via a click reaction product;
h. said linker comprises a bio cleavable bond, optionally wherein said bio cleavable bond comprises a disulfide bond;
i. said linker is substantially stable in blood for at least 24 hours, and wherein said linker is a peptide linker;
j. said linker is demonstrates less than 25% cleavage in blood after 24 hours, and wherein said linker is a peptide linker;
k. said linker comprises a bio cleavable bond that is sterically hindered.

7. The protein conjugate of claim 1, wherein said protecting group comprises a moiety being negatively charged at a pH between 6 and 8.

8. The protein conjugate of claim 7, wherein at least one of:
a. said moiety comprises a carboxy group;
b. said protecting group is represented by Formula 1:
wherein n is an integer ranging from 0 to 5:represents an attachment point to the amine group, and represents a single bond or a double bond: R and R1 each independently represent a substituent selected from H, optionally substituted alkyl, optionally substituted cycloalkyl, optionally substituted aryl or heteroaryl, and carboxyalkyl, or any combination thereof; or R and R1 are bound together so as to form a cyclic ring:
c. said protecting group is represented by Formula 1:
wherein n is an integer ranging from 0 to 5; represents an attachment point to the amine group, and represents a single bond or a double bond; R and R1 each independently represent a substituent selected from H, optionally substituted alkyl, optionally substituted cycloalkyl and wherein one of R and R1 is H and another one of R and R1 comprises an alkyl or a carboxyalkyl; and
d. said protecting group is
including any salt thereof, wherein R and R1 are selected from H and methyl, and wherein R or R1 is methyl, optionally wherein said protecting group is derived from citraconic anhydride.

9. (canceled)

10. (canceled)

11. (canceled)

12. The protein conjugate of claim 1, wherein the cell penetrating moiety comprises a cationic polymer selected from a polyamine and polyethyleneimine (PEI), optionally wherein said PEI is a linear PEI or a branched PEI having a molecular weight of less than 2000 Daltons.

13. (canceled)

14. (canceled)

15. The protein conjugate of claim 1, wherein at least one of:
a. said biological payload is an antigen binding molecule that binds said intracellular target;
b. said biological payload is devoid of a disulfide bond that when cleaved diminishes interaction with said intracellular target:
c. said biological payload is selected from a single chain antibody, a single domain antibody, a variable heavy homodimer (VHH), a nanobody, an immunoglobulin novel antigen receptor (IgNAR), a designed ankyrin repeat protein (DARPin) and an antibody mimetic protein:
d. said protein carrier is devoid of DNA:
e. said biological payload does not bind a cell surface protein;
f. said protein conjugate further comprises a tag, optionally wherein said tag is conjugated to said biological payload:
g. said protein conjugate further comprises a targeting moiety that binds to a protein expressed on the surface of a target cell.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The protein conjugate of claim 2, wherein said protein carrier is HSA and said HSA comprises the amino acid sequence of SEQ ID NO: 1, or a fragment or homolog thereof comprising cysteine 34 (C34).

29. The protein conjugate of claim 28, wherein said linker is at least one of:
a. bound to said HSA via a disulfide bond;
b. bound to said C34 of HSA via a disulfide bond;
c. bound to said HSA via a disulfide bond proximal to said C34; and
d. bound to said HSA via a disulfide bond at a distance from said C34 from 5 to 15 angstroms.

30. (canceled)

31. (canceled)

32. (canceled)

33. (canceled)

34. (canceled)

35. The protein conjugate of claim 1, wherein said protein conjugate is characterized by a negative zeta potential of less than −1 mV.

36. (canceled)

37. (canceled)

38. The protein conjugate of claim 1, wherein said protecting group is derived from citraconic anhydride; optionally wherein the click reaction product is succinimide-thioether.

39. (canceled)

40. (canceled)

41. (canceled)

42. (canceled)

43. (canceled)

44. A method of producing a charge masked protein conjugate capable of binding an intracellular target, the method comprising:
a. providing;
i. a biological payload that binds said intracellular target, wherein the biological payload is covalently bound to a cell penetrating moiety comprising a plurality of amine groups; or
ii. a biological payload that binds said intracellular target and a protein carrier covalently bound to a cell penetrating moiety comprising a plurality of amine groups and providing the biological payload and the protein carrier under conditions sufficient for covalently binding said biological payload to the protein carrier via a linker to produce a protein conjugate covalently bound to a cell penetrating moiety comprising a plurality of amine groups; and
b. providing the biological payload under conditions sufficient for protecting at least a portion of the amine groups by a protecting group capable of undergoing cleavage at a pH value of less than 7, to obtain the charge masked protein conjugate comprising protected amine groups;
thereby producing a charge masked protein conjugate capable of binding an intracellular target.

45. (canceled)

46. The method of claim 44, further comprising at least one of:
a. determining stability of said linker in human blood, plasma or serum and in cytoplasmic conditions; and selecting a charge masked protein conjugate comprising a linker that is stable in said human blood, plasma or serum and unstable in said cytoplasmic conditions;
b. determining stability of said protected amine groups at neutral or basic pH and at acidic pH and selecting a charge masked protein conjugate comprising protected amin groups that are stable at neutral or basic pH and unstable at acidic pH, optionally wherein said determining is performed before formation of said protein conjugate or after formation of said charge masked protein conjugate:
c. contacting said charged masked protein conjugate with a cell and confirming said biological payload enters a cytoplasm of said cell:
d. selecting a targeting moiety that binds to a protein expressed on the surface of a target cell and conjugating said targeting moiety to said biological payload, said protein carrier or said protein conjugate, optionally wherein said targeting moiety is selecting a single chain antibody, a single domain antibody, a variable heavy homodimer (VHH), a nanobody, an immunoglobulin novel antigen receptor (IgNAR), a designed ankyrin repeat protein (DARPin) or an antibody mimetic protein;
e. selecting a targeting moiety that binds to a protein expressed on the surface of a target cell and conjugating said targeting moiety to said biological payload, said protein carrier or said protein conjugate and wherein said targeting moiety and said biological payload are comprised in a single polypeptide;
f. selecting a targeting moiety that binds to a protein expressed on the surface of a target cell and conjugating said targeting moiety to said biological payload, said protein carrier or said protein conjugate and wherein said targeting moiety and said biological payload are separated by a linker.

47. (canceled)

48. (canceled)

49. (canceled)

50. The method of claim 44, wherein said protein carrier comprises HSA.

51. The method of claim 44, wherein said cell penetrating moiety comprises at least one PEI.

52. The method of claim 44, wherein the charge masked protein conjugate is characterized by a negative zeta potential, is a protein conjugate of claim 1 or both.

53. The method of claim 44, wherein
a. the plurality of amine groups comprises a primary amine, a secondary amine, or both; and at least 80% of the plurality of amine groups are protected amine groups;
b. said protecting group comprises a moiety being negatively charged at a pH between 6 and 8;
c. said protecting group comprises a carboxy group;
d. said protein carrier or biological payload is covalently bound to at least 2 molecules of PEI, optionally wherein said protein carrier is covalently bound to at least 8 molecules of PEI;
e. said biological payload is devoid of a disulfide bond that when cleaved diminishes binding to said intracellular target;
f. said linker comprises a biocompatible polymer;
g. said covalently linking is via a click reaction;
h. the biological payload is covalently bound to a linker comprising a first reactive group; and wherein said protein carrier is covalently bound to a linker comprising a second reactive group having reactivity to said first reactive group; and wherein said conditions sufficient for covalently binding said biological payload to the protein carrier comprises reacting said first reactive group with said second reactive group, thereby covalently linking said biological agent and said protein carrier; and
i. said linker comprises a bio cleavable bond.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A protein conjugate produced by a method of claim 44.

72. A pharmaceutical composition, comprising the protein conjugate of claim 1 and a pharmaceutically acceptable carrier, excipient or adjuvant.

73. (canceled)

74. (canceled)

75. A method of binding an intracellular target, the method comprising contacting a cell expressing said intracellular target with the pharmaceutical composition of claim 72, wherein said biological payload binds said intracellular target, thereby binding said intracellular target.

76. The method of claim 75, wherein at least one of:
a. said method is a method of detecting an intracellular target and said protein conjugate comprises a detectable tag, and wherein said method further comprises detecting said detectable tag;
b. said method is a method of modulating said intracellular target and wherein said biological payload is an agonist or antagonist of said intracellular target;
c. said cell is in a subject and wherein said contacting comprises administering said pharmaceutical composition claim 72 to said subject;
d. said cell expresses a target surface protein and said protein conjugate comprises a targeting moiety that binds to said target surface protein;
e. said cell is in a subject and said method is a method of treating a condition in a subject in need thereof, wherein said condition is treatable by modulation of said intracellular target;
f. said method is for delivering biological payload to a specific tissue within a subject, wherein the specific tissue is characterized by a pH value of below 7; and
g. said method is for delivering biological payload to a tumour within a subject.

77. (canceled)

78. (canceled)

79. (canceled)

80. (canceled)

81. (canceled)

82. (canceled)

83. (canceled)

84. (canceled)
